SEQUENCE LISTING (42 sequences)

SEQ ID NO: 1 - protein, 422 aa, synthetic construct
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 2 - protein, 265 aa, synthetic construct
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 3 - protein, 2109 aa, synthetic construct
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 4 - RNA, 1269 nt, synthetic construct
  source 1..1269: mol_type = other RNA
atgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatga

SEQ ID NO: 5 - RNA, 798 nt, synthetic construct
  source 1..798: mol_type = other RNA
atggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtag

SEQ ID NO: 6 - RNA, 6330 nt, synthetic construct
  source 1..6330: mol_type = other RNA
atggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaa

SEQ ID NO: 7 - protein, 422 aa, synthetic construct
MSVTVKRIIDNTVIVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 8 - protein, 265 aa, synthetic construct
MDNLTKVREYLKSYSRLDQAVGEIDEIEAQRAEKSNYELFQEDGVEEHTKPSYFQAADDSDTESEPEIEDNQGLYAQDPEAEQVEGFIQGPLDDYADEEVDVVFTSDWKPPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 9 - protein, 2109 aa, synthetic construct
MEVHDFETDEFNDFNEDDYATREFLNPDERMTYLNHADYNLNSPLISDDIDNLIRKFNSLPIPSMWDSKNWDGVLEMLTSCQANPISTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWTILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRTSDSLAPIGNWIRSLELVRNQVRLNPFNEILFNQLCRTVDNHLKWSNLRRNTGMIEWINRRISKEDRSILMLKSDLHEENSWRD

SEQ ID NO: 10 - RNA, 1269 nt, synthetic construct
  source 1..1269: mol_type = other RNA
atgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatga

SEQ ID NO: 11 - RNA, 798 nt, synthetic construct
  source 1..798: mol_type = other RNA
atggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtag

SEQ ID NO: 12 - RNA, 6330 nt, synthetic construct
  source 1..6330: mol_type = other RNA
atggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaa

SEQ ID NO: 13 - protein, 422 aa, synthetic construct
MAPTVKRIINDSIIQPKLPANEDPVEYPADYFKNNTNIVLYVSTKVALNDLRAYVYQGIKSGNPSILHINAYLYAALKGVEGTLDRDWVSFGRTIGKREENVKIFDLVKVEELKTALPDGKSDPDRSAEDDKWLPIYILGLYRVGRSKVTDYRKKLLDGLENQCRVASTRFESLVEDGLDFFDIWENDPNFTKIVAAVDMFFHMFKKHERAPIRYGTIVSRFKDCAALATFGHLSKVSGLSIEDLTTWVLNREVADELCQMMYPGQEIDKADSYMPYMIDFGLSQKSPYSSVKNPAFHFWGQLAALLLRSTRAKNARQPDDIEYTSLTCASLLLSFAVGSSADIEQQFYIGEDKYTTEKDDSLKKSDVPPKGRNVVDWLGWYDDNGGKPTPDMLNFARRAVSSLQSLREKTIGKYAKVEFDK

SEQ ID NO: 14 - protein, 274 aa, synthetic construct
MDSVDRLKTYLATYDNLDSALQDANESEERREDKYLQDLFIEDQGDKPTPSYYQEEESSDSDTDYNAEHLTMLSPDERIDKWEEDLPELEKIDDDIPVTFSDWTQPVMKENGGEKSLSLFPPVGLTKIQTEQWKKTIEAVCESSKYWNLSECQILNLEDSLTLKGRLMTPDCSSSVKSQNSVRRSEPLYSSHSPGPPLKVSESINLWDLKSTEVQLISKRAGVKDMTVKLTDFFGSEEEYYSVCPEGAPDLMGAIIMGLKYKKLFNQARMKYRL

SEQ ID NO: 15 - protein, 2109 aa, synthetic construct
MDFDLIEDSANWEDDESDFFLRDILSQEDQMSYLNTADYNLNSPLISDDMVYIIKRMNHEEVPPIWRSKEWDSPLDMLRGCQAQPMSHQEMHNWFGTWIQNVQHDSAQPFTFLKEVDKESEMTYDLVSTFLKGWVGKDYPFKSKNKEIDSMALVGPLCQKFLDLHKITLILNAVSLGETKELLTTFKGKYRMSCENIPIARLRLPSLGPVFMCKGWTYIHKERVLMDRTSFSCVKDVIIGRMQTFLSMIGRSDNKFSPDQIYTLANVYRIGDKILEQCGNKAYDLIKMIEPICNLKMMELARLHRPKIPKFPHFEEHVKGSVQELTQRSNRIQTLYDLIMSMKDVDLVLVVYGSFRHWGHPFIDYFQGLEKLHTQVNMEKHIDKEYPQQLASDLARLVLNKQFSESKKWFVDPSKMSPKHPFYEHVINKTWPTAAKIQDFGDNWHKLPLIQCFEIPDLIDPSVIYSDKSHSMNKKEVIQHVRSKPNIPIPSNKVLQTMLTNRATNWKAFLKDIDENGLDDDDLIIGLKGKERELKIAGRFFSLMSWRLREYFVITEYLIKTYYVPLFKGLTMADDLTSVIKKMMDSSSGQGLDDYSSVCLANHIDYEKWNNHQRKESNGPIFRVMGQFLGYPSLIERTHEFFEKSLIYYNGRPDLMTIRNGTLCNSTKHRVCWNGQKGGLEGLRQKGWSIVNLLVIQREAKIRNTAVKVLAQGDNQVICTQYKTKKTRSELELRAVLHQMAGNNNKIMEEIKRGTEKLGLIINDDETMQSADYLNYGKIPIFRGVIRGLETKRWSRVTCVTNDQIPTCANLMSSVSTNALTVAHFAENPINAMIQYNYFGTFARLLLFMHDPAIRQSLYKVQEKIPGLHTRTFKYAMLYLDPSIGGVCGMALSRFLIRAFPDPVTESLSFWKFIYEHASEPHLKKMEVMFETPQGAKFRIEHINKLLEDPTSLNISMGMSPANLLKSEVKKCLIESRSSIKNEIIKDATIYMHQEEEKLRGFLWSIKPLFPRFLSEFKAGTFLGVSEGFINLFQNSRTIRNSFKKRYHKDLDELIIKREISSLSHLGSMHYRLGDNQIWSCSASRADILRYKSWTRKVVGTTVPHPLEMHGPPSKKERPCQLCNSSGLTYISVHCPKGIIDVFNRRGPLPAYLGSNTSESTSILQPWEKESKIPIIKRATRLRDAISWFIPPESPLSTCILNNIQALTGEDWSSKQHGFKRTGSALHRFSTSRMSNGGFASQSPATLTRMIATTDTMRDFGTKNYDFMFQASLLYGQMTTSISRYGTPGSCTDHYHIRCKGCIREIEEVELNTSLEYKTPDVSHILEKWRNNTGSWGHQIKQLKPAEGNWESLSPVEQSYQVARCIGFLYGELTHKKSRQADDSSLFPLSIQLKVRGRGFLRGLLDGLMRSSCCQVIHRRSVSTLKRPANAVYGGLIYLIDKLSASSPFLSLVRTGPIRQELEQVPHKMSTSYPTNIRDLGSIVRNYFKYQCRPVERGNYKTYYNQIWLFSDVLSTEFIGPMRISSSLLRLLYRPSLTKKDREELRELAALSSNLRSGEDWDDLHIKFFSNDLLFCSQEIRHACKFGIKKDNEDITFYPNWGTEYIGNVTDIPVFYRAQNVKKDIRVPPRIQNPLMSGLRLGQLPTGAHYKMRAIVFRLKIPYHDFLDCGDGSGGMTAALLRYNRTSRGIFNSLLDLSDTMLRGSSPEPPSALETLGGERVRCVNGDSCWEHPSDLSDENTWKYFLHLKKGCGMSINLITMDMEVQDSVISYKIESLVRQYVPVLLESDGCLIYKTYGTYIATQEDNSLTLIGSLFHSVQLVQTDLSSSNTSELYLVCRRLKDYVDTPFVDWIELYDNWEKQYAFRSFKDEFQRAQSLTPETTLIGIPPQFVPDPGVNLETLFQIAGVPTGVAHGITHHILQSKDKLISNAIGSMCVISHFHINTIRTTDSMPGPPSDGDVNKMCSALIGACFWLDWMESDLNLYKHCLRSIMKSMPVRWFRTLKNEKWSQKWDCKGDAIPKDSRLGDSLANIGNWIRAWELIRNGNKSEPFDSMVAEALTKSVDKSLSWRKISKSTGIPRLLNSDIDLVDQSILNVQIDIVENQAWQN

SEQ ID NO: 16 - RNA, 1269 nt, synthetic construct
  source 1..1269: mol_type = other RNA
atggctcctacagttaagagaatcattaatgactccataattcagcctaaactaccggccaatgaggatccggtagagtatccagctgattacttcaaaaataacaccaacatagtattatatgtgagcactaaagtagccctaaatgatttaagggcctatgtctaccagggaatcaaatccggtaatccatcaatactccacataaatgcttatctttatgccgcgttaaagggagtagaaggaacattagacagagactgggttagctttggaagaacgatcggaaagagggaagagaatgtcaaaatcttcgatctagtcaaagttgaggaactgaagacagctcttcctgatgggaaatcggatccagatcgatcagctgaagatgataaatggcttcctatttacatcctaggcctatatcgagtgggtagatccaaagttacagattacagaaagaaactcctggacggtctagaaaatcagtgtagagtggcatcaaccagatttgaaagccttgttgaagatggtcttgacttctttgacatctgggaaaatgatcccaacttcaccaaaatagttgctgcagtggatatgttttttcacatgttcaagaagcatgaacgtgctccaatcagatatggaaccatagtctcacgattcaaagactgtgcagcacttgctacatttggacatctcagcaaggttagcggactttcaattgaagacctaacaacatgggttctgaatcgagaagtcgcagacgaactatgccaaatgatgtacccaggccaagaaattgacaaggcagattcatacatgccatatatgattgatttcgggttatcccaaaaatctccttattcatcagtgaagaatccagctttccacttctggggtcaactggccgcactcttgctaagatcaactcgagcaaaaaatgctcggcaacccgatgacattgagtacacttcgctgacttgtgcaagtttactactgtcatttgcagttggatcttcagcagacattgaacaacaattctacatcggagaggacaaatacacaacagagaaagatgacagtctcaagaaatctgatgtcccaccgaaaggaagaaatgttgtagactggctcggttggtatgatgacaatggagggaaacccacaccggatatgctcaacttcgcacgaagagcagtcagttcgttgcagtcactccgcgagaaaacaattggcaaatacgcaaaagtagaatttgacaaatga

SEQ ID NO: 17 - RNA, 825 nt, synthetic construct
  source 1..825: mol_type = other RNA
atggacagtgttgataggctcaagacttacttagccacttatgataatttggattctgccttgcaggatgccaatgaatctgaggaaagacgagaggataaatatctccaagacctcttcatcgaagatcaaggagataaaccaactccgtcatattatcaggaagaagaatcgtcagattcagatactgattataatgctgaacatcttacgatgctgtcaccggatgaaagaatagacaagtgggaagaagatttgcctgaattagaaaagattgatgatgatataccggtgaccttttctgattggacacagcctgtaatgaaggaaaatgggggagagaaatcattgtctctgttccctccagtcgggttaacaaagattcaaacagaacaatggaaaaaaaccattgaggcggtttgtgagagttcaaaatattggaatttatcagaatgccaaattcttaacttggaagacagcctcactctcaaaggccgattgatgactcctgattgtagttcttcagtaaaatctcaaaattctgtccggaggtcagaacctctctactcctctcattctccaggtcccccactcaaggtatcagagtccatcaatttatgggatttaaagtccactgaagtacaattgatctccaagagagccggagttaaggacatgacagtcaaattgacagacttctttggaagtgaggaagagtattattcagtatgcccagaaggggcgccagacttgatgggagctatcatcatgggactgaagtacaagaaactcttcaatcaggcaagaatgaaatatcgtctttaa

SEQ ID NO: 18 - RNA, 6330 nt, synthetic construct
  source 1..6330: mol_type = other RNA
atggatttcgatctaatagaagattctgctaactgggaagatgatgagtcggacttttttttgcgagacattctatctcaagaagatcagatgtcttacttaaacaccgcagactataatttgaattctccactcatttccgatgacatggtttatataattaaaagaatgaatcacgaggaggttccccccatttggagatctaaggagtgggattctcctcttgatatgcttagaggttgtcaagctcaacccatgtctcaccaagagatgcacaattggtttggaacttggattcaaaatgttcagcatgattccgcccaacccttcacattcttaaaagaggtggacaaagagtcagaaatgacctatgacttggtctccacttttctgaaggggtgggtggggaaagattacccattcaagtcaaagaacaaagaaattgatagtatggcattagtcggtcctctttgtcaaaaattccttgatttgcataaaatcactctgatattaaatgcagtttctttgggggaaactaaagaactcctaaccactttcaagggtaaataccgcatgagttgtgaaaacattcctattgctcgcttaagacttccaagtctaggccctgtattcatgtgcaaaggttggacttatatccacaaagaaagagtgctaatggatcgaacttccttctcatgtgtaaaagatgtgataataggacgaatgcagacatttttgtctatgatcggacgatcggataacaagtttagtcctgatcaaatctatactctggcaaatgtatacagaattggagataaaattttagaacaatgcggcaataaggcttatgatttgattaaaatgattgaacccatctgcaatctgaaaatgatggaattagctagattgcatcgtcccaagattccgaagttccctcattttgaagagcatgtcaaaggctcagtgcaagaattgacacaaaggtccaatagaatacaaacattgtatgatctgattatgtctatgaaggatgttgacctcgtattagtggtttacggatctttccgtcattggggtcacccattcatcgattattttcagggattggaaaaattgcacactcaagtgaatatggaaaagcatattgacaaggagtacccacagcaattggccagtgacttagccagactagtattaaataaacaattcagcgaatcaaaaaaatggtttgtagacccttcaaaaatgtctcccaaacaccctttttatgaacatgttatcaataagacatggcctacagcagctaaaattcaagactttggagacaattggcacaagctcccattgattcaatgttttgaaataccagacttgatagatccatcagtgatttattcagacaaaagtcattccatgaacaaaaaggaggtgatacagcatgttcggtctaagccaaatattcccatcccaagcaacaaagttctgcaaacaatgcttaccaatagagcaacaaattggaaagcctttctgaaagatattgacgaaaatggactggatgatgacgatttaataatcggattaaagggaaaagaaagagaattgaaaatagctgggagattcttctcattaatgtcatggagattaagggaatattttgtgatcacagagtacctaatcaagacatactacgttcccttgtttaaggggttaactatggcagatgacctcacatcggtgatcaagaagatgatggacagttcatcaggtcaagggcttgatgattattcatctgtttgtttagccaaccatatcgattatgagaagtggaataaccatcagagaaaggaatcgaatggtcccatcttcagagtcatgggacagtttctaggttatccatcacttatagagagaactcatgagttttttgagaagagcttaatttactataacgggcgtccagatttgatgacaattcgaaatggtacattgtgtaattccacgaagcaccgagtttgctggaatgggcagaaaggaggattggaaggactaagacaaaagggatggagtattgtgaatctcttggttattcaaagggaagctaagatccgaaacacagcagtgaaggtcttagcacagggagataatcaggttatatgcactcaatacaagacaaagaagactagatcagagttagagttgagagcagtcttacatcagatggccgggaataataacaaaattatggaagaaattaagagaggtacagaaaagttgggcctaatcataaatgatgatgagactatgcaatcagcagactatttgaactatgggaaaattccaattttccgaggagtcatcagaggactagaaacaaagagatggtcacgggtgacatgcgtcacaaatgatcaaatcccgacgtgtgccaatttgatgtcctcagtctctactaatgccttgactgttgctcattttgctgaaaatcctataaatgcaatgattcaatacaattactttggaacctttgcacgattattgctttttatgcatgaccccgcaatccggcaatccttgtataaggtccaagaaaaaatacctggtttgcacaccagaacattcaaatacgccatgttgtacctagatccttcaatcggaggagtatgtggtatggcattatctcgttttttaattagagcatttccagatccagtaacagagagtctctcattctggaagtttatctatgaacatgcttctgagcctcatctaaaaaagatggaagtgatgttcgagaccccccaaggtgccaaattcagaatagaacacatcaacaaattgttagaggaccctacctctctaaacatctcaatgggtatgagtcccgcaaatttgttaaagagtgaggtcaaaaaatgtttaatagaatcaagatcatccatcaagaatgaaatcataaaagatgctactatctacatgcatcaagaagaagagaagcttagaggatttttgtggtccatcaaaccattattccctcgtttcttgagtgaattcaaagctggaacttttttgggagtatctgagggctttataaatttatttcaaaactcacgtaccataagaaactcatttaaaaagagatatcacaaggatcttgatgaattaataatcaagagagaaatatcgtccttgagtcatctcggatctatgcattatcgattaggggataatcagatatggtcctgttctgcatctcgagccgacatattaagatacaagtcatggaccaggaaagttgtgggaactacagtgcctcatcctttggaaatgcacggaccaccctcaaagaaggaaagaccttgccaattgtgtaattcatccggtctcacatatatttcggtacattgtccaaaaggaattattgatgtattcaatagaaggggacctttgccggcttacttggggtccaatacatctgagtctacatctattttacagccgtgggaaaaagagagtaaaataccaataattaaaagagcaactagattaagggacgccatttcttggttcatcccacccgagtctcctctgtctacatgcattttgaataatattcaggctttaacaggagaagattggagctctaaacaacatggctttaagaggacaggatcagcattacatagattttccacctcccggatgagcaatggaggatttgcctcacaaagcccggccaccttaactcgcatgattgcaactacagacacaatgagagactttggtacaaaaaattatgatttcatgttccaagcatctttgttatatggacagatgacaacaagtatttcaagatatggaaccccagggtcttgcacggatcattaccatatcagatgtaaaggatgcattagagagattgaagaagtagaactgaacactagtctagaatacaagacgcccgatgtttctcacatattggaaaaatggaggaacaatactggatcttggggtcatcaaatcaaacaattgaaacctgccgagggaaactgggaatcattgtctcctgtagagcaatcatatcaagttgcaagatgtattggatttctttatggtgaactgacacacaagaaatcgagacaggctgatgatagttctctattccccttgagcatccaactaaaagtgagagggagaggtttcttgcgaggccttctagacggtttgatgagatccagttgctgtcaggtaattcatagacgaagtgtttctaccttaaagagacctgcaaatgcagtttacggtggcctcatatacctcattgataaactaagtgcctcaagtcccttcttatcacttgtaagaaccggacctattcgacaagaattagaacaggtgccacacaagatgtctacatcgtatccaacgaacattagggacttgggctccattgtgagaaattattttaaatatcagtgtcgaccagttgagagaggaaactataaaacttattataatcaaatatggttattttctgatgttttgtccactgaatttatagggccaatgcgtatatccagctctctccttagactcctttatcgaccttctctgacaaagaaggatagagaagaattgagggagttagcagcactatcatctaatttacgaagtggagaagattgggatgatttacatatcaaatttttctcaaacgatctcctcttttgctcacaagagataagacatgcttgcaaatttgggattaaaaaagataatgaagatatcactttttacccgaattggggaacagagtacattgggaatgtcactgatattcctgtgttttaccgagctcaaaatgtcaaaaaggatattagagtaccccctcggatccaaaatcctttaatgtcaggactcaggcttggacaactgccaactggggctcattacaaaatgagagccattgtatttcgtcttaaaattccgtatcatgactttctggattgcggagatgggtcaggaggaatgactgcagcattactccggtataataggacatccagaggaatctttaacagtttgttggatctgtcagacacaatgctaagaggatcctcaccagaacctcccagtgcccttgagaccctggggggagagagagtaagatgtgtgaatggagacagttgctgggaacatccttccgatctcagcgatgagaacacatggaagtattttctccatttaaaaaagggatgtggaatgagtatcaatcttattaccatggatatggaagtccaggattctgttatatcatacaaaatcgagtcattggtcagacagtatgttccggttctattggaatccgacggttgccttatttataaaacatatggaacatatattgccacacaagaagacaattccctgactcttataggatcactttttcactctgtccaacttgtccaaacggatttaagctctagtaatacatccgaattgtacttggtatgcaggagattgaaggattatgtagacactccctttgttgattggatagaattatacgacaactgggagaaacagtatgctttcagaagcttcaaagatgaatttcaaagagctcaatcacttaccccagagacaaccctaataggaatacctcctcaatttgtaccggatcctggagtcaatctagagaccctgtttcaaatagcaggggttcccaccggagttgcgcatggaatcacacatcacatattgcagtctaaggataaattaatatcaaatgccatcggaagcatgtgtgttatctctcatttccacattaacacaatacggaccacagatagtatgcctggacccccatcagatggggatgtcaacaaaatgtgttcagcattgattggggcatgtttctggctggactggatggaatctgatcttaacttatacaaacattgtctaagatcaataatgaagtctatgcctgtgagatggtttagaacattaaaaaatgaaaaatggtcgcaaaaatgggattgtaaaggagatgcaatcccaaaagattccagattaggagacagccttgctaatatcggcaattggataagagcttgggaattaattagaaatggaaataagtctgagccttttgattcaatggtagcagaagcattgacaaaatctgtggataaatcacttagttggaggaaaatctcaaagtcaactggaattccgagacttctaaacagtgatatcgatttggttgatcaatctatactaaatgttcagatcgacatcgtagaaaatcaagcttggcaaaattga

SEQ ID NO: 19 - protein, 422 aa, synthetic construct
MSSQVFCISTGQTVSVCLPANEDPVEFPGAFFTPNARKPTVYIKKETDLSLLRSHVYDGIKDGSVTVSQINSYLYMVLKDIREKPDKNWTSFGVELGKKNEPMGIFDLLNVEDVKGKELDKKGQDTRLPGDDLWLPTLILGLYRVSRATQVEYKKTLMTNLYAQCKLRTKDAEEIVDETAEFFNAWANDSNFTKIVAAVDMYFHHFKKSDHAPIRFGTIVSRFKDCAALSTLSHLQKVTGLPIEEVFTWVFNKSVQDDLLRMMTPGQEIDQADSYMPYLIDMGLSTKSPYSSTKNPSFHFWGQLTAFLVKSARAKNALVPVDIAYHELTTAALLFAYAIGRSSELEQRFVLNGKKFTKEKDSRDDNDTTPPSERNVVVWLAWWEDIKHEITPDMKAFAKRAVERVGDIRVNSVAEYARKLFA

SEQ ID NO: 20 - protein, 293 aa, synthetic construct
MEDSQLYQALKNYPKLQDTLDSIENLEDDTKSEPSECGSPTERGIPSYYLAEELDECEEEDSEDDDDNLPTEIPDPPTVDMLEAIMEDEIDDTAYQVHFEAKQTWKPVIETGGNERGKFTLSVPQNLSALQLLQWETGIHALAERLGGCRLLQISTRGTRDGIEFTVRETPCVSPASDPIPSTSRSSSIASNVSTRQTESPGSKSNTSLGIPEVPANLIDIGAIDKEFILAAISPSDPPYKNTLRNLFGSGDSFEQYNQTGIYSLKELVVAGLKRKGIYNRIRIRCHLEPQFN

SEQ ID NO: 21 - protein, 2092 aa, synthetic construct
MDLNPVDDAAELSEENFFSGKLSKECRIRGLNSVDYNLNSPLVSDDLTYLLNKFKGKPVPIRWKMKKWDSILDQLRKHDLEYLRPSDLHQWFAEWMLYSKHGSKQGEDFLKTVDEEASDTFEVVRSFIRGWTGGEINFVRKSGKHMGYCAELCQKFLDLHKLTLLGNAATDNELLQLSKTFGDDKIYKKRLIKLPSLGRVIFDSGFFIVLDQRVLMDRNFMLMMKDVIIGRMQTVLSMISRCDDKFSSKDIDFLLKVYSTGDKIIRKLGNDGYELIKTVEPMCNLRLSDLARRFRPLVPPFPHFRRHIESTVDELSAKTPLIRELFSLIDTSPNVDSTLVVYGSFRHWGHPFINYFEGLEKLHKQVTMEKEIDTNYSEALASDLARIVLTKEFNEKKQWAVDYHRVPTSHPFKNHIRDNTWPTAAVIQDFGDHWHELPLIQCFDIPDLIDPSIIYSDKSHSMNRSEVLNHVRTKPHTPIPSKKVLESMIDKPATNWLEFLEEIDKNGLSDEDLVIGLKGKERELKIAGRFFSLMSWKLREYFVVTEYLIKTHFVPLFHGLTMADDMTAVIKKMLESSSGQGLTNYDSVCIANHIDYEKWNNHQRKLSNGPVFKVMGQFLGYPNLIYRTHEFFEKSLIYYNERPDLMKVRNGILENSTHQRVCWNGQAGGLEGLRQKGWSILNLLVIQREAKIRNTAVKVLAQGDNQVICTQYKTKQYRNDIELRQALNQMAANNDVIMKAIESGTNKLGLLINQDETMQSADYLNYGKVPIFRGVIRGLETKRWSRVTCVTNDQLPTCANLMSSVSTNALTVAHFDVHPLNAMIQFNFFGNFARLLLIMHDPAIRQSLNQLKGPNINVHSYGFKVAMLYLDPSIGGVCGTALSRFLIRSFPDPVTESLSFWKLIHHSTSDIRLKNLSEQFGNPKIAVFRESHIEKLLEDPTSLNISMGMSAANLLKTEIKKNLLQKKSSIGNQIVKDAVYYIHSEDEKLRTFLWSITPLFPRFLSEFKAGTFMGVASSIVSLFQNSRTIRNVFRDYMSQTIDDLIVKSELTSLEHLSNYTDRKGSGGIWSCSAEQADKLRRMSWKRPVLGTTVPHPLEMHGRGTLKSPLSKCCKESRMDYISVHIPEGLNKVLDGRGSLPAYLGSKTSESTSILQPWEKESKIPIIRRATRLRDAIHWFVDPDSNLARSILNNIESLTGEKWEGALKGYKRTGSALHRFSTSRVSHGGFSSQSPACLTRMMATTDTMRDYAQLNYDFMFQASLLYSQMTSSVILMGTTVSNTIHFHVTCRKCIREITEPMLESPREYRGKDVHLVLAKWKNSSNGWGETLQLLKPVEGDWDTIPPVEKSYHVGRILGFLYGDLKSQNSSRADDSSIFPLSIQMRLRGRGFLRGILDGLVRASACQVIHRRSVALLSKPANAIYGGLIYLIDKISASTSFTTLCRDGPIREELSSIPHKIPTSYPTSNSDMGLHIRNYLKFQCKTVELGKYQSDIKDLWLFSDVMTSNIAGPFALSTKILKCLYKPALSQKDRNNIRKISNFSKMMRSQLSWDPTSSEFITSQILVCNEEIRHACKFGIPKLSLKFDDPVWGPEDYGLIWSIPVDYSSQSVPKNLKPCPRIQNPSISGFRLGQLPTGAHYKLRSILRKKNIHYRDALCGGDGSGGMTAAVLRYNLKARAIFNSILDFDGSTMKGASPDPPSALETVVNGRTRCVNAESCWENPSDLSEQRTWDYFKFLKTHHGLKIDLIVLDMEVRDFAISASIEKCVRNNVSSILEEDGVLIYKTYGSTIAAESSNAVVNIGVLFESVELIQTEYSSTSTSEVYMYCRKIKKFVDAPHPDWISLDYYWSKLFCFRSYKEEFFRSYEVSRKESLKGIPNSFIPDPLVNLETLLQIAGVPSGISHQLAIDIKESQLTQITAAMVLCGMIANYTLDVTKKRDSYNPPSDGRLIRMSAALVGISFWISVKYYDKELNFELEQILSRSFPIRWMLSRNYLFWTTKKGFRNAKDVRLSGNMANIGNWIRCMELLHLPPGSLSKDEVTTTCGKYIRSLKYSVILQQTGIIDLWKSRVASDDRSLMEVKTEFIESEHWVD

SEQ ID NO: 22 - RNA, 1269 nt, synthetic construct
  source 1..1269: mol_type = other RNA
atgagttctcaagtattctgcatttctacaggtcaaacagtctccgtatgccttccagcaaatgaagacccagtagagtttccaggggcgttcttcactccaaatgctaggaaaccgacggtgtacataaagaaggagacagatctgagtcttttgaggagtcatgtttatgacgggattaaggatggttctgtgacagtgtcccagataaattcttatctatacatggttttgaaagatatccgggagaaaccagataaaaattggacttctttcggagtcgaattgggaaaaaagaatgagccaatgggaattttcgatctgttgaatgttgaagatgttaaggggaaagaattggacaaaaaagggcaagacacccgactgcccggagatgatctatggcttccaacgctgatcctcggtctttatcgcgtgtccagggcgacacaggtcgaatacaagaagactctgatgaccaatctatatgctcagtgtaaattgcgcacgaaggatgcagaggagattgtggatgaaacagcagaattcttcaatgcttgggcaaatgactccaacttcacaaaaatcgtagctgctgtggatatgtacttccaccacttcaagaagagtgatcatgctcctattcgtttcggaacaattgtgtctagattcaaagactgtgcagcattgtccactctgtcgcacctccaaaaggtcacagggctccccattgaggaggtcttcacctgggttttcaataaatcagtacaggatgatttgttgcggatgatgactcctggccaggaaattgaccaagctgattcctatatgccctatctgatcgacatgggcttgtccacgaagtctccttactcttcaacaaaaaatcccagtttccatttctgggggcaactgaccgcattcttggtaaaatccgcaagagcaaagaatgcgttagtcccagttgacatcgcataccatgaactgaccactgctgcactgctttttgcctatgcaattggcagatcgtctgagctggagcagagattcgtgcttaacgggaagaagttcaccaaggagaaggactctcgagatgacaatgacactactccgccatcggagagaaatgttgttgtttggctggcctggtgggaggacattaagcatgaaattactccggacatgaaggcctttgccaagagagcagtggaaagggtaggagatattcgagtgaactcagttgcagagtatgccaggaaactctttgcatga

SEQ ID NO: 23 - RNA, 882 nt, synthetic construct
  source 1..882: mol_type = other RNA
atggaagactctcaactatatcaagctctcaagaattacccaaagcttcaggacactctcgactcaattgagaatcttgaagatgacacaaagtcagagccatctgagtgtggttcccccacagagagaggaattcccagctattacctggctgaagaattggatgaatgtgaggaagaagattcggaagatgatgatgacaatttacctactgagataccggatcctcctactgttgacatgctggaggcaattatggaagatgagattgatgatacggcttaccaggtacattttgaggccaaacagacttggaaaccggtgatagagacagggggaaatgaaagaggtaagttcaccctttcagtccctcagaatctgagcgctctacaacttctccagtgggagacagggattcatgcattggcggagagacttgggggatgcagactgctgcagatcagcacacggggaacgagggatgggatagagttcactgtaagagaaacgccttgcgtttcacctgcttctgatccaatcccttccacatccagatccagttcaattgcttccaatgtctctaccagacaaaccgaatcacctggctccaaatccaatacaagtctcgggataccagaagtcccggctaatttgatcgacataggagcaattgataaagaattcatcctggcagctatctctccgtctgacccaccttataagaacactctgagaaacctcttcgggtccggagattcctttgaacagtacaaccagacaggaatttactctcttaaggaactcgtcgttgctggattgaagagaaaaggaatttataatagaatcagaatcagatgtcatcttgagccccagttcaattga

SEQ ID NO: 24 - RNA, 6279 nt, synthetic construct
  source 1..6279: mol_type = other RNA
atggatctcaacccggtcgatgatgctgcagagttatcagaggagaacttcttctctgggaaattgtccaaggaatgccgaattagaggattgaattcagttgattacaatctgaactccccgttggtgtccgacgatttgacatacttgctgaacaaatttaagggaaaacctgtgcctatccgatggaagatgaagaaatgggactctattctagaccaactgcgtaaacatgatctggagtatctgagaccctcggatctacaccaatggtttgcagaatggatgttgtactcaaagcatggcagtaagcagggagaagacttcttaaagacagtagacgaagaggctagtgatacgtttgaggttgtaaggtcgttcataaggggatggactggtggagaaatcaactttgtcaggaagagcggaaagcatatggggtattgtgcggagttgtgccagaaattcttggatttgcataaattgaccctccttggaaatgccgccaccgataatgagttgttacaactgagcaaaactttcggggatgacaagatttacaagaaacggctaatcaaacttccaagtcttggtcgcgtcatctttgattctggattcttcattgtcctggatcagcgagttctaatggatcgtaattttatgttgatgatgaaagacgtaataattggaagaatgcaaacagttctgtccatgattagcagatgtgatgataaattcagttccaaggatattgattttctattaaaggtctactcaacaggcgataagatcataagaaagttgggtaacgatgggtatgagttgatcaagaccgtagaacctatgtgcaatcttcgtctatcagacctggccagaaggttccgtccactcgtacctcctttccctcatttcaggagacatattgaatcgactgtagatgaactgagtgcgaagactccattgatcagggaattgttctcattgattgatacttctcctaatgtagattcaacgttggtcgtttacggttcatttcgtcattggggtcatccttttattaattattttgaaggcctggagaaattgcataagcaagtgacaatggaaaaagaaattgataccaattattcggaagcgttggccagtgatctggctaggatagtcttgactaaagagttcaatgagaaaaaacaatgggctgtagattaccacagagtgcctacaagccatccctttaaaaaccacatccgagacaacacatggccaacagctgcagtgatccaagattttggagatcactggcatgagctcccattaattcaatgcttcgacatacctgatctgattgatccatccattatctactcagacaagagccattccatgaatcgatcagaggtgttaaatcatgtaagaaccaagcctcatacaccgattccaagcaagaaagttttagagagtatgatcgacaaaccagcgacaaattggcttgagtttcttgaagagatcgacaaaaatggattatctgatgaggatttggtgatcggattaaaaggaaaagaaagagaactgaagattgcaggtagatttttctcattgatgtcttggaagcttagggaatactttgttgtcacagagtatttgatcaagactcactttgtgccattgttccatgggctgaccatggctgatgatatgacagcagtgatcaaaaagatgctggaaagctcgtctggtcaagggctaacaaactacgacagcgtttgtattgcaaatcatattgactatgaaaaatggaataatcatcaacgaaaattgtccaatggacctgtgtttaaagtcatgggacaattcttgggttatccaaatttgatataccggacacatgagttttttgagaaaagtttaatttactataatgaaagaccggatctcatgaaagtcagaaatgggatcttagaaaatagcacacatcagagggtttgctggaatggacaggcaggtggtttagaaggtttgagacagaaaggatggagtattcttaatttgctggtcatacagagagaagcgaaaatccggaataccgcagttaaagtgctagcacaaggcgacaatcaagtcatatgtacacagtacaagaccaaacagtatcgaaacgatatcgagttgagacaagccttaaaccaaatggcagcaaacaatgatgtcatcatgaaggccatagagtcaggaaccaataagttgggtttgttgatcaatcaggatgagacgatgcaatctgctgattatctgaactatgggaaggtacctatctttagaggagtaatcagagggctggaaacaaagcgatggtcccgagtcacttgtgtgacaaatgaccaattaccaacgtgtgcaaatctcatgtcatctgtatctacaaatgctttgacagtggcacatttcgatgtacacccattaaatgcaatgatccaatttaacttcttcgggaattttgctcgccttctcttgattatgcacgatccggcgattcgacaatcattaaatcagttgaaaggaccaaacatcaatgtccattcatacgggttcaaagttgcgatgttgtacttggacccttcaataggaggagtgtgtggtacagccctttctcgatttttgattaggagtttccctgaccctgtgacggagagtctctcattttggaagcttatccatcatagcaccagtgatatcagattaaaaaatctctcagaacagtttggcaatcctaagatagcagtttttagagaatctcacatagaaaagttgttggaagatcctactagtttgaatatatcgatgggaatgagtgctgcaaatttgttgaaaacagagataaagaaaaatctcctacaaaagaaatcaagcatagggaatcagatagtcaaagacgccgtatactacatacactcggaagatgagaaattgagaacttttttgtggtccattactccattgtttcctcggtttctgagcgaatttaaagcagggacgttcatgggagtagcgagcagcattgtatctttgttccaaaactctcgaaccattcgaaatgttttcagagactatatgagtcaaacaattgatgacttgattgtcaagagtgaactaacatctctggagcatttgtctaactatactgacaggaaagggagtggtgggatctggagttgctctgcggaacaggctgacaaacttagaagaatgtcctggaagcgtccagtgctagggacaacagtccctcacccgttggaaatgcacggaaggggaacactgaaatccccgctatccaagtgttgcaaggagtcccgaatggattatatttctgtccatattcctgaaggattgaacaaagttttggacggccgaggaagcctaccagcatatttgggttcaaaaacatcagagtctacctcaattcttcagccttgggagaaggaaagcaaaattcccatcataagaagagctactagattacgagatgcaattcattggtttgtggatccggacagtaacttagcccgaagcatattgaacaatattgaatccttaactggagaaaagtgggaaggggctttaaaaggatataagagaacgggttctgccctccatagattttccacctcgcgggtaagccatggtggtttttcttcacaaagtcctgcatgtctcaccagaatgatggcaaccacagacaccatgcgcgattatgctcagctcaattatgacttcatgttccaagcaagtcttctctactctcaaatgaccagttccgtcattttaatgggaaccactgtctcaaacactattcatttccatgttacctgtcgaaaatgtatccgagaaataacagaaccgatgttagaaagtcctagggagtacagagggaaggacgttcatttagtcctggcaaaatggaagaattcttcaaatggttggggagaaaccctccaactactaaagccagttgagggagattgggacactatccctcctgttgaaaaatcttatcatgtgggtaggatcttgggttttctttatggtgacctaaagagtcaaaattccagtcgtgcagacgacagttcaatcttccccctcagcattcaaatgagattaagaggtcgaggctttttacgaggaatccttgacggattagtccgagctagtgcttgtcaggttatccacaggagaagtgttgctttgctttcaaaaccagcaaatgcaatttatggaggattgatttacttgatagacaagataagtgcatccacctcgtttacgacactgtgtagggacggacccattagagaagaactatcatctatacctcacaaaattcctacttcgtatccaacttcaaattcagatatgggactccacattagaaattatttgaagtttcagtgcaagacagtcgaattgggaaaatatcaatctgatataaaggatttatggttgttctcggatgtgatgacgtcgaatattgcaggaccctttgcattgtctacgaaaattttgaagtgcttatacaagcctgctttgtcccagaaagatcgaaacaatatcaggaagatcagcaatttctccaagatgatgcgatcacaattgagctgggaccccacaagctctgaatttattacatctcaaatcctagtatgcaatgaggaaattaggcatgcctgcaagtttggaattccaaaattatcacttaagtttgatgatccagtctggggacccgaggattatggattgatctggtccattccagttgattattccagtcaatcagttccaaagaacttgaagccctgtcctagaattcaaaaccctagcatatctggttttaggttaggacaacttccaacaggagctcactacaagctgagatcaatcctaaggaagaaaaatatacattacagggatgcactgtgtgggggtgatgggtcaggaggtatgacagcggcagtcctacgttataatctaaaggccagggccatatttaatagcatccttgattttgatggatcaacaatgaagggagcttcaccagatccacctagtgcattggaaacagtggtgaatggccggactcggtgcgtcaacgcggagagttgttgggagaacccgtcagatctgagtgaacagagaacatgggattatttcaaattcttgaaaacccatcacggattaaagatagacctgattgtattggacatggaagtgagagactttgcaatctcggcatccatagagaagtgtgtccgaaacaatgtatcgtcaatattggaagaagacggagtcctaatatacaagacctatggatcaacaattgcagcagaaagtagcaatgcagttgtaaatattggagtattgtttgagagcgtagagttgattcagacggagtatagcagtacatccacttcagaagtgtacatgtattgccgaaagattaagaagtttgtggacgctcctcacccagattggatatcacttgattattattggagcaaattattttgctttaggagttacaaagaagaattcttccgatcctacgaagtgtctcgtaaagaatccctaaaaggaatacccaattccttcatccctgacccattggtcaatctagagacattacttcagatagcaggagttccttctgggatatcacaccagttggcaattgatattaaggaatcacagctgactcagatcaccgctgcaatggtgctttgcggaatgatcgcaaactacactttggatgtgacaaaaaagagagattcatacaatcctccttccgacggaagattaattcgaatgagtgctgctttagtcggaataagtttctggatttccgtcaagtattatgacaaagagttgaatttcgaattggaacagatattatccagatcatttcccataaggtggatgctttcacgcaactatttattctggacaacaaaaaagggttttaggaatgcaaaagacgtaagactatctgggaatatggctaatatagggaattggattcgatgcatggagctccttcatctccccccgggatccctgtccaaggatgaggtcacgaccacttgtgggaaatacattagaagcttgaagtattctgtcatactccaacagaccgggatcattgatttgtggaaatcaagggtcgcaagtgatgatcgatcattgatggaggttaagaccgaattcattgaatctgagcattgggtagattaa

SEQ ID NO: 25 - protein, 450 aa, synthetic construct
MDADKIVFKVNNQVVSLKPEIIVDQYEYKYPAIKDLKKPCITLGKAPDLNKAYKSVLSGMSAAKLNPDDVCSYLAAAMQFFEGTCPEDWTSYGIVIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIMMNGGRLKRSHIRRYVSVSSNHQARPNSFAEFLNKTYSSDS

SEQ ID NO: 26 - protein, 297 aa, synthetic construct
MSKIFVNPSAIRAGLADLEMAEETVDLINRNIEDNQAHLQGEPIEVDNLPEDMGRLHLDDGKSPNHGEIAKVGEGKYREDFQMDEGEDPSFLFQSYLENVGVQIVRQMRSGERFLKIWSQTVEEIISYVAVNFPNPPGKSSEDKSTQTTGRELKKETTPTPSQRESQSSKARMAAQIASGPPALEWSATNEEDDLSVEAEIAHQIAESFSKKYKFPSRSSGILLYNFEQLKMNLDDIVKEAKNVPGVTRLAHDGSKLPLRCVLGWVALANSKKFQLLVESDKLSKIMQDDLNRYTSC

SEQ ID NO: 27 - protein, 2127 aa, synthetic construct
MLDPGEVYDDPIDPIELEAEPRGTPIVPNILRNSDYNLNSPLIEDPARLMLEWLKTGNRPYRMTLTDNCSRSFRVLKDYFKKVDLGSLKVGGMAAQSMISLWLYGAHSESNRSRRCITDLAHFYSKSSPIEKLLNLTLGNRGLRIPPEGVLSCLERVDYDNAFGRYLANTYSSYLFFHVITLYMNALDWDEEKTILALWKDLTSVDIGKDLVKFKDQIWGLLIVTKDFVYSQSSNCLFDRNYTLMLKDLFLSRFNSLMVLLSPPEPRYSDDLISQLCQLYIAGDQVLSMCGNSGYEVIKILEPYVVNSLVQRAEKFRPLIHSLGDFPVFIKDKVSQLEETFGPCARRFFRALDQFDNIHDLVFVFGCYRHWGHPYIDYRKGLSKLYDQVHLKKVIDKSYQECLASDLARRILRWGFDKYSKWYLDSRFLARDHPLTPYIKTQTWPPKHIVDLVGDTWHKLPITQIFEIPESMDPSEILDDKSHSFTRTRLASWLSENRGGPVPSEKVIITALSKPPVNPREFLRSIDLGGLPDEDLIIGLKPKERELKIEGRFFALMSWNLRLYFVITEKLLANYILPLFDALTMTDNLNKVFKKLIDRVTGQGLLDYSRVTYAFHLDYEKWNNHQRLESTEDVFSVLDQVFGLKRVFSRTHEFFQKAWIYYSDRSDLIGLREDQIYCLDASNGPTCWNGQDGGLEGLRQKGWSLVSLLMIDRESQIRNTRTKILAQGDNQVLCPTYMLSPGLSQEGLLYELERISRNALSIYRAVEEGASKLGLIIKKEETMCSYDFLIYGKTPLFRGNILVPESKRWARVSCVSNDQIVNLANIMSTVSTNALTVAQHSQSLIKPMRDFLLMSVQAVFHYLLFSPILKGRVYKILSAEGESFLLAMSRIIYLDPSLGGISGMSLGRFHIRQFSDPVSEGLSFWREIWLSSQESWIHALCQEAGNPDLGERTLESFTRLLEDPTTLNIRGGASPTILLKDAIRKALYDEVDKVENSEFREAILLSKTHRDNFILFLISVEPLFPRFLSELFSSSFLGIPESIIGLIQNSRTIRRQFRKSLSKTLEESFYNSEIHGISRMTQTPQRVGGVWPCSSERADQLREISWGRKVVGTTVPHPSEMLGLLPKSSISCTCGATGGGNPRVSVSVLPSFDQSFFSRGPLKGYLGSSTSMSTQLFHAWEKVTNVHVVKRALSLKESINWFITRDSNLAQALIRNIMSLTGPDFPLEEAPVFKRTGSALHRFKSARYSEGGYSSVCPNLLSHISVSTDTMSDLTQDGKNYDFMFQPLMLYAQTWTSELVQRDTRLRDSTFHWHLRCNRCVRPIDDVTLETSQIFEFPDVSKRISRMVSGAVPHFQRLPDIRLRPGDFESLSGREKSHHIGSAQGLLYSILVAIHDSGYNDGTIFPVNIYGKVSPRDYLRGLARGVLIGSSICFLTRMTNININRPLELVSGVISYILLRLDNHPSLYIMLREPSLRGEIFSIPQKIPAAYPTTMKEGNRSILCYLQHVLRYEREIITASPENDWLWIFSDFRSAKMTYLSLITYQSHLLLQRVERNLSKSMRDNLRQLSSLMRQVLGGHGEDTLESDDNIQRLLKDSLRRTRWVDQEVRHAARTMTGDYSPNKKVSRKVGCSEWVCSAQQVAVSTSANPAPVSELDIRALSKRFQNPLISGLRVVQWATGAHYKLKPILDDLNVFPSLCLVVGDGSGGISRAVLNMFPDAKLVFNSLLEVNDLMASGTHPLPPSAIMRGGNDIVSRVIDLDSIWEKPSDLRNLATWKYFQSVQKQVNMSYDLIICDAEVTDIASINRITLLMSDFALSIDGPLYLVFKTYGTMLVNPNYKAIQHLSRAFPSVTGFITQVTSSFSSELYLRFSKRGKFFRDAEYLTSSTLREMSLVLFNCSSPKSEMQRARSLNYQDLVRGFPEEIISNPYNEMIITLIDSDVESFLVHKMVDDLELQRGTLSKVAIIIAIMIVFSNRVFNVSKPLTDPSFYPPSDPKILRHFNICCSTMMYLSTALGDVPSFARLHDLYNRPITYYFRKQVIRGNVYLSWSWSNDTSVFKRVACNSSLSLSSHWIRLIYKIVKTTRLVGSIKDLSREVERHLHRYNRWITLEDIRSRSSLLDYSCL

SEQ ID NO: 28 - RNA, 1353 nt, synthetic construct
  source 1..1353: mol_type = other RNA
atggatgccgacaagattgtattcaaagtcaataatcaggtggtctctttgaagcctgagattatcgtggatcaatatgagtacaagtaccctgccatcaaagatttgaaaaagccctgtataaccctaggaaaggctcccgatttaaataaagcatacaagtcagttttgtcaggcatgagcgccgccaaacttaatcctgacgatgtatgttcctatttggcagcggcaatgcagttttttgaggggacatgtccggaagactggaccagctatggaattgtgattgcacgaaaaggagataagatcaccccaggttctctggtggagataaaacgtactgatgtagaagggaattgggctctgacaggaggcatggaactgacaagagaccccactgtccctgagcatgcgtccttagtcggtcttctcttgagtctgtataggttgagcaaaatatccgggcaaaacactggtaactataagacaaacattgcagacaggatagagcagatttttgagacagccccttttgttaaaatcgtggaacaccatactctaatgacaactcacaaaatgtgtgctaattggagtactataccaaacttcagatttttggccggaacctatgacatgtttttctcccggattgagcatctatattcagcaatcagagtgggcacagttgtcactgcttatgaagactgttcaggactggtatcatttactgggttcataaaacaaatcaatctcaccgctagagaggcaatactatatttcttccacaagaactttgaggaagagataagaagaatgtttgagccagggcaggagacagctgttcctcactcttatttcatccacttccgttcactaggcttgagtgggaaatctccttattcatcaaatgctgttggtcacgtgttcaatctcattcactttgtaggatgctatatgggtcaagtcagatccctaaatgcaacggttattgctgcatgtgctcctcatgaaatgtctgttctagggggctatctgggagaggaattcttcgggaaagggacatttgaaagaagattcttcagagatgagaaagaacttcaagaatacgaggcggctgaactgacaaagactgacgtagcactggcagatgatggaactgtcaactctgacgacgaggactacttttcaggtgaaaccagaagtccggaggctgtttatactcgaatcatgatgaatggaggtcgactaaagagatctcacatacggagatatgtctcagtcagttccaatcatcaagcccgtccaaactcattcgccgagtttctaaacaagacatattcgagtgactcataa

SEQ ID NO: 29 - RNA, 894 nt, synthetic construct
  source 1..894: mol_type = other RNA
atgagcaagatctttgtcaatcctagtgctattagagccggtctggccgatcttgagatggctgaagaaactgttgatctgatcaatagaaatatcgaagacaatcaggctcatctccaaggggaacccatagaggtggacaatctccctgaggatatggggcgacttcacctggatgatggaaaatcgcccaaccatggtgagatagccaaggtgggagaaggcaagtatcgagaggactttcagatggatgaaggagaggatcctagcttcctgttccagtcatacctggaaaatgttggagtccaaatagtcagacaaatgaggtcaggagagagatttctcaagatatggtcacagaccgtagaagagattatatcctatgtcgcggtcaactttcccaaccctccaggaaagtcttcagaggataaatcaacccagactactggccgagagctcaagaaggagacaacacccactccttctcagagagaaagccaatcatcgaaagccaggatggcggctcaaattgcttctggccctccagcccttgaatggtcggctaccaatgaagaggatgatctatcagtggaggctgagatcgctcaccagattgcagaaagtttctccaaaaaatataagtttccctctcgatcctcagggatactcttgtataattttgagcaattgaaaatgaaccttgatgatatagttaaagaggcaaaaaatgtaccaggtgtgacccgtttagcccatgacgggtccaaactccccctaagatgtgtactgggatgggtcgctttggccaactctaagaaattccagttgttagtcgaatccgacaagctgagtaaaatcatgcaagatgacttgaatcgctatacatcttgctaa

SEQ ID NO: 30 - RNA, 6384 nt, synthetic construct
  source 1..6384: mol_type = other RNA
atgctcgatcctggagaggtctatgatgaccctattgacccaatcgagttagaggctgaacccagaggaacccccattgtccccaacatcttgaggaactctgactacaatctcaactctcctttgatagaagatcctgctagactaatgttagaatggttaaaaacagggaatagaccttatcggatgactctaacagacaattgctccaggtctttcagagttttgaaagattatttcaagaaggtagatttgggttctctcaaggtgggcggaatggctgcacagtcaatgatttctctctggttatatggtgcccactctgaatccaacaggagccggagatgtataacagacttggcccatttctattccaagtcgtcccccatagagaagctgttgaatctcacgctaggaaatagagggctgagaatccccccagagggagtgttaagttgccttgagagggttgattatgataatgcatttggaaggtatcttgccaacacgtattcctcttacttgttcttccatgtaatcaccttatacatgaacgccctagactgggatgaggaaaagaccatcctagcattatggaaagatttaacctcagtggacatcgggaaggacttggtaaagttcaaagaccaaatatggggactgctgatcgtgacaaaggactttgtttactcccaaagttccaattgtctttttgacagaaactacacacttatgctaaaagatcttttcttgtctcgcttcaactccttaatggtcttgctctctcccccagagccccgatactcagatgacttgatatctcaactatgccagctgtacattgctggggatcaagtcttgtctatgtgtggaaactccggctatgaagtcatcaaaatattggagccatatgtcgtgaatagtttagtccagagagcagaaaagtttaggcctctcattcattccttgggagactttcctgtatttataaaagacaaggtaagtcaacttgaagagacgttcggtccctgtgcaagaaggttctttagggctctggatcaattcgacaacatacatgacttggtttttgtgtttggctgttacaggcattgggggcacccatatatagattatcgaaagggtctgtcaaaactatatgatcaggttcaccttaaaaaagtgatagataagtcctaccaggagtgcttagcaagcgacctagccaggaggatccttagatggggttttgataagtactccaagtggtatctggattcaagattcctagcccgagaccaccccttgactccttatatcaaaacccaaacatggccacccaaacatattgtagacttggtgggggatacatggcacaagctcccgatcacgcagatctttgagattcctgaatcaatggatccgtcagaaatattggatgacaaatcacattctttcaccagaacgagactagcttcttggctgtcagaaaaccgaggggggcctgttcctagcgaaaaagttattatcacggccctgtctaagccgcctgtcaatccccgagagtttctgaggtctatagacctcggaggattgccagatgaagacttgataattggcctcaagccaaaggaacgggaattgaagattgaaggtcgattctttgctctaatgtcatggaatctaagattgtattttgtcatcactgaaaaactcttggccaactacatcttgccactttttgacgcgctgactatgacagacaacctgaacaaggtgtttaaaaagctgatcgacagggtcaccgggcaagggcttttggactattcaagggtcacatatgcatttcacctggactatgaaaagtggaacaaccatcaaagattagagtcaacagaggatgtattttctgtcctagatcaagtgtttggattgaagagagtgttttctagaacacacgagttttttcaaaaggcctggatctattattcagacagatcagacctcatcgggttacgggaggatcaaatatactgcttagatgcgtccaacggcccaacctgttggaatggccaggatggcgggctagaaggcttacggcagaagggctggagtctagtcagcttattgatgatagatagagaatctcaaatcaggaacacaagaaccaaaatactagctcaaggagacaaccaggttttatgtccgacatacatgttgtcgccagggctatctcaagaggggctcctctatgaattggagagaatatcaaggaatgcactttcgatatacagagccgtcgaggaaggggcatctaagctagggctgatcatcaagaaagaagagaccatgtgtagttatgacttcctcatctatggaaaaacccctttgtttagaggtaacatattggtgcctgagtccaaaagatgggccagagtctcttgcgtctctaatgaccaaatagtcaacctcgccaatataatgtcgacagtgtccaccaatgcgctaacagtggcacaacactctcaatctttgatcaaaccgatgagggattttctgctcatgtcagtacaggcagtctttcactacctgctatttagcccaatcttaaagggaagagtttacaagattctgagcgctgaaggggagagctttctcctagccatgtcaaggataatctatctagatccttctttgggagggatatctggaatgtccctcggaagattccatatacgacagttctcagaccctgtctctgaagggttatccttctggagagagatctggttaagctcccaagagtcctggattcacgcgttgtgtcaagaggctggaaacccagatcttggagagagaacactcgagagcttcactcgccttctagaagatccgaccaccttaaatatcagaggaggggccagtcctaccattctactcaaggatgcaatcagaaaggctttatatgacgaggtggacaaggtggaaaattcagagtttcgagaggcaatcctgttgtccaagacccatagagataattttatactcttcttaatatctgttgagcctctgtttcctcgatttctcagtgagctattcagttcgtcttttttgggaatccccgagtcaatcattggattgatacaaaactcccgaacgataagaaggcagtttagaaagagtctctcaaaaactttagaagaatccttctacaactcagagatccacgggattagtcggatgacccagacacctcagagggttgggggggtgtggccttgctcttcagagagggcagatcaacttagggagatctcttggggaagaaaagtggtaggcacgacagttcctcacccttctgagatgttgggattacttcccaagtcctctatttcttgcacttgtggagcaacaggaggaggcaatcctagagtttctgtatcagtactcccgtcctttgatcagtcatttttttcacgaggccccctaaagggatacttgggctcgtccacctctatgtcgacccagctattccatgcatgggaaaaagtcactaatgttcatgtggtgaagagagctctatcgttaaaagaatctataaactggttcattactagagattccaacttggctcaagctctaattaggaacattatgtctctgacaggccctgatttccctctagaggaggcccctgtcttcaaaaggacggggtcagccttgcataggttcaagtctgccagatacagcgaaggagggtattcttctgtctgcccgaacctcctctctcatatttctgttagtacagacaccatgtctgatttgacccaagacgggaagaactacgatttcatgttccagccattgatgctttatgcacagacatggacatcagagctggtacagagagacacaaggctaagagactctacgtttcattggcacctccgatgcaacaggtgtgtgagacccattgacgacgtgaccctggagacctctcagatcttcgagtttccggatgtgtcgaaaagaatatccagaatggtttctggggctgtgcctcacttccagaggcttcccgatatccgtctgagaccaggagattttgaatctctaagcggtagagaaaagtctcaccatatcggatcagctcaggggctcttatactcaatcttagtggcaattcacgactcaggatacaatgatggaaccatcttccctgtcaacatatacggcaaggtttcccctagagactatttgagagggctcgcaaggggagtattgataggatcctcgatttgcttcttgacaagaatgacaaatatcaatattaatagacctcttgaattggtctcaggggtaatctcatatattctcctgaggctagataaccatccctccttgtacataatgctcagagaaccgtctcttagaggagagatattttctatccctcagaaaatccccgccgcttatccaaccactatgaaagaaggcaacagatcaatcttgtgttatctccaacatgtgctacgctatgagcgagagataatcacggcgtctccagagaatgactggctatggatcttttcagactttagaagtgccaaaatgacgtacctatccctcattacttaccagtctcatcttctactccagagggttgagagaaacctatctaagagtatgagagataacctgcgacaattgagttctttgatgaggcaggtgctgggcgggcacggagaagataccttagagtcagacgacaacattcaacgactgctaaaagactctttacgaaggacaagatgggtggatcaagaggtgcgccatgcagctagaaccatgactggagattacagccccaacaagaaggtgtcccgtaaggtaggatgttcagaatgggtctgctctgctcaacaggttgcagtctctacctcagcaaacccggcccctgtctcggagcttgacataagggccctctctaagaggttccagaaccctttgatctcgggcttgagagtggttcagtgggcaaccggtgctcattataagcttaagcctattctagatgatctcaatgttttcccatctctctgccttgtagttggggacgggtcaggggggatatcaagggcagtcctcaacatgtttccagatgccaagcttgtgttcaacagtcttttagaggtgaatgacctgatggcttccggaacacatccactgcctccttcagcaatcatgaggggaggaaatgatatcgtctccagagtgatagatcttgactcaatctgggaaaaaccgtccgacttgagaaacttggcaacctggaaatacttccagtcagtccaaaagcaggtcaacatgtcctatgacctcattatttgcgatgcagaagttactgacattgcatctatcaaccggatcaccctgttaatgtccgattttgcattgtctatagatggaccactctatttggtcttcaaaacttatgggactatgctagtaaatccaaactacaaggctattcaacacctgtcaagagcgttcccctcggtcacagggtttatcacccaagtaacttcgtctttttcatctgagctctacctccgattctccaaacgagggaagtttttcagagatgctgagtacttgacctcttccacccttcgagaaatgagccttgtgttattcaattgtagcagccccaagagtgagatgcagagagctcgttccttgaactatcaggatcttgtgagaggatttcctgaagaaatcatatcaaatccttacaatgagatgatcataactctgattgacagtgatgtagaatcttttctagtccacaagatggttgatgatcttgagttacagaggggaactctgtctaaagtggctatcattatagccatcatgatagttttctccaacagagtcttcaacgtttccaaacccctaactgacccctcgttctatccaccgtctgatcccaaaatcctgaggcacttcaacatatgttgcagtactatgatgtatctatctactgctttaggtgacgtccctagcttcgcaagacttcacgacctgtataacagacctataacttattacttcagaaagcaagtcattcgagggaacgtttatctatcttggagttggtccaacgacacctcagtgttcaaaagggtagcctgtaattctagcctgagtctgtcatctcactggatcaggttgatttacaagatagtgaagactaccagactcgttggcagcatcaaggatctatccagagaagtggaaagacaccttcataggtacaacaggtggatcaccctagaggatatcagatctagatcatccctactagactacagttgcctgtga

SEQ ID NO: 31 - protein, 540 aa, synthetic construct
MEKPVVNVDVDPQSPFVVQLQKSFPQFEVVAQQVTPNDHANARAFSHLASKLIELEVPTTATILDIGSAPARRMFSEHQYHCVCPMRSPEDPDRMMKYASKLAEKACKITNKNLHEKIKDLRTVLDTPDAETPSLCFHNDVTCNMRAEYSVMQDVYINAPGTIYHQAMKGVRTLYWIGFDTTQFMFSAMAGSYPAYNTNWADEKVLEARNIGLCSTKLSEGRTGKLSIMRKKELKPGSRVYFSVGSTLYPEHRASLQSWHLPSVFHLNGKQSYTCRCDTVVSCEGYVVKKITISPGITGETVGYAVTHNSEGFLLCKVTDTVKGERVSFPVCTYIPATICDQMTGIMATDISPDDAQKLLVGLNQRIVINGRTNRNTNTMQNYLLPIIAQGFSKWAKERKDDLDNEKMLGTRERKLTYGCLWAFRTKKVHSFYRPPGTQTCVKVPASFSAFPMSSVWTTSLPMSLRQKLKLALQPKKEEKLLQVSEELVMEAKAAFEDAQEEARAEKLREALPPLVADKGIEAAAEVVCEVEGLQADIGA

SEQ ID NO: 32 - protein, 807 aa, synthetic construct
ALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQPTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVKKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGA

SEQ ID NO: 33 - protein, 549 aa, synthetic construct
APSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEY

SEQ ID NO: 34 - protein, 557 aa, synthetic construct
MMPTEANKSRYQSRKVENQKAITTERLLSGLRLYNSATDQPECYKITYPKPLYSSSVPANYSDPQFAVAVCNNYLHENYPTVASYQITDEYDAYLDMVDGTVACLDTATFCPAKLRSYPKKHEYRAPNIRSAVPSAMQNTLQNVLIAATKRNCNVTQMRELPTLDSATFNVECFRKYACNDEYWEEFARKPIRITTEFVTAYVARLKGPKAAALFAKTYNLVPLQEVPMDRFVMDMKRDVKVTPGTKHTEERPKVQVIQAAEPLATAYLCGIHRELVRRLTAVLLPNIHTLFDMSAEDFDAIIAEHFKQGDPVLETDIASFDKSQDDAMALTGLMILEDLGVDQPLLDLIECAFGEISSTHLPTGTRFKFGAMMKSGMFLTLFVNTVLNVVIASRVLEERLKTSRCAAFIGDDNIIHGVVSDKEMAERCATWLNMEVKIIDAVIGERPPYFCGGFILQDSVTSTACRVADPLKRLFKLGKPLPADDEQDEDRRRALLDETKAWFRVGITGTLAVAVTTRYEVDNITPVLLALRTFAQSKRAFQAIRGEIKHLYGGPK

SEQ ID NO: 35 - RNA, 1620 nt, synthetic construct
  source 1..1620: mol_type = other RNA
atggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagca

SEQ ID NO: 36 - RNA, 2421 nt, synthetic construct
  source 1..2421: mol_type = other RNA
gcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagcc

SEQ ID NO: 37 - RNA, 1647 nt, synthetic construct
  source 1..1647: mol_type = other RNA
gcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatac

SEQ ID NO: 38 - RNA, 1674 nt, synthetic construct
  source 1..1674: mol_type = other RNA
atgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatag

SEQ ID NO: 39 - DNA, 1269 bp, synthetic construct
  source 1..1269: mol_type = other DNA
atgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatga

SEQ ID NO: 40 - DNA, 798 bp, synthetic construct
  source 1..798: mol_type = other DNA
atggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtag

SEQ ID NO: 41 - DNA, 6330 bp, synthetic construct
  source 1..6330: mol_type = other DNA
atggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaa

SEQ ID NO: 42 - protein, 386 aa, synthetic construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFRDEDTQAMPFRVTERESKPVQMMYQTGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

(19) United States
(12) Patent Application Publication
(10) Pub. No.: US 2024/0401076 A1
(43) Pub. Date: Dec. 5, 2024

(54) SELF-REPLICATING RNA MOLECULES AND APPLICATION THEREOF

(71) Applicant: R&R THERAPEUTICS INC., Shanghai (CN)

(72) Inventor: Kexing WU, Shanghai (CN)

(21) Appl. No.: 18/630,513

(22) Filed: Apr. 9, 2024

Related U.S. Application Data
(63) Continuation of application No. PCT/CN2022/124192, filed on Oct. 9, 2022.

(30) Foreign Application Priority Data
Oct. 9, 2021 (CN) 202111178106.7

(51) Int. Cl. C12N 15/86; C07K 14/005

(52) U.S. Cl. CPC C12N 15/86; C07K 14/005; C12N 2760/20122; C12N 2760/20222; C12N 2760/20242; C12N 2760/20243

(57) ABSTRACT

Provided are a self-replicating RNA molecule and uses thereof. The self-replicating RNA molecule includes: a first RNA sequence encoding an N protein or a functional fragment of the N protein, a second RNA sequence encoding a P protein or a functional fragment of the P protein, and a third RNA sequence encoding an L protein or a functional fragment of the L protein. The self-replicating RNA molecule is suitable for producing the N protein, the P protein, the L protein, or the functional fragments thereof in animal cells.

### CROSS-REFERENCE TO RELATED APPLICATIONS

This application a continuation of International Application No. PCT/CN2022/124192, filed on Oct. 9, 2022, which claims the priority and benefit of the patent application Ser. No. 20/211,1178106.7 filed with China National Intellectual Property Administration on Oct. 9, 2021. The disclosures of the aforementioned applications are hereby incorporated by reference in their entireties.

### STATEMENT REGARDING SEQUENCE LISTING

A Sequence Listing associated with this application is being filed concurrently herewith and is hereby incorporated by reference into the present specification. The text file containing the Sequence listing is titled “Sequence_Listing.xml”, was created on Aug. 23, 2024, and is 109 kilobytes in size.

### FIELD

The present disclosure relates to the field of biomedicine, and particularly, to the design of self-replicating RNA molecules and uses thereof.

### BACKGROUND

The development of mRNA-based vaccines and therapeutics has become one of the hot spots in recent years. The mechanism of mRNA, when serving as vaccine or using for therapy, is to stimulate the immune system to respond, or to express proteins for treating. Therefore, the protein expression level is closely related to the therapeutic effect. However, as one of the disadvantages, mRNA is not very stable and may degrade within a few days in cells, resulting in an unsustainable protein expression level. If it is used for disease treatment in a long period, patients have to be injected with a large amount of mRNA, which may increase the toxic side effects of mRNA therapy.

Therefore, at present, mRNA-based vaccines or therapeutics still need to be improved.

### SUMMARY

The present disclosure aims to solve one of the technical problems in the related art at least to some extent.

Through in-depth study on the mechanism of translation and self-amplification of RNA of various RNA viruses in cells, the Applicant found that: by using RNA molecules encoding N, P and L proteins derived from Rhabdovirus as the core region, RNA can be self-replicated and translated in animal cells, and the core region, as a powerful “engine”, can provide “kinetic energy” for high-efficient transcription amplification and for activating macromolecular proteins. The core region can further carry a “loading region” to replicate or translate the target molecules, which cover almost all protein medicaments available on the market. According to the embodiments of the present disclosure, the “loading region” can be designed with different protein coding boxes to enable the organism to produce various peptides, enzymes, antibodies, channel proteins, receptor proteins and the like in cells, thereby achieving different therapeutic purposes, covering tumor pipelines, vaccine pipelines, rare disease pipelines and prospective general product pipelines. Therefore, the Applicant provides a novel self-replicating RNA molecule, which is referred to as reRNA™. As mentioned above, reRNA™ includes an element coding sequence capable of implementing RNA translation and self-amplification, and a carried gene sequence encoding the target molecule.

Based on the above, in a first aspect of the present disclosure, the present disclosure provides a self-replicating RNA molecule. The self-replicating RNA molecule includes a first RNA sequence, encoding an N protein or a functional fragment of the N protein, a second RNA sequence, encoding a P protein or a functional fragment of the P protein, and a third RNA sequence, encoding an L protein or a functional fragment of the L protein. The self-replicating RNA molecule is suitable for producing the N protein, the P protein, the L protein, or the functional fragments thereof in animal cells. Referring to FIG. 1, the first RNA sequence, the second RNA sequence, and the third RNA sequence constitute a “core region” of the self-replicating RNA molecule, also referred to as “core life region”, which can serve as the smallest region capable of implementing self-replication and translation in animal cells. The core region can further carry a loading region configured to carry other encoding or non-encoding sequences for expressing or replicating target molecules and expressing small molecules, various peptides, enzymes, antibodies, channel proteins, receptor proteins and the like, thereby achieving various therapeutic purposes, covering tumor pipelines, vaccine pipelines, rare disease pipelines and prospective general product pipelines.

In a second aspect of the present disclosure, the present disclosure provides a DNA molecule, encoding the self-replicating RNA molecule described in any one of the preceding items.

In a third aspect of the present disclosure, the present disclosure provides an expression vector, carrying the DNA molecule as mentioned above.

In a fourth aspect of the present disclosure, the present disclosure provides a method for preparing the self-replicating RNA molecule as mentioned above. The method includes: expressing, using the DNA molecule or expression vector as mentioned above, the self-replicating RNA molecule in host cells, and collecting the self-replicating RNA molecule.

In a fifth aspect, the present disclosure provides an RNA-protein complex. The RNA-protein complex includes the self-replicating RNA molecule as mentioned above, and a protein including the N protein or functional fragment thereof, the P protein or functional fragment thereof, and the L protein or functional fragment thereof.

In a sixth aspect of the present disclosure, the present disclosure provides a pharmaceutical composition. The pharmaceutical composition includes the self-replicating RNA molecule, DNA molecule, expression vector, or RNA-protein complex as mentioned above.

In a seventh aspect, the present disclosure provides a method for expressing a target molecule in an individual. The method includes administering to the individual the self-replicating RNA molecule, DNA molecule, expression vector, RNA-protein complex, or pharmaceutical composition as mentioned above.

Additional aspects and advantages of the present disclosure will be set forth in part in the following description, and in part will be obvious from the following description, or may be learned by practice of the present disclosure.

### BRIEF DESCRIPTION OF THE DRAWINGS

The above and/or additional aspects and advantages of the present disclosure will be apparent and easily understood from the description of the embodiments taken in conjunction with the following figures, in which:

FIG. 1 is a structural schematic diagram of a self-replicating RNA molecule (reRNA™) according to an embodiment of the present disclosure,

FIG. 2 is a structural schematic diagram of a protein-RNA complex containing the self-replicating RNA molecule according to an embodiment of the present disclosure,

FIG. 3, FIG. 5, and FIG. 6 respectively show expression results of green fluorescent protein according to Example 1, Example 3, and Example 4 of the present disclosure,

FIG.4 shows RNA self-replication results of green fluorescent protein according to Example 2 of the present disclosure,

FIG. 7 shows detection results of OVA antigen presentation assay on antigen-presenting cells by flow cytometry according to Example 8 of the present disclosure.

### DETAILED DESCRIPTION

Embodiments of the present disclosure are described in detail below. Examples of which are illustrated in the accompanying figures. The embodiments described below by referring to the accompanying figures are exemplary and are intended to explain the present disclosure, but not to be construed as limitations of the present disclosure.

As used herein, the terms “include”, “including”, “comprise”, “comprising” are open expressions, that is, indicating the inclusion of the contents specified in the present disclosure, while not excluding other aspects.

As used herein, the term “optionally” or “optional” generally means that the event or situation described subsequently may but not necessarily occur, and the description includes the situation in which the event or situation occurs and the situation in which the event or situation does not occur.

### Self-Replicating RNA Molecule

As used herein, the term “self-replicating RNA molecule” may also be referred to as “self-amplifying RNA”. The self-replicating RNA molecule differs from common mRNA mainly in that the self-replicating RNA molecule uses its own RNA sequence as a template for self-replication. According to the embodiments of the present disclosure, the self-replicating RNA molecules can be translated and replicated in cytoplasm even without entering the nucleus, thereby avoiding the potential risks caused by integration with the genome. Generally, mRNA encodes the protein to be expressed, and the ribosomes in cells are used to complete the translation and protein production. According to the embodiments of the present disclosure, the self-replicating RNA molecule carries a sequence capable of expressing RNA polymerase (RNA-dependent RNA polymerase), and after the RNA molecule is translated into RNA polymerase in cytoplasm, more self-replicating RNA molecules can be generated by using the self-replicating RNA molecule as a template.

Through in-depth study on the mechanism of translation and self-amplification of RNA of various RNA viruses in cells, the Applicant found that, when the RNA molecule encoding N protein, P protein and L protein derived from Rhabdovirus is used as a core region, or when the RNA molecule encoding NSP1, NSP2, NSP3 and NP4 proteins derived from positive-strand RNA viruses as the core region, self-replication and translation of RNA can be achieved in animal cells. The core region, as a powerful “engine”, can provide “kinetic energy” for high-efficient transcription amplification and for activating macromolecular proteins. The core region can further carry a “loading region” to replicate or translate the target molecules, which cover almost all protein medicaments available on the market. According to the embodiments of the present disclosure, the “loading region” can be designed with different protein coding boxes to enable an organism to produce various peptides, enzymes, antibodies, channel proteins, receptor proteins and the like in cells, thereby achieving different therapeutic purposes, covering tumor pipelines, vaccine pipelines, rare disease pipelines and prospective general product pipelines. Therefore, the Applicant provides a novel self-replicating RNA molecule, which is referred to as reRNA™ As mentioned above, reRNA™ includes an element coding sequence capable of implementing RNA translation and self-amplification, and a carried gene sequence encoding the target molecule. According to the embodiments of the present disclosure, reRNA™, by taking the characteristics of RNA virus into consideration, can achieve the in vivo amplification from RNA to mRNA and then from mRNA to protein, thereby expressing therapeutic medicaments with high efficiency and long-term effect to achieve better clinical efficacy.

The mechanism of mRNA for treatment is to stimulate a respond of the immune system or express proteins. Therefore, the protein expression level is closely related to the therapeutic effect. However, as mentioned above, the conventional mRNA is not stable enough, and it may degrade within a few days in cells, resulting in unsustainable protein expression level. If mRNA is used for long-period disease treatment, patients may be required to be injected with a large amount of mRNA, which may intensify the toxic side effects of mRNA therapy. The self-amplify RNA at a very low dose can achieve the same protein expression level as conventional mRNA, as it can self-replicate in the cytoplasm. For example, the amount of self-replicating RNA molecules can be hundreds or even thousands of times smaller than that of conventional messenger RNA, while exerting the same immune stimulating effect and protein biological activity. The dosage and times of injection in drug therapy or immune stimulation can be reduced, thereby prolonging the therapeutic effect and reducing the potential toxic side effects of medicaments and drug delivery carriers. In addition, the self-replicating RNA molecules may form double-stranded RNA during replication, which is very similar to viral RNA during the replication process, and thus it may stimulate the innate immune response of cells and further enhance the effect of the vaccines.

The Applicant found in the research the following fact. At least the sequence encoding RNA polymerase is linked to the sequence expressing the target protein by the self-replicating RNA molecule, and thus the molecular weight of the whole mRNA molecule is much greater than that of the conventional mRNA. The excessively great molecular weight may lead to a significant decrease in delivery efficiency, translation efficiency, and replication efficiency. In order to improve these efficiencies, the Applicant conducted in-depth research, in order to find the shortest nucleic acid fragment capable of normally exerting the functions of self-replication and translation.

In an aspect of the present disclosure, the present disclosure provides a self-replicating RNA molecule. The self-replicating RNA molecule includes a first RNA sequence, a second RNA sequence, and a third RNA sequence. The first RNA sequence encodes an N protein or a functional fragment thereof. The second RNA sequence encodes a P protein or a functional fragment thereof. The third RNA sequence encodes an L protein or a functional fragment thereof. Alternatively, the self-replicating RNA molecule includes a fourth RNA sequence, and the fourth RNA sequence encodes an Nsp protein or a functional fragment thereof. The self-replicating RNA molecule is suitable for producing the N protein, the P protein, the L protein, or the functional fragments thereof in animal cells, or the self-replicating RNA molecule is suitable for producing the Nsp protein or the functional fragment thereof in animal cells. Therefore, the first, second and third RNA sequences, or the fourth RNA sequence, constitute the “core region” of the self-replicating RNA molecule, also referred to as the “core life region”. The core region can serve as the smallest region capable of implementing self-replication and translation in the animal cells. The core region can further carry other encoding sequences to express or replicate the target molecules, thereby exerting the functions of medicaments or vaccines.

The term “functional fragment” as used herein refers to a part of the full-length sequence of a protein, but this part can still play a function related to the self-replication of RNA molecules. For example, the functional fragment may be a truncated protein of the full-length sequence, or a protein of a full-length sequence in which the amino acid sequence has undergone changes such as substitutions, mutations, or deletions. According to the embodiments of the present disclosure, the functional fragment of the N protein can bind to RNA molecules to protect RNA from being affected by nuclease. The functional fragment of the P protein can bind to the N protein to locate L polymerase on the template, and it can also be used as a basic component of RNA polymerase transcription and replication complex. Further, the functional fragment of L protein can play the role of RNA polymerase, which is related to RNA transcription and replication.

According to the embodiments of the present disclosure, the biggest technical difficulty how is to simultaneously achieve the translation of RNA and self-amplification in cells. With the existing mRNA technology, the translation of RNA can be achieved in cells, but the self-amplification of RNA cannot be implemented. In order to solve this problem, the Applicant investigated the mechanism of RNA translation and self-amplification of various RNA viruses in cells to find a protein or protein complex capable of implementing the translation and self-amplification of RNA in cells. Through a lot of research work, the Applicant finally confirmed that nucleocapsid protein complex of Rhabdovirus was the optimal candidate combination. The nucleocapsid protein complex of Rhabdovirus contains N protein, P protein, and L protein. In order to determine the smallest functional unit, the Applicant used the nucleocapsid protein complex protein of vesicular stomatitis virus (VSV, Indiana) for screening. The results reveal that the N protein, P protein and L protein cannot individually implement the translation and self-amplification of reRNA™ in cells, and the combination of two proteins of NP, NL and PL also cannot implement the translation and self-amplification of reRNA™ in cells. A complex of three proteins NPL can achieve the translation and self-amplification of reRNA™ in cells. It should be noted that such a combination includes, but is not limited to, the NPL protein combinations of Vesiculorius, Lyssavirus, Ephemeroviru, non-virulent Rhabdovirus, etc. of Rhabdoviridae, and the cross-combination. In this combination, the N protein functions as an RNA-binding protein for protecting RNA from being affected by nuclease. The P protein functions as a linker protein to bind to the N protein, and locates L polymerase on the template. The P protein is also a basic component of RNA polymerase transcription and replication complex. The L protein functions as an RNA polymerase and is related to RNA transcription and replication. Those skilled in the art can understand that the N protein, P protein and L protein may be derived from different types of viruses.

According to the embodiments of the present disclosure, at least one of the N protein, the P protein, and the L protein is independently derived from Rhabdoviridae virus. According to the embodiments of the present disclosure, the Rhabdoviridae virus includes at least one virus selected from Vesiculorius, Lyssavirus, and Ephemerovirus. Vesiculorius includes, but is not limited to, Vesicular stomatitis New Jersey virus, Maraba virus, Vesicular stomatitis Alagoas virus Indiana 3, Cocal virus Indiana 2, Isfahan virus, Chandipura virus, Spring viraemia of carp virus, American bat vesiculovirus, Carajas vesiculovirus, Jurona vesiculovirus, Malpais Spring vesiculovirus, Morreton vesiculovirus, Perinet vesiculovirus, Piry vesiculovirus, Radi vesiculovirus, and Yug Bogdanovac vesiculovirus. Lyssavirus includes, but is not limited to, West Caucasian bat virus. Aravan lyssavirus, Khujand lyssavirus, Rabies virus, Australian bat lyssavirus, European bat lyssavirus 1, European bat lyssavirus 2, Irkut virus, Shimoni bat virus, Lagos bat virus, Mokola virus, Lleida bat lyssavirus, Ikoma lyssavirus, Gannoruwa bat lyssavirus, Bokeloh bat lyssavirus, Duvenhage lyssavirus. Ephemerovirus includes, but is not limited to, Bovine fever ephemerovirus, Adelaide River ephemerovirus, Berrimah ephemerovirus, and Kimberley ephemerovirus.

Thus, according to the embodiments of the present disclosure, Rhabdoviridae virus includes at least one virus selected from Vesicular stomatitis New Jersey virus, Maraba virus, Vesicular stomatitis Alagoas virus Indiana 3, Cocal virus Indiana 2, Isfahan virus, Chandipura virus, Spring viraemia of carp virus, American bat vesiculovirus, Carajas vesiculovirus, Jurona vesiculovirus, Malpais Spring vesiculovirus, Morreton vesiculovirus, Perinet vesiculovirus,

Piry vesiculovirus, Radi vesiculovirus, Yug Bogdanovac vesiculovirus, West Caucasian bat virus. Aravan lyssavirus, Khujand lyssavirus, Rabies virus, Australian bat lyssavirus, European bat lyssavirus 1, European bat lyssavirus 2, Irkut virus, Shimoni bat virus, Lagos bat virus, Mokola virus, Lleida bat lyssavirus, Ikoma lyssavirus, Gannoruwa bat lyssavirus, Bokeloh bat lyssavirus, Duvenhage lyssavirus, Bovine fever ephemerovirus, Adelaide River ephemerovirus, Berrimah ephemerovirus, and Kimberley ephemerovirus

According to the embodiments of the present disclosure, the N protein, the P protein, and the L protein may also be each independently derived from different virus serotypes of the same virus species, for example, including, but not limited to, Indiana strain, New Jersey strain, and Cocal strain of vesicular stomatitis virus (VSV).

According to the embodiments of the present disclosure, the N protein has an amino acid sequence as set forth in SEQ ID NO: 1, SEQ ID NO: 7, SEQ ID NO: 13, SEQ ID NO: 19, or SEQ ID NO: 25.

(SEQ ID NO: 1)
MSVTVKRIIDNTVVVPKLPANEDPVEYPADYFRKSKEIPLYINTTKSLS
DLRGYVYQGLKSGNVSIIHVNSYLYGALKDIRGKLDKDWSSFGINIGKA
GDTIGIFDLVSLKALDGVLPDGVSDASRTSADDKWLPLYLLGLYRVGRT
QMPEYRKKLMDGLTNQCKMINEQFEPLVPEGRDIFDVWGNDSNYTKIVA
AVDMFFHMFKKHECASFRYGTIVSRFKDCAALATFGHLCKITGMSTEDV
TTWILNREVADEMVQMMLPGQEIDKADSYMPYLIDFGLSSKSPYSSVKN
PAFHFWGQLTALLLRSTRARNARQPDDIEYTSLTTAGLLYAYAVGSSAD
LAQQFCVGDNKYTPDDSTGGLTTNAPPQGRDVVEWLGWFEDQNRKPTPD
MMQYAKRAVMSLQGLREKTIGKYAKSEFDK.
(SEQ ID NO: 7)
MSVTVKRIIDNTVIVPKLPANEDPVEYPADYFRKSKEIPLYINTTKSLS
DLRGYVYQGLKSGNVSIIHVNSYLYGALKDIRGKLDKDWSSFGINIGKA
GDTIGIFDLVSLKALDGVLPDGVSDASRTSADDKWLPLYLLGLYRVGRT
QMPEYRKKLMDGLTNQCKMINEQFEPLVPEGRDIFDVWGNDSNYTKIVA
AVDMFFHMFKKHECASFRYGTIVSRFKDCAALATFGHLCKITGMSTEDV
TTWILNREVADEMVQMMLPGQEIDKADSYMPYLIDFGLSSKSPYSSVKN
PAFHFWGQLTALLLRSTRARNARQPDDIEYTSLTTAGLLYAYAVGSSAD
LAQQFCVGDNKYTPDDSTGGLTTNAPPQGRDVVEWLGWFEDQNRKPTPD
MMQYAKRAVMSLQGLREKTIGKYAKSEFDK.
(SEQ ID NO: 13)
MAPTVKRIINDSIIQPKLPANEDPVEYPADYFKNNTNIVLYVSTKVALN
DLRAYVYQGIKSGNPSILHINAYLYAALKGVEGTLDRDWVSFGRTIGKR
EENVKIFDLVKVEELKTALPDGKSDPDRSAEDDKWLPIYILGLYRVGRS
KVTDYRKKLLDGLENQCRVASTRFESLVEDGLDFFDIWENDPNFTKIVA
AVDMFFHMFKKHERAPIRYGTIVSRFKDCAALATFGHLSKVSGLSIEDL
TTWVLNREVADELCQMMYPGQEIDKADSYMPYMIDFGLSQKSPYSSVKN
PAFHFWGQLAALLLRSTRAKNARQPDDIEYTSLTCASLLLSFAVGSSAD
IEQQFYIGEDKYTTEKDDSLKKSDVPPKGRNVVDWLGWYDDNGGKPTPD
MLNFARRAVSSLQSLREKTIGKYAKVEFDK.
(SEQ ID NO: 19)
MSSQVFCISTGQTVSVCLPANEDPVEFPGAFFTPNARKPTVYIKKETDL
SLLRSHVYDGIKDGSVTVSQINSYLYMVLKDIREKPDKNWTSFGVELGK
KNEPMGIFDLLNVEDVKGKELDKKGQDTRLPGDDLWLPTLILGLYRVSR
ATQVEYKKTLMTNLYAQCKLRTKDAEEIVDETAEFFNAWANDSNFTKIV
AAVDMYFHHFKKSDHAPIRFGTIVSRFKDCAALSTLSHLQKVTGLPIEE
VFTWVFNKSVQDDLLRMMTPGQEIDQADSYMPYLIDMGLSTKSPYSSTK
NPSFHFWGQLTAFLVKSARAKNALVPVDIAYHELTTAALLFAYAIGRSS
ELEQRFVLNGKKFTKEKDSRDDNDTTPPSERNVVVWLAWWEDIKHEITP
DMKAFAKRAVERVGDIRVNSVAEYARKLFA.
(SEQ ID NO: 25)
MDADKIVFKVNNQVVSLKPEIIVDQYEYKYPAIKDLKKPCITLGKAPDL
NKAYKSVLSGMSAAKLNPDDVCSYLAAAMQFFEGTCPEDWTSYGIVIAR
KGDKITPGSLVEIKRTDVEGNWALTGGMELTRDPTVPEHASLVGLLLSL
YRLSKISGQNTGNYKTNIADRIEQIFETAPFVKIVEHHTLMTTHKMCAN
WSTIPNFRFLAGTYDMFFSRIEHLYSAIRVGTVVTAYEDCSGLVSFTGF
IKQINLTAREAILYFFHKNFEEEIRRMFEPGQETAVPHSYFIHFRSLGL
SGKSPYSSNAVGHVFNLIHFVGCYMGQVRSLNATVIAACAPHEMSVLGG
YLGEEFFGKGTFERRFFRDEKELQEYEAAELTKTDVALADDGTVNSDDE
DYFSGETRSPEAVYTRIMMNGGRLKRSHIRRYVSVSSNHQARPNSFAEF
LNKTYSSDS.

According to the embodiments of the present disclosure, the P protein has an amino acid sequence as set forth in SEQ ID NO: 2, SEQ ID NO: 8, SEQ ID NO: 14, SEQ ID NO: 20, or SEQ ID NO: 26.

(SEQ ID NO: 2)
MDNLTKVREYLKSYSRLDQAVGEIDEIEAQRAEKSNYELFQEDGVEEHT
KPSYFQAADDSDTESEPEIEDNQGLYAPDPEAEQVEGFIQGPLDDYADE
EVDVVFTSDWKQPELESDEHGKTLRLTSPEGLSGEQKSQWLSTIKAVVQ
SAKYWNLAECTFEASGEGVIMKERQITPDVYKVTPVMNTHPSQSEAVSD
VWSLSKTSMTFQPKKASLQPLTISLDELFSSRGEFISVGGDGRMSHKEA
ILLGLRYKKLYNQARVKYSL.
(SEQ ID NO: 8)
MDNLTKVREYLKSYSRLDQAVGEIDEIEAQRAEKSNYELFQEDGVEEHT
KPSYFQAADDSDTESEPEIEDNQGLYAQDPEAEQVEGFIQGPLDDYADE
EVDVVFTSDWKPPELESDEHGKTLRLTSPEGLSGEQKSQWLSTIKAVVQ
SAKYWNLAECTFEASGEGVIMKERQITPDVYKVTPVMNTHPSQSEAVSD
VWSLSKTSMTFQPKKASLQPLTISLDELFSSRGEFISVGGDGRMSHKEA
ILLGLRYKKLYNQARVKYSL.
(SEQ ID NO: 14)
MDSVDRLKTYLATYDNLDSALQDANESEERREDKYLQDLFIEDQGDKPT
PSYYQEEESSDSDTDYNAEHLTMLSPDERIDKWEEDLPELEKIDDDIPV
TFSDWTQPVMKENGGEKSLSLFPPVGLTKIQTEQWKKTIEAVCESSKYW
NLSECQILNLEDSLTLKGRLMTPDCSSSVKSQNSVRRSEPLYSSHSPGP
PLKVSESINLWDLKSTEVQLISKRAGVKDMTVKLTDFFGSEEEYYSVCP
EGAPDLMGAIIMGLKYKKLFNQARMKYRL.
(SEQ ID NO: 20)
MEDSQLYQALKNYPKLQDTLDSIENLEDDTKSEPSECGSPTERGIPSYY
LAEELDECEEEDSEDDDDNLPTEIPDPPTVDMLEAIMEDEIDDTAYQVH
FEAKQTWKPVIETGGNERGKFTLSVPQNLSALQLLQWETGIHALAERLG
GCRLLQISTRGTRDGIEFTVRETPCVSPASDPIPSTSRSSSIASNVSTR
QTESPGSKSNTSLGIPEVPANLIDIGAIDKEFILAAISPSDPPYKNTLR
NLFGSGDSFEQYNQTGIYSLKELVVAGLKRKGIYNRIRIRCHLEPQFN.
(SEQ ID NO: 26)
MSKIFVNPSAIRAGLADLEMAEETVDLINRNIEDNQAHLQGEPIEVDNL
PEDMGRLHLDDGKSPNHGEIAKVGEGKYREDFQMDEGEDPSFLFQSYLE
NVGVQIVRQMRSGERFLKIWSQTVEEIISYVAVNFPNPPGKSSEDKSTQ
TTGRELKKETTPTPSQRESQSSKARMAAQIASGPPALEWSATNEEDDLS
VEAEIAHQIAESFSKKYKFPSRSSGILLYNFEQLKMNLDDIVKEAKNVP
GVTRLAHDGSKLPLRCVLGWVALANSKKFQLLVESDKLSKIMQDDLNRY
TSC.

According to the embodiments of the present disclosure, the L protein has an amino acid sequence as set forth in SEQ ID NO: 3, SEQ ID NO: 9, SEQ ID NO: 15, SEQ ID NO: 21, or SEQ ID NO: 27.

(SEQ ID NO: 3)
MEVHDFETDEFNDFNEDDYATREFLNPDERMTYLNHADYNLNSPLISDDID
NLIRKFNSLPIPSMWDSKNWDGVLEMLTSCQANPIPTSQMHKWMGSWLMSDNHDAS
QGYSFLHEVDKEAEITFDVVETFIRGWGNKPIEYIKKERWTDSFKILAYLCQKFLDLH
KLTLILNAVSEVELLNLARTFKGKVRRSSHGTNICRIRVPSLGPTFISEGWAYFKKLDIL
MDRNFLLMVKDVIIGRMQTVLSMVCRIDNLFSEQDIFSLLNIYRIGDKIVERQGNFSY
DLIKMVEPICNLKLMKLARESRPLVPQFPHFENHIKTSVDEGAKIDRGIRFLHDQIMSV
KTVDLTLVIYGSFRHWGHPFIDYYTGLEKLHSQVTMKKDIDVSYAKALASDLARIVLF
QQFNDHKKWFVNGDLLPHDHPFKSHVKENTWPTAAQVQDFGDKWHELPLIKCFEIP
DLLDPSIIYSDKSHSMNRSEVLKHVRMNPNTPIPSKKVLQTMLDTKATNWKEFLKEID
EKGLDDDDLIIGLKGKERELKLAGRFFSLMSWKLREYFVITEYLIKTHFVPMFKGLTM
ADDLTAVIKKMLDSSSGQGLKSYEAICIANHIDYEKWNNHQRKLSNGPVFRVMGQFL
GYPSLIERTHEFFEKSLIYYNGRPDLMRVHNNTLINSTSQRVCWQGQEGGLEGLRQKG
WSILNLLVIQREAKIRNTAVKVLAQGDNQVICTQYKTKKSRNVVELQGALNQMVSNN
EKIMTAIKIGTGKLGLLINDDETMQSADYLNYGKIPIFRGVIRGLETKRWSRVTCVIND
QIPTCANIMSSVSTNALTVAHFAENPINAMIQYNYFGTFARLLLMMHDPALRQSLYEV
QDKIPGLHSSTFKYAMLYLDPSIGGVSGMSLSRFLIRAFPDPVTESLSFWRFIHVHARS
EHLKEMSAVFGNPEIAKFRITHIDKLVEDPTSLNIAMGMSPANLLKTEVKKCLIESRQTI
RNQVIKDATIYLYHEEDRLRSFLWSINPLFPRFLSEFKSGTFLGVADGLISLFQNSRTIRN
SFKKKYHRELDDLIVRSEVSSLTHLGKLHLRRGSCKMWTCSATHADTLRYKSWGRTV
IGTTVPHPLEMLGPQHRKETPCAPCNTSGFNYVSVHCPDGIHDVFSSRGPLPAYLGSK
TSESTSILQPWERESKVPLIKRATRLRDAISWFVEPDSKLAMTILSNIHSLTGEEWTKRQ
HGFKRTGSALHRFSTSRMSHGGFASQSTAALTRLMATTDTMRDLGDQNFDFLFQATL
LYAQITTTVARDGWITSCTDHYHIACKSCLRPIEEITLDSSMDYTPPDVSHVLKTWRNG
EGSWGQEIKQIYPLEGNWKNLAPAEQSYQVGRCIGFLYGDLAYRKSTHAEDSSLFPLS
IQGRIRGRGFLKGLLDGLMRASCCQVIHRRSLAHLKRPANAVYGGLIYLIDKLSVSPPF
LSLTRSGPIRDELETIPHKIPTSYPTSNRDMGVIVRNYFKYQCRLIEKGKYRSHYSQLW
LFSDVLSIDFIGPFSISTTLLQILYKPFLSGKDKNELRELANLSSLLRSGEGWEDIHVKFF
TKDILLCPEEIRHACKFGIAKDNNKDMSYPPWGRESRGTITTIPVYYTTTPYPKMLEM
PPRIQNPLLSGIRLGQLPTGAHYKIRSILHGMGIHYRDFLSCGDGSGGMTAALLRENV
HSRGIFNSLLELSGSVMRGASPEPPSALETLGGDKSRCVNGETCWEYPSDLCDPRTWD
YFLRLKAGLGLQIDLIVMDMEVRDSSTSLKIETNVRNYVHRILDEQGVLIYKTYGTYI
CESEKNAVTILGPMFKTVDLVQTEFSSSQTSEVYMVCKGLKKLIDEPNPDWSSINESW
KNLYAFQSSEQEFARAKKVSTYFTLTGIPSQFIPDPFVNIETMLQIFGVPTGVSHAAALK
SSDRPADLLTISLFYMAIISYYNINHIRVGPIPPNPPSDGIAQNVGIAITGISFWLSLMEKD
IPLYQQCLAVIQQSFPIRWEAVSVKGGYKQKWSTRGDGLPKDTRISDSLAPIGNWIRSL
ELVRNQVRLNPFNEILFNQLCRTVDNHLKWSNLRRNTGMIEWINRRISKEDRSILMLK
SDLHEENSWRD.
(SEQ ID NO: 9)
MEVHDFETDEFNDFNEDDYATREFLNPDERMTYLNHADYNLNSPLISDDID
NLIRKFNSLPIPSMWDSKNWDGVLEMLTSCQANPISTSQMHKWMGSWLMSDNHDAS
QGYSFLHEVDKEAEITFDVVETFIRGWGNKPIEYIKKERWTDSFKILAYLCQKFLDLH
KLTLILNAVSEVELLNLARTFKGKVRRSSHGTNICRIRVPSLGPTFISEGWAYFKKLDIL
MDRNFLLMVKDVIIGRMQTVLSMVCRIDNLFSEQDIFSLLNIYRIGDKIVERQGNFSY
DLIKMVEPICNLKLMKLARESRPLVPQFPHFENHIKTSVDEGAKIDRGIRFLHDQIMSV
KTVDLTLVIYGSFRHWGHPFIDYYTGLEKLHSQVTMKKDIDVSYAKALASDLARIVLF
QQFNDHKKWFVNGDLLPHDHPFKSHVKENTWPTAAQVQDFGDKWHELPLIKCFEIP
DLLDPSIIYSDKSHSMNRSEVLKHVRMNPNTPIPSKKVLQTMLDTKATNWKEFLKEID
EKGLDDDDLIIGLKGKERELKLAGRFFSLMSWKLREYFVITEYLIKTHFVPMFKGLTM
ADDLTAVIKKMLDSSSGQGLKSYEAICIANHIDYEKWNNHQRKLSNGPVFRVMGQFL
GYPSLIERTHEFFEKSLIYYNGRPDLMRVHNNTLINSTSQRVCWQGQEGGLEGLRQKG
WTILNLLVIQREAKIRNTAVKVLAQGDNQVICTQYKTKKSRNVVELQGALNQMVSN
NEKIMTAIKIGTGKLGLLINDDETMQSADYLNYGKIPIFRGVIRGLETKRWSRVTCVTN
DQIPTCANIMSSVSTNALTVAHFAENPINAMIQYNYFGTFARLLLMMHDPALRQSLYE
VQDKIPGLHSSTFKYAMLYLDPSIGGVSGMSLSRFLIRAFPDPVTESLSFWRFIHVHAR
SEHLKEMSAVFGNPEIAKFRITHIDKLVEDPTSLNIAMGMSPANLLKTEVKKCLIESRQ
TIRNQVIKDATIYLYHEEDRLRSFLWSINPLFPRFLSEFKSGTFLGVADGLISLFQNSRTI
RNSFKKKYHRELDDLIVRSEVSSLTHLGKLHLRRGSCKMWTCSATHADTLRYKSWG
RTVIGTTVPHPLEMLGPQHRKETPCAPCNTSGFNYVSVHCPDGIHDVFSSRGPLPAYL
GSKTSESTSILQPWERESKVPLIKRATRLRDAISWFVEPDSKLAMTILSNIHSLTGEEWT
KRQHGFKRTGSALHRFSTSRMSHGGFASQSTAALTRLMATTDTMRDLGDQNFDFLFQ
ATLLYAQITTTVARDGWITSCTDHYHIACKSCLRPIEEITLDSSMDYTPPDVSHVLKTW
RNGEGSWGQEIKQIYPLEGNWKNLAPAEQSYQVGRCIGFLYGDLAYRKSTHAEDSSL
FPLSIQGRIRGRGFLKGLLDGLMRASCCQVIHRRSLAHLKRPANAVYGGLIYLIDKLSV
SPPFLSLTRSGPIRDELETIPHKIPTSYPTSNRDMGVIVRNYFKYQCRLIEKGKYRSHYS
QLWLFSDVLSIDFIGPFSISTTLLQILYKPFLSGKDKNELRELANLSSLLRSGEGWEDIH
VKFFTKDILLCPEEIRHACKFGIAKDNNKDMSYPPWGRESRGTITTIPVYYTTTPYPKM
LEMPPRIQNPLLSGIRLGQLPTGAHYKIRSILHGMGIHYRDFLSCGDGSGGMTAALLR
ENVHSRGIFNSLLELSGSVMRGASPEPPSALETLGGDKSRCVNGETCWEYPSDLCDPR
TWDYFLRLKAGLGLQIDLIVMDMEVRDSSTSLKIETNVRNYVHRILDEQGVLIYKTY
GTYICESEKNAVTILGPMFKTVDLVQTEFSSSQTSEVYMVCKGLKKLIDEPNPDWSSIN
ESWKNLYAFQSSEQEFARAKKVSTYFTLTGIPSQFIPDPFVNIETMLQIFGVPTGVSHAA
ALKSSDRPADLLTISLFYMAIISYYNINHIRVGPIPPNPPSDGIAQNVGIAITGISFWLSLM
EKDIPLYQQCLAVIQQSFPIRWEAVSVKGGYKQKWSTRGDGLPKDTRTSDSLAPIGNW
IRSLELVRNQVRLNPFNEILFNQLCRTVDNHLKWSNLRRNTGMIEWINRRISKEDRSIL
MLKSDLHEENSWRD.
(SEQ ID NO: 15)
MDFDLIEDSANWEDDESDFFLRDILSQEDQMSYLNTADYNLNSPLISDDM
VYIIKRMNHEEVPPIWRSKEWDSPLDMLRGCQAQPMSHQEMHNWFGTWIQNVQHD
SAQPFTFLKEVDKESEMTYDLVSTFLKGWVGKDYPFKSKNKEIDSMALVGPLCQKFL
DLHKITLILNAVSLGETKELLTTFKGKYRMSCENIPIARLRLPSLGPVFMCKGWTYIHK
ERVLMDRTSFSCVKDVIIGRMQTFLSMIGRSDNKFSPDQIYTLANVYRIGDKILEQCGN
KAYDLIKMIEPICNLKMMELARLHRPKIPKFPHFEEHVKGSVQELTQRSNRIQTLYDLI
MSMKDVDLVLVVYGSFRHWGHPFIDYFQGLEKLHTQVNMEKHIDKEYPQQLASDLA
RLVLNKQFSESKKWFVDPSKMSPKHPFYEHVINKTWPTAAKIQDFGDNWHKLPLIQC
FEIPDLIDPSVIYSDKSHSMNKKEVIQHVRSKPNIPIPSNKVLQTMLTNRATNWKAFLK
DIDENGLDDDDLIIGLKGKERELKIAGRFFSLMSWRLREYFVITEYLIKTYYVPLFKGL
TMADDLTSVIKKMMDSSSGQGLDDYSSVCLANHIDYEKWNNHQRKESNGPIFRVMG
QFLGYPSLIERTHEFFEKSLIYYNGRPDLMTIRNGTLCNSTKHRVCWNGQKGGLEGLR
QKGWSIVNLLVIQREAKIRNTAVKVLAQGDNQVICTQYKTKKTRSELELRAVLHQMA
GNNNKIMEEIKRGTEKLGLIINDDETMQSADYLNYGKIPIFRGVIRGLETKRWSRVTC
VTNDQIPTCANLMSSVSTNALTVAHFAENPINAMIQYNYFGTFARLLLFMHDPAIRQSL
YKVQEKIPGLHTRTFKYAMLYLDPSIGGVCGMALSRFLIRAFPDPVTESLSFWKFIYEH
ASEPHLKKMEVMFETPQGAKFRIEHINKLLEDPTSLNISMGMSPANLLKSEVKKCLIES
RSSIKNEIIKDATIYMHQEEEKLRGFLWSIKPLFPRFLSEFKAGTFLGVSEGFINLFQNSR
TIRNSFKKRYHKDLDELIIKREISSLSHLGSMHYRLGDNQIWSCSASRADILRYKSWTR
KVVGTTVPHPLEMHGPPSKKERPCQLCNSSGLTYISVHCPKGIIDVFNRRGPLPAYLGS
NTSESTSILQPWEKESKIPIIKRATRLRDAISWFIPPESPLSTCILNNIQALTGEDWSSKQH
GFKRTGSALHRFSTSRMSNGGFASQSPATLTRMIATTDTMRDFGTKNYDFMFQASLLY
GQMTTSISRYGTPGSCTDHYHIRCKGCIREIEEVELNTSLEYKTPDVSHILEKWRNNTG
SWGHQIKQLKPAEGNWESLSPVEQSYQVARCIGFLYGELTHKKSRQADDSSLFPLSIQ
LKVRGRGFLRGLLDGLMRSSCCQVIHRRSVSTLKRPANAVYGGLIYLIDKLSASSPFLS
LVRTGPIRQELEQVPHKMSTSYPTNIRDLGSIVRNYFKYQCRPVERGNYKTYYNQIWL
FSDVLSTEFIGPMRISSSLLRLLYRPSLTKKDREELRELAALSSNLRSGEDWDDLHIKFF
SNDLLFCSQEIRHACKFGIKKDNEDITFYPNWGTEYIGNVTDIPVFYRAQNVKKDIRVP
PRIQNPLMSGLRLGQLPTGAHYKMRAIVFRLKIPYHDFLDCGDGSGGMTAALLRYNR
TSRGIFNSLLDLSDTMLRGSSPEPPSALETLGGERVRCVNGDSCWEHPSDLSDENTWK
YFLHLKKGCGMSINLITMDMEVQDSVISYKIESLVRQYVPVLLESDGCLIYKTYGTYI
ATQEDNSLTLIGSLFHSVQLVQTDLSSSNTSELYLVCRRLKDYVDTPFVDWIELYDNW
EKQYAFRSFKDEFQRAQSLTPETTLIGIPPQFVPDPGVNLETLFQIAGVPTGVAHGITHH
ILQSKDKLISNAIGSMCVISHFHINTIRTTDSMPGPPSDGDVNKMCSALIGACFWLDW
MESDLNLYKHCLRSIMKSMPVRWFRTLKNEKWSQKWDCKGDAIPKDSRLGDSLANI
GNWIRAWELIRNGNKSEPFDSMVAEALTKSVDKSLSWRKISKSTGIPRLLNSDIDLVD
QSILNVQIDIVENQAWQN.
(SEQ ID NO: 21)
MDLNPVDDAAELSEENFFSGKLSKECRIRGLNSVDYNLNSPLVSDDLTYLL
NKFKGKPVPIRWKMKKWDSILDQLRKHDLEYLRPSDLHQWFAEWMLYSKHGSKQG
EDFLKTVDEEASDTFEVVRSFIRGWTGGEINFVRKSGKHMGYCAELCQKFLDLHKLT
LLGNAATDNELLQLSKTFGDDKIYKKRLIKLPSLGRVIFDSGFFIVLDQRVLMDRNFM
LMMKDVIIGRMQTVLSMISRCDDKFSSKDIDFLLKVYSTGDKIIRKLGNDGYELIKTV
EPMCNLRLSDLARRFRPLVPPFPHFRRHIESTVDELSAKTPLIRELFSLIDTSPNVDSTLV
VYGSFRHWGHPFINYFEGLEKLHKQVTMEKEIDTNYSEALASDLARIVLTKEFNEKK
QWAVDYHRVPTSHPFKNHIRDNTWPTAAVIQDFGDHWHELPLIQCFDIPDLIDPSIIYS
DKSHSMNRSEVLNHVRTKPHTPIPSKKVLESMIDKPATNWLEFLEEIDKNGLSDEDLVI
GLKGKERELKIAGRFFSLMSWKLREYFVVTEYLIKTHFVPLFHGLTMADDMTAVIKK
MLESSSGQGLTNYDSVCIANHIDYEKWNNHQRKLSNGPVFKVMGQFLGYPNLIYRTH
EFFEKSLIYYNERPDLMKVRNGILENSTHQRVCWNGQAGGLEGLRQKGWSILNLLVI
QREAKIRNTAVKVLAQGDNQVICTQYKTKQYRNDIELRQALNQMAANNDVIMKAIE
SGTNKLGLLINQDETMQSADYLNYGKVPIFRGVIRGLETKRWSRVTCVINDQLPTCA
NLMSSVSTNALTVAHFDVHPLNAMIQFNFFGNFARLLLIMHDPAIRQSLNQLKGPNIN
VHSYGFKVAMLYLDPSIGGVCGTALSRFLIRSFPDPVTESLSFWKLIHHSTSDIRLKNLS
EQFGNPKIAVFRESHIEKLLEDPTSLNISMGMSAANLLKTEIKKNLLQKKSSIGNQIVK
DAVYYIHSEDEKLRTFLWSITPLFPRFLSEFKAGTFMGVASSIVSLFQNSRTIRNVFRDY
MSQTIDDLIVKSELTSLEHLSNYTDRKGSGGIWSCSAEQADKLRRMSWKRPVLGTTV
PHPLEMHGRGTLKSPLSKCCKESRMDYISVHIPEGLNKVLDGRGSLPAYLGSKTSEST
SILQPWEKESKIPIIRRATRLRDAIHWFVDPDSNLARSILNNIESLTGEKWEGALKGYK
RTGSALHRFSTSRVSHGGFSSQSPACLTRMMATTDTMRDYAQLNYDFMFQASLLYSQ
MTSSVILMGTTVSNTIHFHVTCRKCIREITEPMLESPREYRGKDVHLVLAKWKNSSNG
WGETLQLLKPVEGDWDTIPPVEKSYHVGRILGFLYGDLKSQNSSRADDSSIFPLSIQM
RLRGRGFLRGILDGLVRASACQVIHRRSVALLSKPANAIYGGLIYLIDKISASTSFTTLC
RDGPIREELSSIPHKIPTSYPTSNSDMGLHIRNYLKFQCKTVELGKYQSDIKDLWLFSD
VMTSNIAGPFALSTKILKCLYKPALSQKDRNNIRKISNFSKMMRSQLSWDPTSSEFITS
QILVCNEEIRHACKFGIPKLSLKFDDPVWGPEDYGLIWSIPVDYSSQSVPKNLKPCPRI
QNPSISGFRLGQLPTGAHYKLRSILRKKNIHYRDALCGGDGSGGMTAAVLRYNLKAR
AIFNSILDFDGSTMKGASPDPPSALETVVNGRTRCVNAESCWENPSDLSEQRTWDYFK
FLKTHHGLKIDLIVLDMEVRDFAISASIEKCVRNNVSSILEEDGVLIYKTYGSTIAAESS
NAVVNIGVLFESVELIQTEYSSTSTSEVYMYCRKIKKFVDAPHPDWISLDYYWSKLFC
FRSYKEEFFRSYEVSRKESLKGIPNSFIPDPLVNLETLLQIAGVPSGISHQLAIDIKESQLT
QITAAMVLCGMIANYTLDVTKKRDSYNPPSDGRLIRMSAALVGISFWISVKYYDKEL
NFELEQILSRSFPIRWMLSRNYLFWTTKKGFRNAKDVRLSGNMANIGNWIRCMELLH
LPPGSLSKDEVTTTCGKYIRSLKYSVILQQTGIIDLWKSRVASDDRSLMEVKTEFIESEH
WVD.
(SEQ ID NO: 27)
MLDPGEVYDDPIDPIELEAEPRGTPIVPNILRNSDYNLNSPLIEDPARLMLE
WLKTGNRPYRMTLTDNCSRSFRVLKDYFKKVDLGSLKVGGMAAQSMISLWLYGAHS
ESNRSRRCITDLAHFYSKSSPIEKLLNLTLGNRGLRIPPEGVLSCLERVDYDNAFGRYL
ANTYSSYLFFHVITLYMNALDWDEEKTILALWKDLTSVDIGKDLVKFKDQIWGLLIVT
KDFVYSQSSNCLFDRNYTLMLKDLFLSRFNSLMVLLSPPEPRYSDDLISQLCQLYIAG
DQVLSMCGNSGYEVIKILEPYVVNSLVQRAEKFRPLIHSLGDFPVFIKDKVSQLEETFG
PCARRFFRALDQFDNIHDLVFVFGCYRHWGHPYIDYRKGLSKLYDQVHLKKVIDKSY
QECLASDLARRILRWGFDKYSKWYLDSRFLARDHPLTPYIKTQTWPPKHIVDLVGDT
WHKLPITQIFEIPESMDPSEILDDKSHSFTRTRLASWLSENRGGPVPSEKVIITALSKPPV
NPREFLRSIDLGGLPDEDLIIGLKPKERELKIEGRFFALMSWNLRLYFVITEKLLANYIL
PLFDALTMTDNLNKVFKKLIDRVTGQGLLDYSRVTYAFHLDYEKWNNHQRLESTED
VFSVLDQVFGLKRVFSRTHEFFQKAWIYYSDRSDLIGLREDQIYCLDASNGPTCWNGQ
DGGLEGLRQKGWSLVSLLMIDRESQIRNTRTKILAQGDNQVLCPTYMLSPGLSQEGLL
YELERISRNALSIYRAVEEGASKLGLIIKKEETMCSYDFLIYGKTPLFRGNILVPESKRW
ARVSCVSNDQIVNLANIMSTVSTNALTVAQHSQSLIKPMRDFLLMSVQAVFHYLLFSPI
LKGRVYKILSAEGESFLLAMSRIIYLDPSLGGISGMSLGRFHIRQFSDPVSEGLSFWREI
WLSSQESWIHALCQEAGNPDLGERTLESFTRLLEDPTTLNIRGGASPTILLKDAIRKAL
YDEVDKVENSEFREAILLSKTHRDNFILFLISVEPLFPRFLSELFSSSFLGIPESIIGLIQNS
RTIRRQFRKSLSKTLEESFYNSEIHGISRMTQTPQRVGGVWPCSSERADQLREISWGRK
VVGTTVPHPSEMLGLLPKSSISCTCGATGGGNPRVSVSVLPSFDQSFFSRGPLKGYLGS
STSMSTQLFHAWEKVTNVHVVKRALSLKESINWFITRDSNLAQALIRNIMSLTGPDFP
LEEAPVFKRTGSALHRFKSARYSEGGYSSVCPNLLSHISVSTDTMSDLTQDGKNYDFM
FQPLMLYAQTWTSELVQRDTRLRDSTFHWHLRCNRCVRPIDDVTLETSQIFEFPDVSK
RISRMVSGAVPHFQRLPDIRLRPGDFESLSGREKSHHIGSAQGLLYSILVAIHDSGYNDG
TIFPVNIYGKVSPRDYLRGLARGVLIGSSICFLTRMTNININRPLELVSGVISYILLRLDN
HPSLYIMLREPSLRGEIFSIPQKIPAAYPTTMKEGNRSILCYLQHVLRYEREIITASPEND
WLWIFSDFRSAKMTYLSLITYQSHLLLQRVERNLSKSMRDNLRQLSSLMRQVLGGHG
EDTLESDDNIQRLLKDSLRRTRWVDQEVRHAARTMTGDYSPNKKVSRKVGCSEWVC
SAQQVAVSTSANPAPVSELDIRALSKRFQNPLISGLRVVQWATGAHYKLKPILDDLNVF
PSLCLVVGDGSGGISRAVLNMFPDAKLVFNSLLEVNDLMASGTHPLPPSAIMRGGNDI
VSRVIDLDSIWEKPSDLRNLATWKYFQSVQKQVNMSYDLIICDAEVTDIASINRITLLM
SDFALSIDGPLYLVFKTYGTMLVNPNYKAIQHLSRAFPSVTGFITQVTSSFSSELYLRFS
KRGKFFRDAEYLTSSTLREMSLVLFNCSSPKSEMQRARSLNYQDLVRGFPEEIISNPYN
EMIITLIDSDVESFLVHKMVDDLELQRGTLSKVAIIIAIMIVFSNRVFNVSKPLTDPSFYP
PSDPKILRHFNICCSTMMYLSTALGDVPSFARLHDLYNRPITYYFRKQVIRGNVYLSW
SWSNDTSVFKRVACNSSLSLSSHWIRLIYKIVKTTRLVGSIKDLSREVERHLHRYNRWI
TLEDIRSRSSLLDYSCL.

According to the embodiments of the present disclosure, the Nsp protein has at least one of amino acid sequences as set forth in SEQ ID NO: 31 to SEQ ID NO: 34. The Applicant found that the core complex of the self-replicating RNA sequence derived from positive-strand RNA virus is nonstructural protein (Nsp).

(SEQ ID NO: 31)
MEKPVVNVDVDPQSPFVVQLQKSFPQFEVVAQQVTPNDHANARAFSHLA
SKLIELEVPTTATILDIGSAPARRMFSEHQYHCVCPMRSPEDPDRMMKYASKLAEKAC
KITNKNLHEKIKDLRTVLDTPDAETPSLCFHNDVTCNMRAEYSVMQDVYINAPGTIY
HQAMKGVRTLYWIGFDTTQFMFSAMAGSYPAYNTNWADEKVLEARNIGLCSTKLSE
GRTGKLSIMRKKELKPGSRVYFSVGSTLYPEHRASLQSWHLPSVFHLNGKQSYTCRC
DTVVSCEGYVVKKITISPGITGETVGYAVTHNSEGFLLCKVTDTVKGERVSFPVCTYIP
ATICDQMTGIMATDISPDDAQKLLVGLNQRIVINGRTNRNTNTMQNYLLPIIAQGFSK
WAKERKDDLDNEKMLGTRERKLTYGCLWAFRTKKVHSFYRPPGTQTCVKVPASFSA
FPMSSVWTTSLPMSLRQKLKLALQPKKEEKLLQVSEELVMEAKAAFEDAQEEARAE
KLREALPPLVADKGIEAAAEVVCEVEGLQADIGA.
(SEQ ID NO: 32)
ALVETPRGHVRIIPQANDRMIGQYIVVSPNSVLKNAKLAPAHPLADQVKIIT
HSGRSGRYAVEPYDAKVLMPAGGAVPWPEFLALSESATLVYNEREFVNRKLYHIAMH
GPAKNTEEEQYKVTKAELAETEYVFDVDKKRCVKKEEASGLVLSGELTNPPYHELAL
EGLKTRPAVPYKVETIGVIGTPGSGKSAIIKSTVTARDLVTSGKKENCREIEADVLRLR
GMQITSKTVDSVMLNGCHKAVEVLYVDEAFACHAGALLALIAIVRPRKKVVLCGDP
MQCGFFNMMQLKVHFNHPEKDICTKTFYKYISRRCTQPVTAIVSTLHYDGKMKTTNP
CKKNIEIDITGATKPKPGDIILTCFRGWVKQLQIDYPGHEVMTAAASQGLTRKGVYAV
ROKVNENPLYAITSEHVNVLLTRTEDRLVWKTLQGDPWIKQPTNIPKGNFQATIEDWE
AEHKGIIAAINSPTPRANPFSCKTNVCWAKALEPILATAGIVLTGCQWSELFPQFADDK
PHSAIYALDVICIKFFGMDLTSGLFSKQSIPLTYHPADSARPVAHWDNSPGTRKYGYDH
AIAAELSRRFPVFQLAGKGTQLDLQTGRTRVISAQHNLVPVNRNLPHALVPEYKEKQP
GPVKKFLNQFKHHSVLVVSEEKIEAPRKRIEWIAPIGIAGADKNYNLAFGFPPQARYD
LVFINIGTKYRNHHFQQCEDHAATLKTLSRSALNCLNPGGTLVVKSYGYADRNSEDV
VTALARKFVRVSAARPDCVSSNTEMYLIFRQLDNSRTRQFTPHHLNCVISSVYEGTRD
GVGA.
(SEQ ID NO: 33)
APSYRTKRENIADCQEEAVVNAANPLGRPGEGVCRAIYKRWPTSFTDSATE
TGTARMTVCLGKKVIHAVGPDFRKHPEAEALKLLQNAYHAVADLVNEHNIKSVAIPLL
STGIYAAGKDRLEVSLNCLTTALDRTDADVTIYCLDKKWKERIDAALQLKESVTELK
DEDMEIDDELVWIHPDSCLKGRKGFSTTKGKLYSYFEGTKFHQAAKDMAEIKVLFPN
DQESNEQLCAYILGETMEAIREKCPVDHNPSSSPPKTLPCLCMYAMTPERVHRLRSNN
VKEVTVCSSTPLPKHKIKNVQKVQCTKVVLFNPHTPAFVPARKYIEVPEQPTAPPAQA
EEAPEVVATPSPSTADNTSLDVTDISLDMDDSSEGSLFSSFSGSDNSITSMDSWSSGPSS
LEIVDRRQVVVADVHAVQEPAPIPPPRLKKMARLAAARKEPTPPASNSSESLHLSFGG
VSMSLGSIFDGETARQAAVQPLATGPTDVPMSFGSFSDGEIDELSRRVTESEPVLFGSFE
PGEVNSIISSRSAVSFPLRKQRRRRRSRRTEY.
(SEQ ID NO: 34)
MMPTEANKSRYQSRKVENQKAITTERLLSGLRLYNSATDQPECYKITYPKP
LYSSSVPANYSDPQFAVAVCNNYLHENYPTVASYQITDEYDAYLDMVDGTVACLDTAT
FCPAKLRSYPKKHEYRAPNIRSAVPSAMQNTLQNVLIAATKRNCNVTQMRELPTLDS
ATFNVECFRKYACNDEYWEEFARKPIRITTEFVTAYVARLKGPKAAALFAKTYNLVPL
QEVPMDRFVMDMKRDVKVTPGTKHTEERPKVQVIQAAEPLATAYLCGIHRELVRRLT
AVLLPNIHTLFDMSAEDFDAIIAEHFKQGDPVLETDIASFDKSQDDAMALTGLMILEDL
GVDQPLLDLIECAFGEISSTHLPTGTRFKFGAMMKSGMFLTLFVNTVLNVVIASRVLE
ERLKTSRCAAFIGDDNIIHGVVSDKEMAERCATWLNMEVKIIDAVIGERPPYFCGGFIL
QDSVTSTACRVADPLKRLFKLGKPLPADDEQDEDRRRALLDETKAWFRVGITGTLAVA
VTTRYEVDNITPVLLALRTFAQSKRAFQAIRGEIKHLYGGPK.

According to some specific embodiments of the present disclosure, the self-replicating core sequence includes one of the following six combinations of base sequences encoding the proteins:

the N protein has the amino acid sequence as set forth in SEQ ID NO: 1, the P protein has the amino acid sequence as set forth in SEQ ID NO: 2, and the L protein has the amino acid sequence as set forth in SEQ ID NO: 3,

the N protein has the amino acid sequence as set forth in SEQ ID NO: 7, the P protein has the amino acid sequence as set forth in SEQ ID NO: 8, and the L protein has the amino acid sequence as set forth in SEQ ID NO: 9,

the N protein has the amino acid sequence as set forth in SEQ ID NO: 13, the P protein has the amino acid sequence as set forth in SEQ ID NO: 14, and the L protein has the amino acid sequence as set forth in SEQ ID NO: 15,

the N protein has the amino acid sequence as set forth in SEQ ID NO: 19, the P protein has the amino acid sequence as set forth in SEQ ID NO: 20, and the L protein has the amino acid sequence as set forth in SEQ ID NO: 21,

the N protein has the amino acid sequence as set forth in SEQ ID NO: 25, the P protein has the amino acid sequence as set forth in SEQ ID NO: 26, and the L protein has the amino acid sequence as set forth in SEQ ID NO: 27,

the Nsp protein has the amino acid sequence as set forth in SEQ ID NO: 31 to SEQ ID NO: 34. The Applicant found that the core complexes of self-replicating RNA sequences derived from positive-strand RNA viruses are NSP1 (SEQ ID NO: 31), NSP2 (SEQ ID NO: 32), NSP3 (SEQ ID NO: 33) and NSP4 (SEQ ID NO: 34).

According to the embodiments of the present disclosure, the first RNA sequence in the self-replicating RNA molecule has a base sequence as set forth in SEQ ID NO: 4, SEQ ID NO: 10, SEQ ID NO: 16, SEQ ID NO: 22, or SEQ ID NO: 28.

(SEQ ID NO: 4)
AUGUCUGUUACAGUCAAGAGAAUCAUUGACAACACAGUCGUAGUUC
CAAAACUUCCUGCAAAUGAGGAUCCAGUGGAAUACCCGGCAGAUUACUUCAGA
AAAUCAAAGGAGAUUCCUCUUUACAUCAAUACUACAAAAAGUUUGUCAGAUCU
AAGAGGAUAUGUCUACCAAGGCCUCAAAUCCGGAAAUGUAUCAAUCAUACAUG
UCAACAGCUACUUGUAUGGAGCAUUAAAGGACAUCCGGGGUAAGUUGGAUAA
AGAUUGGUCAAGUUUCGGAAUAAACAUCGGGAAAGCAGGGGAUACAAUCGGA
AUAUUUGACCUUGUAUCCUUGAAAGCCCUGGACGGCGUACUUCCAGAUGGAGU
AUCGGAUGCUUCCAGAACCAGCGCAGAUGACAAAUGGUUGCCUUUGUAUCUAC
UUGGCUUAUACAGAGUGGGCAGAACACAAAUGCCUGAAUACAGAAAAAAGCUC
AUGGAUGGGCUGACAAAUCAAUGCAAAAUGAUCAAUGAACAGUUUGAACCUCU
UGUGCCAGAAGGUCGUGACAUUUUUGAUGUGUGGGGAAAUGACAGUAAUUAC
ACAAAAAUUGUCGCUGCAGUGGACAUGUUCUUCCACAUGUUCAAAAAACAUGA
AUGUGCCUCGUUCAGAUACGGAACUAUUGUUUCCAGAUUCAAAGAUUGUGCUG
CAUUGGCAACAUUUGGACACCUCUGCAAAAUAACCGGAAUGUCUACAGAAGAU
GUAACGACCUGGAUCUUGAACCGAGAAGUUGCAGAUGAAAUGGUCCAAAUGAU
GCUUCCAGGCCAAGAAAUUGACAAGGCCGAUUCAUACAUGCCUUAUUUGAUCG
ACUUUGGAUUGUCUUCUAAGUCUCCAUAUUCUUCCGUCAAAAACCCUGCCUUC
CACUUCUGGGGGCAAUUGACAGCUCUUCUGCUCAGAUCCACCAGAGCAAGGAA
UGCCCGACAGCCUGAUGACAUUGAGUAUACAUCUCUUACUACAGCAGGUUUGU
UGUACGCUUAUGCAGUAGGAUCCUCUGCCGACUUGGCACAACAGUUUUGUGUU
GGAGAUAACAAAUACACUCCAGAUGAUAGUACCGGAGGAUUGACGACUAAUGC
ACCGCCACAAGGCAGAGAUGUGGUCGAAUGGCUCGGAUGGUUUGAAGAUCAAA
ACAGAAAACCGACUCCUGAUAUGAUGCAGUAUGCGAAAAGAGCAGUCAUGUCA
CUGCAAGGCCUAAGAGAGAAGACAAUUGGCAAGUAUGCUAAGUCAGAAUUUG
ACAAAUGA.
(SEQ ID NO: 10)
AUGUCUGUUACAGUCAAGAGAAUCAUUGACAACACAGUCAUAGUUC
CAAAACUUCCUGCAAAUGAGGAUCCAGUGGAAUACCCGGCAGAUUACUUCAGA
AAAUCAAAGGAGAUUCCUCUUUACAUCAAUACUACAAAAAGUUUGUCAGAUCU
AAGAGGAUAUGUCUACCAAGGCCUCAAAUCCGGAAAUGUAUCAAUCAUACAUG
UCAACAGCUACUUGUAUGGAGCAUUAAAGGACAUCCGGGGUAAGUUGGAUAA
AGAUUGGUCAAGUUUCGGAAUAAACAUCGGGAAAGCAGGGGAUACAAUCGGA
AUAUUUGACCUUGUAUCCUUGAAAGCCCUGGACGGCGUACUUCCAGAUGGAGU
AUCGGAUGCUUCCAGAACCAGCGCAGAUGACAAAUGGUUGCCUUUGUAUCUAC
UUGGCUUAUACAGAGUGGGCAGAACACAAAUGCCUGAAUACAGAAAAAAGCUC
AUGGAUGGGCUGACAAAUCAAUGCAAAAUGAUCAAUGAACAGUUUGAACCUCU
UGUGCCAGAAGGUCGUGACAUUUUUGAUGUGUGGGGAAAUGACAGUAAUUAC
ACAAAAAUUGUCGCUGCAGUGGACAUGUUCUUCCACAUGUUCAAAAAACAUGA
AUGUGCCUCGUUCAGAUACGGAACUAUUGUUUCCAGAUUCAAAGAUUGUGCUG
CAUUGGCAACAUUUGGACACCUCUGCAAAAUAACCGGAAUGUCUACAGAAGAU
GUAACGACCUGGAUCUUGAACCGAGAAGUUGCAGAUGAAAUGGUCCAAAUGAU
GCUUCCAGGCCAAGAAAUUGACAAGGCCGAUUCAUACAUGCCUUAUUUGAUCG
ACUUUGGAUUGUCUUCUAAGUCUCCAUAUUCUUCCGUCAAAAACCCUGCCUUC
CACUUCUGGGGGCAAUUGACAGCUCUUCUGCUCAGAUCCACCAGAGCAAGGAA
UGCCCGACAGCCUGAUGACAUUGAGUAUACAUCUCUUACUACAGCAGGUUUGU
UGUACGCUUAUGCAGUAGGAUCCUCUGCCGACUUGGCACAACAGUUUUGUGUU
GGAGAUAACAAAUACACUCCAGAUGAUAGUACCGGAGGAUUGACGACUAAUGC
ACCGCCACAAGGCAGAGAUGUGGUCGAAUGGCUCGGAUGGUUUGAAGAUCAAA
ACAGAAAACCGACUCCUGAUAUGAUGCAGUAUGCGAAAAGAGCAGUCAUGUCA
CUGCAAGGCCUAAGAGAGAAGACAAUUGGCAAGUAUGCUAAGUCAGAAUUUG
ACAAAUGA.
(SEQ ID NO: 16)
AUGGCUCCUACAGUUAAGAGAAUCAUUAAUGACUCCAUAAUUCAGC
CUAAACUACCGGCCAAUGAGGAUCCGGUAGAGUAUCCAGCUGAUUACUUCAAA
AAUAACACCAACAUAGUAUUAUAUGUGAGCACUAAAGUAGCCCUAAAUGAUUU
AAGGGCCUAUGUCUACCAGGGAAUCAAAUCCGGUAAUCCAUCAAUACUCCACA
UAAAUGCUUAUCUUUAUGCCGCGUUAAAGGGAGUAGAAGGAACAUUAGACAG
AGACUGGGUUAGCUUUGGAAGAACGAUCGGAAAGAGGGAAGAGAAUGUCAAA
AUCUUCGAUCUAGUCAAAGUUGAGGAACUGAAGACAGCUCUUCCUGAUGGGAA
AUCGGAUCCAGAUCGAUCAGCUGAAGAUGAUAAAUGGCUUCCUAUUUACAUCC
UAGGCCUAUAUCGAGUGGGUAGAUCCAAAGUUACAGAUUACAGAAAGAAACUC
CUGGACGGUCUAGAAAAUCAGUGUAGAGUGGCAUCAACCAGAUUUGAAAGCCU
UGUUGAAGAUGGUCUUGACUUCUUUGACAUCUGGGAAAAUGAUCCCAACUUCA
CCAAAAUAGUUGCUGCAGUGGAUAUGUUUUUUCACAUGUUCAAGAAGCAUGA
ACGUGCUCCAAUCAGAUAUGGAACCAUAGUCUCACGAUUCAAAGACUGUGCAG
CACUUGCUACAUUUGGACAUCUCAGCAAGGUUAGCGGACUUUCAAUUGAAGAC
CUAACAACAUGGGUUCUGAAUCGAGAAGUCGCAGACGAACUAUGCCAAAUGAU
GUACCCAGGCCAAGAAAUUGACAAGGCAGAUUCAUACAUGCCAUAUAUGAUUG
AUUUCGGGUUAUCCCAAAAAUCUCCUUAUUCAUCAGUGAAGAAUCCAGCUUUC
CACUUCUGGGGUCAACUGGCCGCACUCUUGCUAAGAUCAACUCGAGCAAAAAA
UGCUCGGCAACCCGAUGACAUUGAGUACACUUCGCUGACUUGUGCAAGUUUAC
UACUGUCAUUUGCAGUUGGAUCUUCAGCAGACAUUGAACAACAAUUCUACAUC
GGAGAGGACAAAUACACAACAGAGAAAGAUGACAGUCUCAAGAAAUCUGAUG
UCCCACCGAAAGGAAGAAAUGUUGUAGACUGGCUCGGUUGGUAUGAUGACAAU
GGAGGGAAACCCACACCGGAUAUGCUCAACUUCGCACGAAGAGCAGUCAGUUC
GUUGCAGUCACUCCGCGAGAAAACAAUUGGCAAAUACGCAAAAGUAGAAUUUG
ACAAAUGA.
(SEQ ID NO: 22)
AUGAGUUCUCAAGUAUUCUGCAUUUCUACAGGUCAAACAGUCUCCG
UAUGCCUUCCAGCAAAUGAAGACCCAGUAGAGUUUCCAGGGGCGUUCUUCACU
CCAAAUGCUAGGAAACCGACGGUGUACAUAAAGAAGGAGACAGAUCUGAGUCU
UUUGAGGAGUCAUGUUUAUGACGGGAUUAAGGAUGGUUCUGUGACAGUGUCC
CAGAUAAAUUCUUAUCUAUACAUGGUUUUGAAAGAUAUCCGGGAGAAACCAG
AUAAAAAUUGGACUUCUUUCGGAGUCGAAUUGGGAAAAAAGAAUGAGCCAAU
GGGAAUUUUCGAUCUGUUGAAUGUUGAAGAUGUUAAGGGGAAAGAAUUGGAC
AAAAAAGGGCAAGACACCCGACUGCCCGGAGAUGAUCUAUGGCUUCCAACGCU
GAUCCUCGGUCUUUAUCGCGUGUCCAGGGCGACACAGGUCGAAUACAAGAAGA
CUCUGAUGACCAAUCUAUAUGCUCAGUGUAAAUUGCGCACGAAGGAUGCAGAG
GAGAUUGUGGAUGAAACAGCAGAAUUCUUCAAUGCUUGGGCAAAUGACUCCAA
CUUCACAAAAAUCGUAGCUGCUGUGGAUAUGUACUUCCACCACUUCAAGAAGA
GUGAUCAUGCUCCUAUUCGUUUCGGAACAAUUGUGUCUAGAUUCAAAGACUGU
GCAGCAUUGUCCACUCUGUCGCACCUCCAAAAGGUCACAGGGCUCCCCAUUGA
GGAGGUCUUCACCUGGGUUUUCAAUAAAUCAGUACAGGAUGAUUUGUUGCGG
AUGAUGACUCCUGGCCAGGAAAUUGACCAAGCUGAUUCCUAUAUGCCCUAUCU
GAUCGACAUGGGCUUGUCCACGAAGUCUCCUUACUCUUCAACAAAAAAUCCCA
GUUUCCAUUUCUGGGGGCAACUGACCGCAUUCUUGGUAAAAUCCGCAAGAGCA
AAGAAUGCGUUAGUCCCAGUUGACAUCGCAUACCAUGAACUGACCACUGCUGC
ACUGCUUUUUGCCUAUGCAAUUGGCAGAUCGUCUGAGCUGGAGCAGAGAUUCG
UGCUUAACGGGAAGAAGUUCACCAAGGAGAAGGACUCUCGAGAUGACAAUGAC
ACUACUCCGCCAUCGGAGAGAAAUGUUGUUGUUUGGCUGGCCUGGUGGGAGGA
CAUUAAGCAUGAAAUUACUCCGGACAUGAAGGCCUUUGCCAAGAGAGCAGUGG
AAAGGGUAGGAGAUAUUCGAGUGAACUCAGUUGCAGAGUAUGCCAGGAAACU
CUUUGCAUGA.
(SEQ ID NO: 28)
AUGGAUGCCGACAAGAUUGUAUUCAAAGUCAAUAAUCAGGUGGUCU
CUUUGAAGCCUGAGAUUAUCGUGGAUCAAUAUGAGUACAAGUACCCUGCCAUC
AAAGAUUUGAAAAAGCCCUGUAUAACCCUAGGAAAGGCUCCCGAUUUAAAUAA
AGCAUACAAGUCAGUUUUGUCAGGCAUGAGCGCCGCCAAACUUAAUCCUGACG
AUGUAUGUUCCUAUUUGGCAGCGGCAAUGCAGUUUUUUGAGGGGACAUGUCCG
GAAGACUGGACCAGCUAUGGAAUUGUGAUUGCACGAAAAGGAGAUAAGAUCA
CCCCAGGUUCUCUGGUGGAGAUAAAACGUACUGAUGUAGAAGGGAAUUGGGCU
CUGACAGGAGGCAUGGAACUGACAAGAGACCCCACUGUCCCUGAGCAUGCGUC
CUUAGUCGGUCUUCUCUUGAGUCUGUAUAGGUUGAGCAAAAUAUCCGGGCAAA
ACACUGGUAACUAUAAGACAAACAUUGCAGACAGGAUAGAGCAGAUUUUUGA
GACAGCCCCUUUUGUUAAAAUCGUGGAACACCAUACUCUAAUGACAACUCACA
AAAUGUGUGCUAAUUGGAGUACUAUACCAAACUUCAGAUUUUUGGCCGGAACC
UAUGACAUGUUUUUCUCCCGGAUUGAGCAUCUAUAUUCAGCAAUCAGAGUGGG
CACAGUUGUCACUGCUUAUGAAGACUGUUCAGGACUGGUAUCAUUUACUGGGU
UCAUAAAACAAAUCAAUCUCACCGCUAGAGAGGCAAUACUAUAUUUCUUCCAC
AAGAACUUUGAGGAAGAGAUAAGAAGAAUGUUUGAGCCAGGGCAGGAGACAG
CUGUUCCUCACUCUUAUUUCAUCCACUUCCGUUCACUAGGCUUGAGUGGGAAA
UCUCCUUAUUCAUCAAAUGCUGUUGGUCACGUGUUCAAUCUCAUUCACUUUGU
AGGAUGCUAUAUGGGUCAAGUCAGAUCCCUAAAUGCAACGGUUAUUGCUGCAU
GUGCUCCUCAUGAAAUGUCUGUUCUAGGGGGCUAUCUGGGAGAGGAAUUCUUC
GGGAAAGGGACAUUUGAAAGAAGAUUCUUCAGAGAUGAGAAAGAACUUCAAG
AAUACGAGGCGGCUGAACUGACAAAGACUGACGUAGCACUGGCAGAUGAUGGA
ACUGUCAACUCUGACGACGAGGACUACUUUUCAGGUGAAACCAGAAGUCCGGA
GGCUGUUUAUACUCGAAUCAUGAUGAAUGGAGGUCGACUAAAGAGAUCUCACA
UACGGAGAUAUGUCUCAGUCAGUUCCAAUCAUCAAGCCCGUCCAAACUCAUUC
GCCGAGUUUCUAAACAAGACAUAUUCGAGUGACUCAUAA.

According to the embodiments of the present disclosure, the second RNA sequence has a base sequence as set forth in SEQ ID NO: 5, SEQ ID NO: 11, SEQ ID NO: 17, SEQ ID NO: 23, or SEQ ID NO: 29.

(SEQ ID NO: 5)
AUGGAUAAUCUCACAAAAGUUCGUGAGUAUCUCAAGUCCUAUUCUC
GUCUGGAUCAGGCGGUAGGAGAGAUAGAUGAGAUCGAAGCACAACGAGCUGA
AAAGUCCAAUUAUGAGUUGUUCCAAGAGGAUGGAGUGGAAGAGCAUACUAAG
CCCUCUUAUUUUCAGGCAGCAGAUGAUUCUGACACAGAAUCUGAACCAGAAAU
UGAAGACAAUCAAGGCUUGUAUGCACCAGAUCCAGAAGCUGAGCAAGUUGAAG
GCUUUAUACAGGGGCCUUUAGAUGACUAUGCAGAUGAGGAAGUGGAUGUUGU
AUUUACUUCGGACUGGAAACAGCCUGAGCUUGAAUCUGACGAGCAUGGAAAGA
CCUUACGGUUGACAUCGCCAGAGGGUUUAAGUGGAGAGCAGAAAUCCCAGUGG
CUUUCGACGAUUAAAGCAGUCGUGCAAAGUGCCAAAUACUGGAAUCUGGCAGA
GUGCACAUUUGAAGCAUCGGGAGAAGGGGUCAUUAUGAAGGAGCGCCAGAUA
ACUCCGGAUGUAUAUAAGGUCACUCCAGUGAUGAACACACAUCCGUCCCAAUC
AGAAGCAGUAUCAGAUGUUUGGUCUCUCUCAAAGACAUCCAUGACUUUCCAAC
CCAAGAAAGCAAGUCUUCAGCCUCUCACCAUAUCCUUGGAUGAAUUGUUCUCA
UCUAGAGGAGAGUUCAUCUCUGUCGGAGGUGACGGACGAAUGUCUCAUAAAGA
GGCCAUCCUGCUCGGCCUGAGAUACAAAAAGUUGUACAAUCAGGCGAGAGUCA
AAUAUUCUCUGUAG.
(SEQ ID NO: 11)
AUGGAUAAUCUCACAAAAGUUCGUGAGUAUCUCAAGUCCUAUUCUC
GUCUGGAUCAGGCGGUAGGAGAGAUAGAUGAGAUCGAAGCACAACGAGCUGA
AAAGUCCAAUUAUGAGUUGUUCCAAGAGGAUGGAGUGGAAGAGCAUACUAAG
CCCUCUUAUUUUCAGGCAGCAGAUGAUUCUGACACAGAAUCUGAACCAGAAAU
UGAAGACAAUCAAGGUUUGUAUGCACAGGAUCCAGAAGCUGAGCAAGUUGAA
GGCUUUAUACAGGGGCCUUUAGAUGACUAUGCAGAUGAGGAAGUGGAUGUUG
UAUUUACUUCGGACUGGAAACCACCUGAGCUUGAAUCUGACGAGCAUGGAAAG
ACCUUACGGUUGACAUCGCCAGAGGGUUUAAGUGGAGAGCAGAAAUCCCAGUG
GCUUUCGACGAUUAAAGCAGUCGUGCAAAGUGCCAAAUACUGGAAUCUGGCAG
AGUGCACAUUUGAAGCAUCGGGAGAAGGGGUCAUUAUGAAGGAGCGCCAGAU
AACUCCGGAUGUAUAUAAGGUCACUCCAGUGAUGAACACACAUCCGUCCCAAU
CAGAAGCAGUAUCAGAUGUUUGGUCUCUCUCAAAGACAUCCAUGACUUUCCAA
CCCAAGAAAGCAAGUCUUCAGCCUCUCACCAUAUCCUUGGAUGAAUUGUUCUC
AUCUAGAGGAGAGUUCAUCUCUGUCGGAGGUGACGGACGAAUGUCUCAUAAAG
AGGCCAUCCUGCUCGGCCUGAGAUACAAAAAGUUGUACAAUCAGGCGAGAGUC
AAAUAUUCUCUGUAG.
(SEQ ID NO: 17)
AUGGACAGUGUUGAUAGGCUCAAGACUUACUUAGCCACUUAUGAUA
AUUUGGAUUCUGCCUUGCAGGAUGCCAAUGAAUCUGAGGAAAGACGAGAGGA
UAAAUAUCUCCAAGACCUCUUCAUCGAAGAUCAAGGAGAUAAACCAACUCCGU
CAUAUUAUCAGGAAGAAGAAUCGUCAGAUUCAGAUACUGAUUAUAAUGCUGA
ACAUCUUACGAUGCUGUCACCGGAUGAAAGAAUAGACAAGUGGGAAGAAGAU
UUGCCUGAAUUAGAAAAGAUUGAUGAUGAUAUACCGGUGACCUUUUCUGAUU
GGACACAGCCUGUAAUGAAGGAAAAUGGGGGAGAGAAAUCAUUGUCUCUGUU
CCCUCCAGUCGGGUUAACAAAGAUUCAAACAGAACAAUGGAAAAAAACCAUUG
AGGCGGUUUGUGAGAGUUCAAAAUAUUGGAAUUUAUCAGAAUGCCAAAUUCU
UAACUUGGAAGACAGCCUCACUCUCAAAGGCCGAUUGAUGACUCCUGAUUGUA
GUUCUUCAGUAAAAUCUCAAAAUUCUGUCCGGAGGUCAGAACCUCUCUACUCC
UCUCAUUCUCCAGGUCCCCCACUCAAGGUAUCAGAGUCCAUCAAUUUAUGGGA
UUUAAAGUCCACUGAAGUACAAUUGAUCUCCAAGAGAGCCGGAGUUAAGGACA
UGACAGUCAAAUUGACAGACUUCUUUGGAAGUGAGGAAGAGUAUUAUUCAGU
AUGCCCAGAAGGGGCGCCAGACUUGAUGGGAGCUAUCAUCAUGGGACUGAAGU
ACAAGAAACUCUUCAAUCAGGCAAGAAUGAAAUAUCGUCUUUAA.
(SEQ ID NO: 23)
AUGGAAGACUCUCAACUAUAUCAAGCUCUCAAGAAUUACCCAAAGCU
UCAGGACACUCUCGACUCAAUUGAGAAUCUUGAAGAUGACACAAAGUCAGAGC
CAUCUGAGUGUGGUUCCCCCACAGAGAGAGGAAUUCCCAGCUAUUACCUGGCU
GAAGAAUUGGAUGAAUGUGAGGAAGAAGAUUCGGAAGAUGAUGAUGACAAUU
UACCUACUGAGAUACCGGAUCCUCCUACUGUUGACAUGCUGGAGGCAAUUAUG
GAAGAUGAGAUUGAUGAUACGGCUUACCAGGUACAUUUUGAGGCCAAACAGAC
UUGGAAACCGGUGAUAGAGACAGGGGGAAAUGAAAGAGGUAAGUUCACCCUU
UCAGUCCCUCAGAAUCUGAGCGCUCUACAACUUCUCCAGUGGGAGACAGGGAU
UCAUGCAUUGGCGGAGAGACUUGGGGGAUGCAGACUGCUGCAGAUCAGCACAC
GGGGAACGAGGGAUGGGAUAGAGUUCACUGUAAGAGAAACGCCUUGCGUUUC
ACCUGCUUCUGAUCCAAUCCCUUCCACAUCCAGAUCCAGUUCAAUUGCUUCCA
AUGUCUCUACCAGACAAACCGAAUCACCUGGCUCCAAAUCCAAUACAAGUCUC
GGGAUACCAGAAGUCCCGGCUAAUUUGAUCGACAUAGGAGCAAUUGAUAAAGA
AUUCAUCCUGGCAGCUAUCUCUCCGUCUGACCCACCUUAUAAGAACACUCUGA
GAAACCUCUUCGGGUCCGGAGAUUCCUUUGAACAGUACAACCAGACAGGAAUU
UACUCUCUUAAGGAACUCGUCGUUGCUGGAUUGAAGAGAAAAGGAAUUUAUA
AUAGAAUCAGAAUCAGAUGUCAUCUUGAGCCCCAGUUCAAUUGA.
(SEQ ID NO: 29)
AUGAGCAAGAUCUUUGUCAAUCCUAGUGCUAUUAGAGCCGGUCUGG
CCGAUCUUGAGAUGGCUGAAGAAACUGUUGAUCUGAUCAAUAGAAAUAUCGA
AGACAAUCAGGCUCAUCUCCAAGGGGAACCCAUAGAGGUGGACAAUCUCCCUG
AGGAUAUGGGGCGACUUCACCUGGAUGAUGGAAAAUCGCCCAACCAUGGUGAG
AUAGCCAAGGUGGGAGAAGGCAAGUAUCGAGAGGACUUUCAGAUGGAUGAAG
GAGAGGAUCCUAGCUUCCUGUUCCAGUCAUACCUGGAAAAUGUUGGAGUCCAA
AUAGUCAGACAAAUGAGGUCAGGAGAGAGAUUUCUCAAGAUAUGGUCACAGA
CCGUAGAAGAGAUUAUAUCCUAUGUCGCGGUCAACUUUCCCAACCCUCCAGGA
AAGUCUUCAGAGGAUAAAUCAACCCAGACUACUGGCCGAGAGCUCAAGAAGGA
GACAACACCCACUCCUUCUCAGAGAGAAAGCCAAUCAUCGAAAGCCAGGAUGG
CGGCUCAAAUUGCUUCUGGCCCUCCAGCCCUUGAAUGGUCGGCUACCAAUGAA
GAGGAUGAUCUAUCAGUGGAGGCUGAGAUCGCUCACCAGAUUGCAGAAAGUUU
CUCCAAAAAAUAUAAGUUUCCCUCUCGAUCCUCAGGGAUACUCUUGUAUAAUU
UUGAGCAAUUGAAAAUGAACCUUGAUGAUAUAGUUAAAGAGGCAAAAAAUGU
ACCAGGUGUGACCCGUUUAGCCCAUGACGGGUCCAAACUCCCCCUAAGAUGUG
UACUGGGAUGGGUCGCUUUGGCCAACUCUAAGAAAUUCCAGUUGUUAGUCGAA
UCCGACAAGCUGAGUAAAAUCAUGCAAGAUGACUUGAAUCGCUAUACAUCUUG
CUAA.

According to the embodiments of the present disclosure, the third RNA sequence has a base sequence as set forth in SEQ ID NO: 6, SEQ ID NO: 12, SEQ ID NO: 18, SEQ ID NO: 24 or SEQ ID NO: 30.

(SEQ ID NO: 6)
AUGGAAGUCCACGAUUUUGAGACCGACGAGUUCAAUGAUUUCAAUG
AAGAUGACUAUGCCACAAGAGAAUUCCUGAAUCCCGAUGAGCGCAUGACGUAC
UUGAAUCAUGCUGAUUACAACCUGAAUUCUCCUCUAAUUAGUGAUGAUAUUGA
CAAUUUAAUCAGGAAAUUCAAUUCUCUUCCAAUUCCCUCGAUGUGGGAUAGUA
AGAACUGGGAUGGAGUUCUUGAGAUGUUAACGUCAUGUCAAGCCAAUCCCAUC
CCAACAUCUCAGAUGCAUAAAUGGAUGGGAAGUUGGUUAAUGUCUGAUAAUC
AUGAUGCCAGUCAAGGGUAUAGUUUUUUACAUGAAGUGGACAAAGAGGCAGA
AAUAACAUUUGACGUGGUGGAGACCUUCAUCCGCGGCUGGGGCAACAAACCAA
UUGAAUACAUCAAAAAGGAAAGAUGGACUGACUCAUUCAAAAUUCUCGCUUAU
UUGUGUCAAAAGUUUUUGGACUUACACAAGUUGACAUUAAUCUUAAAUGCUG
UCUCUGAGGUGGAAUUGCUCAACUUGGCGAGGACUUUCAAAGGCAAAGUCAGA
AGAAGUUCUCAUGGAACGAACAUAUGCAGGAUUAGGGUUCCCAGCUUGGGUCC
UACUUUUAUUUCAGAAGGAUGGGCUUACUUCAAGAAACUUGAUAUUCUAAUG
GACCGAAACUUUCUGUUAAUGGUCAAAGAUGUGAUUAUAGGGAGGAUGCAAA
CGGUGCUAUCCAUGGUAUGUAGAAUAGACAACCUGUUCUCAGAGCAAGACAUC
UUCUCCCUUCUAAAUAUCUACAGAAUUGGAGAUAAAAUUGUGGAGAGGCAGG
GAAAUUUUUCUUAUGACUUGAUUAAAAUGGUGGAACCGAUAUGCAACUUGAA
GCUGAUGAAAUUAGCAAGAGAAUCAAGGCCUUUAGUCCCACAAUUCCCUCAUU
UUGAAAAUCAUAUCAAGACUUCUGUUGAUGAAGGGGCAAAAAUUGACCGAGG
UAUAAGAUUCCUCCAUGAUCAGAUAAUGAGUGUGAAAACAGUGGAUCUCACAC
UGGUGAUUUAUGGAUCGUUCAGACAUUGGGGUCAUCCUUUUAUAGAUUAUUA
CACUGGACUAGAAAAAUUACAUUCCCAAGUAACCAUGAAGAAAGAUAUUGAUG
UGUCAUAUGCAAAAGCACUUGCAAGUGAUUUAGCUCGGAUUGUUCUAUUUCAA
CAGUUCAAUGAUCAUAAAAAGUGGUUCGUGAAUGGAGACUUGCUCCCUCAUGA
UCAUCCCUUUAAAAGUCAUGUUAAAGAAAAUACAUGGCCCACAGCUGCUCAAG
UUCAAGAUUUUGGAGAUAAAUGGCAUGAACUUCCGCUGAUUAAAUGUUUUGA
AAUACCCGACUUACUAGACCCAUCGAUAAUAUACUCUGACAAAAGUCAUUCAA
UGAAUAGGUCAGAGGUGUUGAAACAUGUCCGAAUGAAUCCGAACACUCCUAUC
CCUAGUAAAAAGGUGUUGCAGACUAUGUUGGACACAAAGGCUACCAAUUGGAA
AGAAUUUCUUAAAGAGAUUGAUGAGAAGGGCUUAGAUGAUGAUGAUCUAAUU
AUUGGUCUUAAAGGAAAGGAGAGGGAACUGAAGUUGGCAGGUAGAUUUUUCU
CCCUAAUGUCUUGGAAAUUGCGAGAAUACUUUGUAAUUACCGAAUAUUUGAU
AAAGACUCAUUUCGUCCCUAUGUUUAAAGGCCUGACAAUGGCGGACGAUCUAA
CUGCAGUCAUUAAAAAGAUGUUAGAUUCCUCAUCCGGCCAAGGAUUGAAGUCA
UAUGAGGCAAUUUGCAUAGCCAAUCACAUUGAUUACGAAAAAUGGAAUAACCA
CCAAAGGAAGUUAUCAAACGGCCCAGUGUUCCGAGUUAUGGGCCAGUUCUUAG
GUUAUCCAUCCUUAAUCGAGAGAACUCAUGAAUUUUUUGAGAAAAGUCUUAU
AUACUACAAUGGAAGACCAGACUUGAUGCGUGUUCACAACAACACACUGAUCA
AUUCAACCUCCCAACGAGUUUGUUGGCAAGGACAAGAGGGUGGACUGGAAGGU
CUACGGCAAAAAGGAUGGAGUAUCCUCAAUCUACUGGUUAUUCAAAGAGAGGC
UAAAAUCAGAAACACUGCUGUCAAAGUCUUGGCACAAGGUGAUAAUCAAGUUA
UUUGCACACAGUAUAAAACGAAGAAAUCGAGAAACGUUGUAGAAUUACAGGG
UGCUCUCAAUCAAAUGGUUUCUAAUAAUGAGAAAAUUAUGACUGCAAUCAAA
AUAGGGACAGGGAAGUUAGGACUUUUGAUAAAUGACGAUGAGACUAUGCAAU
CUGCAGAUUACUUGAAUUAUGGAAAAAUACCGAUUUUCCGUGGAGUGAUUAG
AGGGUUAGAGACCAAGAGAUGGUCACGAGUGACUUGUGUCACCAAUGACCAAA
UACCCACUUGUGCUAAUAUAAUGAGCUCAGUUUCCACAAAUGCUCUCACCGUA
GCUCAUUUUGCUGAGAACCCAAUCAAUGCCAUGAUACAGUACAAUUAUUUUGG
GACAUUUGCUAGACUCUUGUUGAUGAUGCAUGAUCCUGCUCUUCGUCAAUCAU
UGUAUGAAGUUCAAGAUAAGAUACCGGGCUUGCACAGUUCUACUUUCAAAUAC
GCCAUGUUGUAUUUGGACCCUUCCAUUGGAGGAGUGUCGGGCAUGUCUUUGUC
CAGGUUUUUGAUUAGAGCCUUCCCAGAUCCCGUAACAGAAAGUCUCUCAUUCU
GGAGAUUCAUCCAUGUACAUGCUCGAAGUGAGCAUCUGAAGGAGAUGAGUGCA
GUAUUUGGAAACCCCGAGAUAGCCAAGUUUCGAAUAACUCACAUAGACAAGCU
AGUAGAAGAUCCAACCUCUCUGAACAUCGCUAUGGGAAUGAGUCCAGCGAACU
UGUUAAAGACUGAGGUUAAAAAAUGCUUAAUCGAAUCAAGACAAACCAUCAG
GAACCAGGUGAUUAAGGAUGCAACCAUAUAUUUGUAUCAUGAAGAGGAUCGG
CUCAGAAGUUUCUUAUGGUCAAUAAAUCCUCUGUUCCCUAGAUUUUUAAGUGA
AUUCAAAUCAGGCACUUUUUUGGGAGUCGCAGACGGGCUCAUCAGUCUAUUUC
AAAAUUCUCGUACUAUUCGGAACUCCUUUAAGAAAAAGUAUCAUAGGGAAUU
GGAUGAUUUGAUUGUGAGGAGUGAGGUAUCCUCUUUGACACAUUUAGGGAAA
CUUCAUUUGAGAAGGGGAUCAUGUAAAAUGUGGACAUGUUCAGCUACUCAUGC
UGACACAUUAAGAUACAAAUCCUGGGGCCGUACAGUUAUUGGGACAACUGUAC
CCCAUCCAUUAGAAAUGUUGGGUCCACAACAUCGAAAAGAGACUCCUUGUGCA
CCAUGUAACACAUCAGGGUUCAAUUAUGUUUCUGUGCAUUGUCCAGACGGGAU
CCAUGACGUCUUUAGUUCACGGGGACCAUUGCCUGCUUAUCUAGGGUCUAAAA
CAUCUGAAUCUACAUCUAUUUUGCAGCCUUGGGAAAGGGAAAGCAAAGUCCCA
CUGAUUAAAAGAGCUACACGUCUUAGAGAUGCUAUCUCUUGGUUUGUUGAACC
CGACUCUAAACUAGCAAUGACUAUACUUUCUAACAUCCACUCUUUAACAGGCG
AAGAAUGGACCAAAAGGCAGCAUGGGUUCAAAAGAACAGGGUCUGCCCUUCAU
AGGUUUUCGACAUCUCGGAUGAGCCAUGGUGGGUUCGCAUCUCAGAGCACUGC
AGCAUUGACCAGGUUGAUGGCAACUACAGACACCAUGAGGGAUCUGGGAGAUC
AGAAUUUCGACUUUUUAUUCCAAGCAACGUUGCUCUAUGCUCAAAUUACCACC
ACUGUUGCAAGAGACGGAUGGAUCACCAGUUGUACAGAUCAUUAUCAUAUUGC
CUGUAAGUCCUGUUUGAGACCCAUAGAAGAGAUCACCCUGGACUCAAGUAUGG
ACUACACGCCCCCAGAUGUAUCCCAUGUGCUGAAGACAUGGAGGAAUGGGGAA
GGUUCGUGGGGACAAGAGAUAAAACAGAUCUAUCCUUUAGAAGGGAAUUGGA
AGAAUUUAGCACCUGCUGAGCAAUCCUAUCAAGUCGGCAGAUGUAUAGGUUUU
CUAUAUGGAGACUUGGCGUAUAGAAAAUCUACUCAUGCCGAGGACAGUUCUCU
AUUUCCUCUAUCUAUACAAGGUCGUAUUAGAGGUCGAGGUUUCUUAAAAGGG
UUGCUAGACGGAUUAAUGAGAGCAAGUUGCUGCCAAGUAAUACACCGGAGAAG
UCUGGCUCAUUUGAAGAGGCCGGCCAACGCAGUGUACGGAGGUUUGAUUUACU
UGAUUGAUAAAUUGAGUGUAUCACCUCCAUUCCUUUCUCUUACUAGAUCAGGA
CCUAUUAGAGACGAAUUAGAAACGAUUCCCCACAAGAUCCCAACCUCCUAUCC
GACAAGCAACCGUGAUAUGGGGGUGAUUGUCAGAAAUUACUUCAAAUACCAAU
GCCGUCUAAUUGAAAAGGGAAAAUACAGAUCACAUUAUUCACAAUUAUGGUU
AUUCUCAGAUGUCUUAUCCAUAGACUUCAUUGGACCAUUCUCUAUUUCCACCA
CCCUCUUGCAAAUCCUAUACAAGCCAUUUUUAUCUGGGAAAGAUAAGAAUGAG
UUGAGAGAGCUGGCAAAUCUUUCUUCAUUGCUAAGAUCAGGAGAGGGGUGGG
AAGACAUACAUGUGAAAUUCUUCACCAAGGACAUAUUAUUGUGUCCAGAGGAA
AUCAGACAUGCUUGCAAGUUCGGGAUUGCUAAGGAUAAUAAUAAAGACAUGA
GCUAUCCCCCUUGGGGAAGGGAAUCCAGAGGGACAAUUACAACAAUCCCUGUU
UAUUAUACGACCACCCCUUACCCAAAGAUGCUAGAGAUGCCUCCAAGAAUCCA
AAAUCCCCUGCUGUCCGGAAUCAGGUUGGGCCAAUUACCAACUGGCGCUCAUU
AUAAAAUUCGGAGUAUAUUACAUGGAAUGGGAAUCCAUUACAGGGACUUCUU
GAGUUGUGGAGACGGCUCCGGAGGGAUGACUGCUGCAUUACUACGAGAAAAUG
UGCAUAGCAGAGGAAUAUUCAAUAGUCUGUUAGAAUUAUCAGGGUCAGUCAU
GCGAGGCGCCUCUCCUGAGCCCCCCAGUGCCCUAGAAACUUUAGGAGGAGAUA
AAUCGAGAUGUGUAAAUGGUGAAACAUGUUGGGAAUAUCCAUCUGACUUAUG
UGACCCAAGGACUUGGGACUAUUUCCUCCGACUCAAAGCAGGCUUGGGGCUUC
AAAUUGAUUUAAUUGUAAUGGAUAUGGAAGUUCGGGAUUCUUCUACUAGCCU
GAAAAUUGAGACGAAUGUUAGAAAUUAUGUGCACCGGAUUUUGGAUGAGCAA
GGAGUUUUAAUCUACAAGACUUAUGGAACAUAUAUUUGUGAGAGCGAAAAGA
AUGCAGUAACAAUCCUUGGUCCCAUGUUCAAGACGGUCGACUUAGUUCAAACA
GAAUUUAGUAGUUCUCAAACGUCUGAAGUAUAUAUGGUAUGUAAAGGUUUGA
AGAAAUUAAUCGAUGAACCCAAUCCCGAUUGGUCUUCCAUCAAUGAAUCCUGG
AAAAACCUGUACGCAUUCCAGUCAUCAGAACAGGAAUUUGCCAGAGCAAAGAA
GGUUAGUACAUACUUUACCUUGACAGGUAUUCCCUCCCAAUUCAUUCCUGAUC
CUUUUGUAAACAUUGAGACUAUGCUACAAAUAUUCGGAGUACCCACGGGUGUG
UCUCAUGCGGCUGCCUUAAAAUCAUCUGAUAGACCUGCAGAUUUAUUGACCAU
UAGCCUUUUUUAUAUGGCGAUUAUAUCGUAUUAUAACAUCAAUCAUAUCAGA
GUAGGACCGAUACCUCCGAACCCCCCAUCAGAUGGAAUUGCACAAAAUGUGGG
GAUCGCUAUAACUGGUAUAAGCUUUUGGCUGAGUUUGAUGGAGAAAGACAUU
CCACUAUAUCAACAGUGUUUAGCAGUUAUCCAGCAAUCAUUCCCGAUUAGGUG
GGAGGCUGUUUCAGUAAAAGGAGGAUACAAGCAGAAGUGGAGUACUAGAGGU
GAUGGGCUCCCAAAAGAUACCCGAAUUUCAGACUCCUUGGCCCCAAUCGGGAA
CUGGAUCAGAUCUCUGGAAUUGGUCCGAAACCAAGUUCGUCUAAAUCCAUUCA
AUGAGAUCUUGUUCAAUCAGCUAUGUCGUACAGUGGAUAAUCAUUUGAAAUG
GUCAAAUUUGCGAAGAAACACAGGAAUGAUUGAAUGGAUCAAUAGACGAAUU
UCAAAAGAAGACCGGUCUAUACUGAUGUUGAAGAGUGACCUACACGAGGAAAA
CUCUUGGAGAGAUUAA.
(SEQ ID NO: 12)
AUGGAAGUCCACGAUUUUGAGACCGACGAGUUCAAUGAUUUCAAUG
AAGAUGACUAUGCCACAAGAGAAUUCCUGAAUCCCGAUGAGCGCAUGACGUAC
UUGAAUCAUGCUGAUUACAAUUUGAAUUCUCCUCUAAUUAGUGAUGAUAUUG
ACAAUUUGAUCAGGAAAUUCAAUUCUCUUCCGAUUCCCUCGAUGUGGGAUAGU
AAGAACUGGGAUGGAGUUCUUGAGAUGUUAACAUCAUGUCAAGCCAAUCCCAU
CUCAACAUCUCAGAUGCAUAAAUGGAUGGGAAGUUGGUUAAUGUCUGAUAAU
CAUGAUGCCAGUCAAGGGUAUAGUUUUUUACAUGAAGUGGACAAAGAGGCAG
AAAUAACAUUUGACGUGGUGGAGACCUUCAUCCGCGGCUGGGGCAACAAACCA
AUUGAAUACAUCAAAAAGGAAAGAUGGACUGACUCAUUCAAAAUUCUCGCUUA
UUUGUGUCAAAAGUUUUUGGACUUACACAAGUUGACAUUAAUCUUAAAUGCU
GUCUCUGAGGUGGAAUUGCUCAACUUGGCGAGGACUUUCAAAGGCAAAGUCAG
AAGAAGUUCUCAUGGAACGAACAUAUGCAGGAUUAGGGUUCCCAGCUUGGGUC
CUACUUUUAUUUCAGAAGGAUGGGCUUACUUCAAGAAACUUGAUAUUCUAAU
GGACCGAAACUUUCUGUUAAUGGUCAAAGAUGUGAUUAUAGGGAGGAUGCAA
ACGGUGCUAUCCAUGGUAUGUAGAAUAGACAACCUGUUCUCAGAGCAAGACAU
CUUCUCCCUUCUAAAUAUCUACAGAAUUGGAGAUAAAAUUGUGGAGAGGCAGG
GAAAUUUUUCUUAUGACUUGAUUAAAAUGGUGGAACCGAUAUGCAACUUGAA
GCUGAUGAAAUUAGCAAGAGAAUCAAGGCCUUUAGUCCCACAAUUCCCUCAUU
UUGAAAAUCAUAUCAAGACUUCUGUUGAUGAAGGGGCAAAAAUUGACCGAGG
UAUAAGAUUCCUCCAUGAUCAGAUAAUGAGUGUGAAAACAGUGGAUCUCACAC
UGGUGAUUUAUGGAUCGUUCAGACAUUGGGGUCAUCCUUUUAUAGAUUAUUA
CACUGGACUAGAAAAAUUACAUUCCCAAGUAACCAUGAAGAAAGAUAUUGAUG
UGUCAUAUGCAAAAGCACUUGCAAGUGAUUUAGCUCGGAUUGUUCUAUUUCAA
CAGUUCAAUGAUCAUAAAAAGUGGUUCGUGAAUGGAGACUUGCUCCCUCAUGA
UCAUCCCUUUAAAAGUCAUGUUAAAGAAAAUACAUGGCCCACAGCUGCUCAAG
UUCAAGAUUUUGGAGAUAAAUGGCAUGAACUUCCGCUGAUUAAAUGUUUUGA
AAUACCCGACUUACUAGACCCAUCGAUAAUAUACUCUGACAAAAGUCAUUCAA
UGAAUAGGUCAGAGGUGUUGAAACAUGUCCGAAUGAAUCCGAACACUCCUAUC
CCUAGUAAAAAGGUGUUGCAGACUAUGUUGGACACAAAGGCUACCAAUUGGAA
AGAAUUUCUUAAAGAGAUUGAUGAGAAGGGCUUAGAUGAUGAUGAUCUAAUU
AUUGGUCUUAAAGGAAAGGAGAGGGAACUGAAGUUGGCAGGUAGAUUUUUCU
CCCUAAUGUCUUGGAAAUUGCGAGAAUACUUUGUAAUUACCGAAUAUUUGAU
AAAGACUCAUUUCGUCCCUAUGUUUAAAGGCCUGACAAUGGCGGACGAUCUAA
CUGCAGUCAUUAAAAAGAUGUUAGAUUCCUCAUCCGGCCAAGGAUUGAAGUCA
UAUGAGGCAAUUUGCAUAGCCAAUCACAUUGAUUACGAAAAAUGGAAUAACCA
CCAAAGGAAGUUAUCAAACGGCCCAGUGUUCCGAGUUAUGGGCCAGUUCUUAG
GUUAUCCAUCCUUAAUCGAGAGAACUCAUGAAUUUUUUGAGAAAAGUCUUAU
AUACUACAAUGGAAGACCAGACUUGAUGCGUGUUCACAACAACACACUGAUCA
AUUCAACCUCCCAACGAGUUUGUUGGCAAGGACAAGAGGGUGGACUGGAAGGU
CUACGGCAAAAAGGAUGGACUAUCCUCAAUCUACUGGUUAUUCAAAGAGAGGC
UAAAAUCAGAAACACUGCUGUCAAAGUCUUGGCACAAGGUGAUAAUCAAGUUA
UUUGCACACAGUAUAAAACGAAGAAAUCGAGAAACGUUGUAGAAUUACAGGG
UGCUCUCAAUCAAAUGGUUUCUAAUAAUGAGAAAAUUAUGACUGCAAUCAAA
AUAGGGACAGGGAAGUUAGGACUUUUGAUAAAUGACGAUGAGACUAUGCAAU
CUGCAGAUUACUUGAAUUAUGGAAAAAUACCGAUUUUCCGUGGAGUGAUUAG
AGGGUUAGAGACCAAGAGAUGGUCACGAGUGACUUGUGUCACCAAUGACCAAA
UACCCACUUGUGCUAAUAUAAUGAGCUCAGUUUCCACAAAUGCUCUCACCGUA
GCUCAUUUUGCUGAGAACCCAAUCAAUGCCAUGAUACAGUACAAUUAUUUUGG
GACAUUUGCUAGACUCUUGUUGAUGAUGCAUGAUCCUGCUCUUCGUCAAUCAU
UGUAUGAAGUUCAAGAUAAGAUACCGGGCUUGCACAGUUCUACUUUCAAAUAC
GCCAUGUUGUAUUUGGACCCUUCCAUUGGAGGAGUGUCGGGCAUGUCUUUGUC
CAGGUUUUUGAUUAGAGCCUUCCCAGAUCCCGUAACAGAAAGUCUCUCAUUCU
GGAGAUUCAUCCAUGUACAUGCUCGAAGUGAGCAUCUGAAGGAGAUGAGUGCA
GUAUUUGGAAACCCCGAGAUAGCCAAGUUUCGAAUAACUCACAUAGACAAGCU
AGUAGAAGAUCCAACCUCUCUGAACAUCGCUAUGGGAAUGAGUCCAGCGAACU
UGUUAAAGACUGAGGUUAAAAAAUGCUUAAUCGAAUCAAGACAAACCAUCAG
GAACCAGGUGAUUAAGGAUGCAACCAUAUAUUUGUAUCAUGAAGAGGAUCGG
CUCAGAAGUUUCUUAUGGUCAAUAAAUCCUCUGUUCCCUAGAUUUUUAAGUGA
AUUCAAAUCAGGCACUUUUUUGGGAGUCGCAGACGGGCUCAUCAGUCUAUUUC
AAAAUUCUCGUACUAUUCGGAACUCCUUUAAGAAAAAGUAUCAUAGGGAAUU
GGAUGAUUUGAUUGUGAGGAGUGAGGUAUCCUCUUUGACACAUUUAGGGAAA
CUUCAUUUGAGAAGGGGAUCAUGUAAAAUGUGGACAUGUUCAGCUACUCAUGC
UGACACAUUAAGAUACAAAUCCUGGGGCCGUACAGUUAUUGGGACAACUGUAC
CCCAUCCAUUAGAAAUGUUGGGUCCACAACAUCGAAAAGAGACUCCUUGUGCA
CCAUGUAACACAUCAGGGUUCAAUUAUGUUUCUGUGCAUUGUCCAGACGGGAU
CCAUGACGUCUUUAGUUCACGGGGACCAUUGCCUGCUUAUCUAGGGUCUAAAA
CAUCUGAAUCUACAUCUAUUUUGCAGCCUUGGGAAAGGGAAAGCAAAGUCCCA
CUGAUUAAAAGAGCUACACGUCUUAGAGAUGCUAUCUCUUGGUUUGUUGAACC
CGACUCUAAACUAGCAAUGACUAUACUUUCUAACAUCCACUCUUUAACAGGCG
AAGAAUGGACCAAAAGGCAGCAUGGGUUCAAAAGAACAGGGUCUGCCCUUCAU
AGGUUUUCGACAUCUCGGAUGAGCCAUGGUGGGUUCGCAUCUCAGAGCACUGC
AGCAUUGACCAGGUUGAUGGCAACUACAGACACCAUGAGGGAUCUGGGAGAUC
AGAAUUUCGACUUUUUAUUCCAAGCAACGUUGCUCUAUGCUCAAAUUACCACC
ACUGUUGCAAGAGACGGAUGGAUCACCAGUUGUACAGAUCAUUAUCAUAUUGC
CUGUAAGUCCUGUUUGAGACCCAUAGAAGAGAUCACCCUGGACUCAAGUAUGG
ACUACACGCCCCCAGAUGUAUCCCAUGUGCUGAAGACAUGGAGGAAUGGGGAA
GGUUCGUGGGGACAAGAGAUAAAACAGAUCUAUCCUUUAGAAGGGAAUUGGA
AGAAUUUAGCACCUGCUGAGCAAUCCUAUCAAGUCGGCAGAUGUAUAGGUUUU
CUAUAUGGAGACUUGGCGUAUAGAAAAUCUACUCAUGCCGAGGACAGUUCUCU
AUUUCCUCUAUCUAUACAAGGUCGUAUUAGAGGUCGAGGUUUCUUAAAAGGG
UUGCUAGACGGAUUAAUGAGAGCAAGUUGCUGCCAAGUAAUACACCGGAGAAG
UCUGGCUCAUUUGAAGAGGCCGGCCAACGCAGUGUACGGAGGUUUGAUUUACU
UGAUUGAUAAAUUGAGUGUAUCACCUCCAUUCCUUUCUCUUACUAGAUCAGGA
CCUAUUAGAGACGAAUUAGAAACGAUUCCCCACAAGAUCCCAACCUCCUAUCC
GACAAGCAACCGUGAUAUGGGGGUGAUUGUCAGAAAUUACUUCAAAUACCAAU
GCCGUCUAAUUGAAAAGGGAAAAUACAGAUCACAUUAUUCACAAUUAUGGUU
AUUCUCAGAUGUCUUAUCCAUAGACUUCAUUGGACCAUUCUCUAUUUCCACCA
CCCUCUUGCAAAUCCUAUACAAGCCAUUUUUAUCUGGGAAAGAUAAGAAUGAG
UUGAGAGAGCUGGCAAAUCUUUCUUCAUUGCUAAGAUCAGGAGAGGGGUGGG
AAGACAUACAUGUGAAAUUCUUCACCAAGGACAUAUUAUUGUGUCCAGAGGAA
AUCAGACAUGCUUGCAAGUUCGGGAUUGCUAAGGAUAAUAAUAAAGACAUGA
GCUAUCCCCCUUGGGGAAGGGAAUCCAGAGGGACAAUUACAACAAUCCCUGUU
UAUUAUACGACCACCCCUUACCCAAAGAUGCUAGAGAUGCCUCCAAGAAUCCA
AAAUCCCCUGCUGUCCGGAAUCAGGUUGGGCCAAUUACCAACUGGCGCUCAUU
AUAAAAUUCGGAGUAUAUUACAUGGAAUGGGAAUCCAUUACAGGGACUUCUU
GAGUUGUGGAGACGGCUCCGGAGGGAUGACUGCUGCAUUACUACGAGAAAAUG
UGCAUAGCAGAGGAAUAUUCAAUAGUCUGUUAGAAUUAUCAGGGUCAGUCAU
GCGAGGCGCCUCUCCUGAGCCCCCCAGUGCCCUAGAAACUUUAGGAGGAGAUA
AAUCGAGAUGUGUAAAUGGUGAAACAUGUUGGGAAUAUCCAUCUGACUUAUG
UGACCCAAGGACUUGGGACUAUUUCCUCCGACUCAAAGCAGGCUUGGGGCUUC
AAAUUGAUUUAAUUGUAAUGGAUAUGGAAGUUCGGGAUUCUUCUACUAGCCU
GAAAAUUGAGACGAAUGUUAGAAAUUAUGUGCACCGGAUUUUGGAUGAGCAA
GGAGUUUUAAUCUACAAGACUUAUGGAACAUAUAUUUGUGAGAGCGAAAAGA
AUGCAGUAACAAUCCUUGGUCCCAUGUUCAAGACGGUCGACUUAGUUCAAACA
GAAUUUAGUAGUUCUCAAACGUCUGAAGUAUAUAUGGUAUGUAAAGGUUUGA
AGAAAUUAAUCGAUGAACCCAAUCCCGAUUGGUCUUCCAUCAAUGAAUCCUGG
AAAAACCUGUACGCAUUCCAGUCAUCAGAACAGGAAUUUGCCAGAGCAAAGAA
GGUUAGUACAUACUUUACCUUGACAGGUAUUCCCUCCCAAUUCAUUCCUGAUC
CUUUUGUAAACAUUGAGACUAUGCUACAAAUAUUCGGAGUACCCACGGGUGUG
UCUCAUGCGGCUGCCUUAAAAUCAUCUGAUAGACCUGCAGAUUUAUUGACCAU
UAGCCUUUUUUAUAUGGCGAUUAUAUCGUAUUAUAACAUCAAUCAUAUCAGA
GUAGGACCGAUACCUCCGAACCCCCCAUCAGAUGGAAUUGCACAAAAUGUGGG
GAUCGCUAUAACUGGUAUAAGCUUUUGGCUGAGUUUGAUGGAGAAAGACAUU
CCACUAUAUCAACAGUGUUUAGCAGUUAUCCAGCAAUCAUUCCCGAUUAGGUG
GGAGGCUGUUUCAGUAAAAGGAGGAUACAAGCAGAAGUGGAGUACUAGAGGU
GAUGGGCUCCCAAAAGAUACCCGAACUUCAGACUCCUUGGCCCCAAUCGGGAA
CUGGAUCAGAUCUCUGGAAUUGGUCCGAAACCAAGUUCGUCUAAAUCCAUUCA
AUGAGAUCUUGUUCAAUCAGCUAUGUCGUACAGUGGAUAAUCAUUUGAAAUG
GUCAAAUUUGCGAAGAAACACAGGAAUGAUUGAAUGGAUCAAUAGACGAAUU
UCAAAAGAAGACCGGUCUAUACUGAUGUUGAAGAGUGACCUACACGAGGAAAA
CUCUUGGAGAGAUUAA.
(SEQ ID NO: 18)
AUGGAUUUCGAUCUAAUAGAAGAUUCUGCUAACUGGGAAGAUGAUG
AGUCGGACUUUUUUUUGCGAGACAUUCUAUCUCAAGAAGAUCAGAUGUCUUAC
UUAAACACCGCAGACUAUAAUUUGAAUUCUCCACUCAUUUCCGAUGACAUGGU
UUAUAUAAUUAAAAGAAUGAAUCACGAGGAGGUUCCCCCCAUUUGGAGAUCUA
AGGAGUGGGAUUCUCCUCUUGAUAUGCUUAGAGGUUGUCAAGCUCAACCCAUG
UCUCACCAAGAGAUGCACAAUUGGUUUGGAACUUGGAUUCAAAAUGUUCAGCA
UGAUUCCGCCCAACCCUUCACAUUCUUAAAAGAGGUGGACAAAGAGUCAGAAA
UGACCUAUGACUUGGUCUCCACUUUUCUGAAGGGGUGGGUGGGGAAAGAUUAC
CCAUUCAAGUCAAAGAACAAAGAAAUUGAUAGUAUGGCAUUAGUCGGUCCUCU
UUGUCAAAAAUUCCUUGAUUUGCAUAAAAUCACUCUGAUAUUAAAUGCAGUU
UCUUUGGGGGAAACUAAAGAACUCCUAACCACUUUCAAGGGUAAAUACCGCAU
GAGUUGUGAAAACAUUCCUAUUGCUCGCUUAAGACUUCCAAGUCUAGGCCCUG
UAUUCAUGUGCAAAGGUUGGACUUAUAUCCACAAAGAAAGAGUGCUAAUGGA
UCGAACUUCCUUCUCAUGUGUAAAAGAUGUGAUAAUAGGACGAAUGCAGACAU
UUUUGUCUAUGAUCGGACGAUCGGAUAACAAGUUUAGUCCUGAUCAAAUCUAU
ACUCUGGCAAAUGUAUACAGAAUUGGAGAUAAAAUUUUAGAACAAUGCGGCA
AUAAGGCUUAUGAUUUGAUUAAAAUGAUUGAACCCAUCUGCAAUCUGAAAAU
GAUGGAAUUAGCUAGAUUGCAUCGUCCCAAGAUUCCGAAGUUCCCUCAUUUUG
AAGAGCAUGUCAAAGGCUCAGUGCAAGAAUUGACACAAAGGUCCAAUAGAAUA
CAAACAUUGUAUGAUCUGAUUAUGUCUAUGAAGGAUGUUGACCUCGUAUUAG
UGGUUUACGGAUCUUUCCGUCAUUGGGGUCACCCAUUCAUCGAUUAUUUUCAG
GGAUUGGAAAAAUUGCACACUCAAGUGAAUAUGGAAAAGCAUAUUGACAAGG
AGUACCCACAGCAAUUGGCCAGUGACUUAGCCAGACUAGUAUUAAAUAAACAA
UUCAGCGAAUCAAAAAAAUGGUUUGUAGACCCUUCAAAAAUGUCUCCCAAACA
CCCUUUUUAUGAACAUGUUAUCAAUAAGACAUGGCCUACAGCAGCUAAAAUUC
AAGACUUUGGAGACAAUUGGCACAAGCUCCCAUUGAUUCAAUGUUUUGAAAUA
CCAGACUUGAUAGAUCCAUCAGUGAUUUAUUCAGACAAAAGUCAUUCCAUGAA
CAAAAAGGAGGUGAUACAGCAUGUUCGGUCUAAGCCAAAUAUUCCCAUCCCAA
GCAACAAAGUUCUGCAAACAAUGCUUACCAAUAGAGCAACAAAUUGGAAAGCC
UUUCUGAAAGAUAUUGACGAAAAUGGACUGGAUGAUGACGAUUUAAUAAUCG
GAUUAAAGGGAAAAGAAAGAGAAUUGAAAAUAGCUGGGAGAUUCUUCUCAUU
AAUGUCAUGGAGAUUAAGGGAAUAUUUUGUGAUCACAGAGUACCUAAUCAAG
ACAUACUACGUUCCCUUGUUUAAGGGGUUAACUAUGGCAGAUGACCUCACAUC
GGUGAUCAAGAAGAUGAUGGACAGUUCAUCAGGUCAAGGGCUUGAUGAUUAU
UCAUCUGUUUGUUUAGCCAACCAUAUCGAUUAUGAGAAGUGGAAUAACCAUCA
GAGAAAGGAAUCGAAUGGUCCCAUCUUCAGAGUCAUGGGACAGUUUCUAGGUU
AUCCAUCACUUAUAGAGAGAACUCAUGAGUUUUUUGAGAAGAGCUUAAUUUA
CUAUAACGGGCGUCCAGAUUUGAUGACAAUUCGAAAUGGUACAUUGUGUAAU
UCCACGAAGCACCGAGUUUGCUGGAAUGGGCAGAAAGGAGGAUUGGAAGGACU
AAGACAAAAGGGAUGGAGUAUUGUGAAUCUCUUGGUUAUUCAAAGGGAAGCU
AAGAUCCGAAACACAGCAGUGAAGGUCUUAGCACAGGGAGAUAAUCAGGUUAU
AUGCACUCAAUACAAGACAAAGAAGACUAGAUCAGAGUUAGAGUUGAGAGCA
GUCUUACAUCAGAUGGCCGGGAAUAAUAACAAAAUUAUGGAAGAAAUUAAGA
GAGGUACAGAAAAGUUGGGCCUAAUCAUAAAUGAUGAUGAGACUAUGCAAUC
AGCAGACUAUUUGAACUAUGGGAAAAUUCCAAUUUUCCGAGGAGUCAUCAGAG
GACUAGAAACAAAGAGAUGGUCACGGGUGACAUGCGUCACAAAUGAUCAAAUC
CCGACGUGUGCCAAUUUGAUGUCCUCAGUCUCUACUAAUGCCUUGACUGUUGC
UCAUUUUGCUGAAAAUCCUAUAAAUGCAAUGAUUCAAUACAAUUACUUUGGA
ACCUUUGCACGAUUAUUGCUUUUUAUGCAUGACCCCGCAAUCCGGCAAUCCUU
GUAUAAGGUCCAAGAAAAAAUACCUGGUUUGCACACCAGAACAUUCAAAUACG
CCAUGUUGUACCUAGAUCCUUCAAUCGGAGGAGUAUGUGGUAUGGCAUUAUCU
CGUUUUUUAAUUAGAGCAUUUCCAGAUCCAGUAACAGAGAGUCUCUCAUUCUG
GAAGUUUAUCUAUGAACAUGCUUCUGAGCCUCAUCUAAAAAAGAUGGAAGUG
AUGUUCGAGACCCCCCAAGGUGCCAAAUUCAGAAUAGAACACAUCAACAAAUU
GUUAGAGGACCCUACCUCUCUAAACAUCUCAAUGGGUAUGAGUCCCGCAAAUU
UGUUAAAGAGUGAGGUCAAAAAAUGUUUAAUAGAAUCAAGAUCAUCCAUCAA
GAAUGAAAUCAUAAAAGAUGCUACUAUCUACAUGCAUCAAGAAGAAGAGAAG
CUUAGAGGAUUUUUGUGGUCCAUCAAACCAUUAUUCCCUCGUUUCUUGAGUGA
AUUCAAAGCUGGAACUUUUUUGGGAGUAUCUGAGGGCUUUAUAAAUUUAUUU
CAAAACUCACGUACCAUAAGAAACUCAUUUAAAAAGAGAUAUCACAAGGAUCU
UGAUGAAUUAAUAAUCAAGAGAGAAAUAUCGUCCUUGAGUCAUCUCGGAUCU
AUGCAUUAUCGAUUAGGGGAUAAUCAGAUAUGGUCCUGUUCUGCAUCUCGAGC
CGACAUAUUAAGAUACAAGUCAUGGACCAGGAAAGUUGUGGGAACUACAGUGC
CUCAUCCUUUGGAAAUGCACGGACCACCCUCAAAGAAGGAAAGACCUUGCCAA
UUGUGUAAUUCAUCCGGUCUCACAUAUAUUUCGGUACAUUGUCCAAAAGGAAU
UAUUGAUGUAUUCAAUAGAAGGGGACCUUUGCCGGCUUACUUGGGGUCCAAUA
CAUCUGAGUCUACAUCUAUUUUACAGCCGUGGGAAAAAGAGAGUAAAAUACCA
AUAAUUAAAAGAGCAACUAGAUUAAGGGACGCCAUUUCUUGGUUCAUCCCACC
CGAGUCUCCUCUGUCUACAUGCAUUUUGAAUAAUAUUCAGGCUUUAACAGGAG
AAGAUUGGAGCUCUAAACAACAUGGCUUUAAGAGGACAGGAUCAGCAUUACAU
AGAUUUUCCACCUCCCGGAUGAGCAAUGGAGGAUUUGCCUCACAAAGCCCGGC
CACCUUAACUCGCAUGAUUGCAACUACAGACACAAUGAGAGACUUUGGUACAA
AAAAUUAUGAUUUCAUGUUCCAAGCAUCUUUGUUAUAUGGACAGAUGACAAC
AAGUAUUUCAAGAUAUGGAACCCCAGGGUCUUGCACGGAUCAUUACCAUAUCA
GAUGUAAAGGAUGCAUUAGAGAGAUUGAAGAAGUAGAACUGAACACUAGUCU
AGAAUACAAGACGCCCGAUGUUUCUCACAUAUUGGAAAAAUGGAGGAACAAUA
CUGGAUCUUGGGGUCAUCAAAUCAAACAAUUGAAACCUGCCGAGGGAAACUGG
GAAUCAUUGUCUCCUGUAGAGCAAUCAUAUCAAGUUGCAAGAUGUAUUGGAU
UUCUUUAUGGUGAACUGACACACAAGAAAUCGAGACAGGCUGAUGAUAGUUCU
CUAUUCCCCUUGAGCAUCCAACUAAAAGUGAGAGGGAGAGGUUUCUUGCGAGG
CCUUCUAGACGGUUUGAUGAGAUCCAGUUGCUGUCAGGUAAUUCAUAGACGAA
GUGUUUCUACCUUAAAGAGACCUGCAAAUGCAGUUUACGGUGGCCUCAUAUAC
CUCAUUGAUAAACUAAGUGCCUCAAGUCCCUUCUUAUCACUUGUAAGAACCGG
ACCUAUUCGACAAGAAUUAGAACAGGUGCCACACAAGAUGUCUACAUCGUAUC
CAACGAACAUUAGGGACUUGGGCUCCAUUGUGAGAAAUUAUUUUAAAUAUCA
GUGUCGACCAGUUGAGAGAGGAAACUAUAAAACUUAUUAUAAUCAAAUAUGG
UUAUUUUCUGAUGUUUUGUCCACUGAAUUUAUAGGGCCAAUGCGUAUAUCCAG
CUCUCUCCUUAGACUCCUUUAUCGACCUUCUCUGACAAAGAAGGAUAGAGAAG
AAUUGAGGGAGUUAGCAGCACUAUCAUCUAAUUUACGAAGUGGAGAAGAUUG
GGAUGAUUUACAUAUCAAAUUUUUCUCAAACGAUCUCCUCUUUUGCUCACAAG
AGAUAAGACAUGCUUGCAAAUUUGGGAUUAAAAAAGAUAAUGAAGAUAUCAC
UUUUUACCCGAAUUGGGGAACAGAGUACAUUGGGAAUGUCACUGAUAUUCCUG
UGUUUUACCGAGCUCAAAAUGUCAAAAAGGAUAUUAGAGUACCCCCUCGGAUC
CAAAAUCCUUUAAUGUCAGGACUCAGGCUUGGACAACUGCCAACUGGGGCUCA
UUACAAAAUGAGAGCCAUUGUAUUUCGUCUUAAAAUUCCGUAUCAUGACUUUC
UGGAUUGCGGAGAUGGGUCAGGAGGAAUGACUGCAGCAUUACUCCGGUAUAA
UAGGACAUCCAGAGGAAUCUUUAACAGUUUGUUGGAUCUGUCAGACACAAUGC
UAAGAGGAUCCUCACCAGAACCUCCCAGUGCCCUUGAGACCCUGGGGGGAGAG
AGAGUAAGAUGUGUGAAUGGAGACAGUUGCUGGGAACAUCCUUCCGAUCUCAG
CGAUGAGAACACAUGGAAGUAUUUUCUCCAUUUAAAAAAGGGAUGUGGAAUG
AGUAUCAAUCUUAUUACCAUGGAUAUGGAAGUCCAGGAUUCUGUUAUAUCAU
ACAAAAUCGAGUCAUUGGUCAGACAGUAUGUUCCGGUUCUAUUGGAAUCCGAC
GGUUGCCUUAUUUAUAAAACAUAUGGAACAUAUAUUGCCACACAAGAAGACAA
UUCCCUGACUCUUAUAGGAUCACUUUUUCACUCUGUCCAACUUGUCCAAACGG
AUUUAAGCUCUAGUAAUACAUCCGAAUUGUACUUGGUAUGCAGGAGAUUGAA
GGAUUAUGUAGACACUCCCUUUGUUGAUUGGAUAGAAUUAUACGACAACUGG
GAGAAACAGUAUGCUUUCAGAAGCUUCAAAGAUGAAUUUCAAAGAGCUCAAUC
ACUUACCCCAGAGACAACCCUAAUAGGAAUACCUCCUCAAUUUGUACCGGAUC
CUGGAGUCAAUCUAGAGACCCUGUUUCAAAUAGCAGGGGUUCCCACCGGAGUU
GCGCAUGGAAUCACACAUCACAUAUUGCAGUCUAAGGAUAAAUUAAUAUCAAA
UGCCAUCGGAAGCAUGUGUGUUAUCUCUCAUUUCCACAUUAACACAAUACGGA
CCACAGAUAGUAUGCCUGGACCCCCAUCAGAUGGGGAUGUCAACAAAAUGUGU
UCAGCAUUGAUUGGGGCAUGUUUCUGGCUGGACUGGAUGGAAUCUGAUCUUA
ACUUAUACAAACAUUGUCUAAGAUCAAUAAUGAAGUCUAUGCCUGUGAGAUG
GUUUAGAACAUUAAAAAAUGAAAAAUGGUCGCAAAAAUGGGAUUGUAAAGGA
GAUGCAAUCCCAAAAGAUUCCAGAUUAGGAGACAGCCUUGCUAAUAUCGGCAA
UUGGAUAAGAGCUUGGGAAUUAAUUAGAAAUGGAAAUAAGUCUGAGCCUUUU
GAUUCAAUGGUAGCAGAAGCAUUGACAAAAUCUGUGGAUAAAUCACUUAGUU
GGAGGAAAAUCUCAAAGUCAACUGGAAUUCCGAGACUUCUAAACAGUGAUAUC
GAUUUGGUUGAUCAAUCUAUACUAAAUGUUCAGAUCGACAUCGUAGAAAAUC
AAGCUUGGCAAAAUUGA.
(SEQ ID NO: 24)
AUGGAUCUCAACCCGGUCGAUGAUGCUGCAGAGUUAUCAGAGGAGA
ACUUCUUCUCUGGGAAAUUGUCCAAGGAAUGCCGAAUUAGAGGAUUGAAUUCA
GUUGAUUACAAUCUGAACUCCCCGUUGGUGUCCGACGAUUUGACAUACUUGCU
GAACAAAUUUAAGGGAAAACCUGUGCCUAUCCGAUGGAAGAUGAAGAAAUGG
GACUCUAUUCUAGACCAACUGCGUAAACAUGAUCUGGAGUAUCUGAGACCCUC
GGAUCUACACCAAUGGUUUGCAGAAUGGAUGUUGUACUCAAAGCAUGGCAGUA
AGCAGGGAGAAGACUUCUUAAAGACAGUAGACGAAGAGGCUAGUGAUACGUU
UGAGGUUGUAAGGUCGUUCAUAAGGGGAUGGACUGGUGGAGAAAUCAACUUU
GUCAGGAAGAGCGGAAAGCAUAUGGGGUAUUGUGCGGAGUUGUGCCAGAAAU
UCUUGGAUUUGCAUAAAUUGACCCUCCUUGGAAAUGCCGCCACCGAUAAUGAG
UUGUUACAACUGAGCAAAACUUUCGGGGAUGACAAGAUUUACAAGAAACGGCU
AAUCAAACUUCCAAGUCUUGGUCGCGUCAUCUUUGAUUCUGGAUUCUUCAUUG
UCCUGGAUCAGCGAGUUCUAAUGGAUCGUAAUUUUAUGUUGAUGAUGAAAGA
CGUAAUAAUUGGAAGAAUGCAAACAGUUCUGUCCAUGAUUAGCAGAUGUGAU
GAUAAAUUCAGUUCCAAGGAUAUUGAUUUUCUAUUAAAGGUCUACUCAACAG
GCGAUAAGAUCAUAAGAAAGUUGGGUAACGAUGGGUAUGAGUUGAUCAAGAC
CGUAGAACCUAUGUGCAAUCUUCGUCUAUCAGACCUGGCCAGAAGGUUCCGUC
CACUCGUACCUCCUUUCCCUCAUUUCAGGAGACAUAUUGAAUCGACUGUAGAU
GAACUGAGUGCGAAGACUCCAUUGAUCAGGGAAUUGUUCUCAUUGAUUGAUAC
UUCUCCUAAUGUAGAUUCAACGUUGGUCGUUUACGGUUCAUUUCGUCAUUGGG
GUCAUCCUUUUAUUAAUUAUUUUGAAGGCCUGGAGAAAUUGCAUAAGCAAGU
GACAAUGGAAAAAGAAAUUGAUACCAAUUAUUCGGAAGCGUUGGCCAGUGAU
CUGGCUAGGAUAGUCUUGACUAAAGAGUUCAAUGAGAAAAAACAAUGGGCUG
UAGAUUACCACAGAGUGCCUACAAGCCAUCCCUUUAAAAACCACAUCCGAGAC
AACACAUGGCCAACAGCUGCAGUGAUCCAAGAUUUUGGAGAUCACUGGCAUGA
GCUCCCAUUAAUUCAAUGCUUCGACAUACCUGAUCUGAUUGAUCCAUCCAUUA
UCUACUCAGACAAGAGCCAUUCCAUGAAUCGAUCAGAGGUGUUAAAUCAUGUA
AGAACCAAGCCUCAUACACCGAUUCCAAGCAAGAAAGUUUUAGAGAGUAUGAU
CGACAAACCAGCGACAAAUUGGCUUGAGUUUCUUGAAGAGAUCGACAAAAAUG
GAUUAUCUGAUGAGGAUUUGGUGAUCGGAUUAAAAGGAAAAGAAAGAGAACU
GAAGAUUGCAGGUAGAUUUUUCUCAUUGAUGUCUUGGAAGCUUAGGGAAUAC
UUUGUUGUCACAGAGUAUUUGAUCAAGACUCACUUUGUGCCAUUGUUCCAUGG
GCUGACCAUGGCUGAUGAUAUGACAGCAGUGAUCAAAAAGAUGCUGGAAAGCU
CGUCUGGUCAAGGGCUAACAAACUACGACAGCGUUUGUAUUGCAAAUCAUAUU
GACUAUGAAAAAUGGAAUAAUCAUCAACGAAAAUUGUCCAAUGGACCUGUGU
UUAAAGUCAUGGGACAAUUCUUGGGUUAUCCAAAUUUGAUAUACCGGACACAU
GAGUUUUUUGAGAAAAGUUUAAUUUACUAUAAUGAAAGACCGGAUCUCAUGA
AAGUCAGAAAUGGGAUCUUAGAAAAUAGCACACAUCAGAGGGUUUGCUGGAA
UGGACAGGCAGGUGGUUUAGAAGGUUUGAGACAGAAAGGAUGGAGUAUUCUU
AAUUUGCUGGUCAUACAGAGAGAAGCGAAAAUCCGGAAUACCGCAGUUAAAGU
GCUAGCACAAGGCGACAAUCAAGUCAUAUGUACACAGUACAAGACCAAACAGU
AUCGAAACGAUAUCGAGUUGAGACAAGCCUUAAACCAAAUGGCAGCAAACAAU
GAUGUCAUCAUGAAGGCCAUAGAGUCAGGAACCAAUAAGUUGGGUUUGUUGA
UCAAUCAGGAUGAGACGAUGCAAUCUGCUGAUUAUCUGAACUAUGGGAAGGU
ACCUAUCUUUAGAGGAGUAAUCAGAGGGCUGGAAACAAAGCGAUGGUCCCGAG
UCACUUGUGUGACAAAUGACCAAUUACCAACGUGUGCAAAUCUCAUGUCAUCU
GUAUCUACAAAUGCUUUGACAGUGGCACAUUUCGAUGUACACCCAUUAAAUGC
AAUGAUCCAAUUUAACUUCUUCGGGAAUUUUGCUCGCCUUCUCUUGAUUAUGC
ACGAUCCGGCGAUUCGACAAUCAUUAAAUCAGUUGAAAGGACCAAACAUCAAU
GUCCAUUCAUACGGGUUCAAAGUUGCGAUGUUGUACUUGGACCCUUCAAUAGG
AGGAGUGUGUGGUACAGCCCUUUCUCGAUUUUUGAUUAGGAGUUUCCCUGACC
CUGUGACGGAGAGUCUCUCAUUUUGGAAGCUUAUCCAUCAUAGCACCAGUGAU
AUCAGAUUAAAAAAUCUCUCAGAACAGUUUGGCAAUCCUAAGAUAGCAGUUUU
UAGAGAAUCUCACAUAGAAAAGUUGUUGGAAGAUCCUACUAGUUUGAAUAUA
UCGAUGGGAAUGAGUGCUGCAAAUUUGUUGAAAACAGAGAUAAAGAAAAAUC
UCCUACAAAAGAAAUCAAGCAUAGGGAAUCAGAUAGUCAAAGACGCCGUAUAC
UACAUACACUCGGAAGAUGAGAAAUUGAGAACUUUUUUGUGGUCCAUUACUCC
AUUGUUUCCUCGGUUUCUGAGCGAAUUUAAAGCAGGGACGUUCAUGGGAGUA
GCGAGCAGCAUUGUAUCUUUGUUCCAAAACUCUCGAACCAUUCGAAAUGUUUU
CAGAGACUAUAUGAGUCAAACAAUUGAUGACUUGAUUGUCAAGAGUGAACUA
ACAUCUCUGGAGCAUUUGUCUAACUAUACUGACAGGAAAGGGAGUGGUGGGA
UCUGGAGUUGCUCUGCGGAACAGGCUGACAAACUUAGAAGAAUGUCCUGGAAG
CGUCCAGUGCUAGGGACAACAGUCCCUCACCCGUUGGAAAUGCACGGAAGGGG
AACACUGAAAUCCCCGCUAUCCAAGUGUUGCAAGGAGUCCCGAAUGGAUUAUA
UUUCUGUCCAUAUUCCUGAAGGAUUGAACAAAGUUUUGGACGGCCGAGGAAGC
CUACCAGCAUAUUUGGGUUCAAAAACAUCAGAGUCUACCUCAAUUCUUCAGCC
UUGGGAGAAGGAAAGCAAAAUUCCCAUCAUAAGAAGAGCUACUAGAUUACGA
GAUGCAAUUCAUUGGUUUGUGGAUCCGGACAGUAACUUAGCCCGAAGCAUAUU
GAACAAUAUUGAAUCCUUAACUGGAGAAAAGUGGGAAGGGGCUUUAAAAGGA
UAUAAGAGAACGGGUUCUGCCCUCCAUAGAUUUUCCACCUCGCGGGUAAGCCA
UGGUGGUUUUUCUUCACAAAGUCCUGCAUGUCUCACCAGAAUGAUGGCAACCA
CAGACACCAUGCGCGAUUAUGCUCAGCUCAAUUAUGACUUCAUGUUCCAAGCA
AGUCUUCUCUACUCUCAAAUGACCAGUUCCGUCAUUUUAAUGGGAACCACUGU
CUCAAACACUAUUCAUUUCCAUGUUACCUGUCGAAAAUGUAUCCGAGAAAUAA
CAGAACCGAUGUUAGAAAGUCCUAGGGAGUACAGAGGGAAGGACGUUCAUUU
AGUCCUGGCAAAAUGGAAGAAUUCUUCAAAUGGUUGGGGAGAAACCCUCCAAC
UACUAAAGCCAGUUGAGGGAGAUUGGGACACUAUCCCUCCUGUUGAAAAAUCU
UAUCAUGUGGGUAGGAUCUUGGGUUUUCUUUAUGGUGACCUAAAGAGUCAAA
AUUCCAGUCGUGCAGACGACAGUUCAAUCUUCCCCCUCAGCAUUCAAAUGAGA
UUAAGAGGUCGAGGCUUUUUACGAGGAAUCCUUGACGGAUUAGUCCGAGCUAG
UGCUUGUCAGGUUAUCCACAGGAGAAGUGUUGCUUUGCUUUCAAAACCAGCAA
AUGCAAUUUAUGGAGGAUUGAUUUACUUGAUAGACAAGAUAAGUGCAUCCAC
CUCGUUUACGACACUGUGUAGGGACGGACCCAUUAGAGAAGAACUAUCAUCUA
UACCUCACAAAAUUCCUACUUCGUAUCCAACUUCAAAUUCAGAUAUGGGACUC
CACAUUAGAAAUUAUUUGAAGUUUCAGUGCAAGACAGUCGAAUUGGGAAAAU
AUCAAUCUGAUAUAAAGGAUUUAUGGUUGUUCUCGGAUGUGAUGACGUCGAA
UAUUGCAGGACCCUUUGCAUUGUCUACGAAAAUUUUGAAGUGCUUAUACAAGC
CUGCUUUGUCCCAGAAAGAUCGAAACAAUAUCAGGAAGAUCAGCAAUUUCUCC
AAGAUGAUGCGAUCACAAUUGAGCUGGGACCCCACAAGCUCUGAAUUUAUUAC
AUCUCAAAUCCUAGUAUGCAAUGAGGAAAUUAGGCAUGCCUGCAAGUUUGGAA
UUCCAAAAUUAUCACUUAAGUUUGAUGAUCCAGUCUGGGGACCCGAGGAUUAU
GGAUUGAUCUGGUCCAUUCCAGUUGAUUAUUCCAGUCAAUCAGUUCCAAAGAA
CUUGAAGCCCUGUCCUAGAAUUCAAAACCCUAGCAUAUCUGGUUUUAGGUUAG
GACAACUUCCAACAGGAGCUCACUACAAGCUGAGAUCAAUCCUAAGGAAGAAA
AAUAUACAUUACAGGGAUGCACUGUGUGGGGGUGAUGGGUCAGGAGGUAUGA
CAGCGGCAGUCCUACGUUAUAAUCUAAAGGCCAGGGCCAUAUUUAAUAGCAUC
CUUGAUUUUGAUGGAUCAACAAUGAAGGGAGCUUCACCAGAUCCACCUAGUGC
AUUGGAAACAGUGGUGAAUGGCCGGACUCGGUGCGUCAACGCGGAGAGUUGUU
GGGAGAACCCGUCAGAUCUGAGUGAACAGAGAACAUGGGAUUAUUUCAAAUUC
UUGAAAACCCAUCACGGAUUAAAGAUAGACCUGAUUGUAUUGGACAUGGAAG
UGAGAGACUUUGCAAUCUCGGCAUCCAUAGAGAAGUGUGUCCGAAACAAUGUA
UCGUCAAUAUUGGAAGAAGACGGAGUCCUAAUAUACAAGACCUAUGGAUCAAC
AAUUGCAGCAGAAAGUAGCAAUGCAGUUGUAAAUAUUGGAGUAUUGUUUGAG
AGCGUAGAGUUGAUUCAGACGGAGUAUAGCAGUACAUCCACUUCAGAAGUGUA
CAUGUAUUGCCGAAAGAUUAAGAAGUUUGUGGACGCUCCUCACCCAGAUUGGA
UAUCACUUGAUUAUUAUUGGAGCAAAUUAUUUUGCUUUAGGAGUUACAAAGA
AGAAUUCUUCCGAUCCUACGAAGUGUCUCGUAAAGAAUCCCUAAAAGGAAUAC
CCAAUUCCUUCAUCCCUGACCCAUUGGUCAAUCUAGAGACAUUACUUCAGAUA
GCAGGAGUUCCUUCUGGGAUAUCACACCAGUUGGCAAUUGAUAUUAAGGAAUC
ACAGCUGACUCAGAUCACCGCUGCAAUGGUGCUUUGCGGAAUGAUCGCAAACU
ACACUUUGGAUGUGACAAAAAAGAGAGAUUCAUACAAUCCUCCUUCCGACGGA
AGAUUAAUUCGAAUGAGUGCUGCUUUAGUCGGAAUAAGUUUCUGGAUUUCCG
UCAAGUAUUAUGACAAAGAGUUGAAUUUCGAAUUGGAACAGAUAUUAUCCAG
AUCAUUUCCCAUAAGGUGGAUGCUUUCACGCAACUAUUUAUUCUGGACAACAA
AAAAGGGUUUUAGGAAUGCAAAAGACGUAAGACUAUCUGGGAAUAUGGCUAA
UAUAGGGAAUUGGAUUCGAUGCAUGGAGCUCCUUCAUCUCCCCCCGGGAUCCC
UGUCCAAGGAUGAGGUCACGACCACUUGUGGGAAAUACAUUAGAAGCUUGAAG
UAUUCUGUCAUACUCCAACAGACCGGGAUCAUUGAUUUGUGGAAAUCAAGGGU
CGCAAGUGAUGAUCGAUCAUUGAUGGAGGUUAAGACCGAAUUCAUUGAAUCU
GAGCAUUGGGUAGAUUAA.
(SEQ ID NO: 30)
AUGCUCGAUCCUGGAGAGGUCUAUGAUGACCCUAUUGACCCAAUCGA
GUUAGAGGCUGAACCCAGAGGAACCCCCAUUGUCCCCAACAUCUUGAGGAACU
CUGACUACAAUCUCAACUCUCCUUUGAUAGAAGAUCCUGCUAGACUAAUGUUA
GAAUGGUUAAAAACAGGGAAUAGACCUUAUCGGAUGACUCUAACAGACAAUU
GCUCCAGGUCUUUCAGAGUUUUGAAAGAUUAUUUCAAGAAGGUAGAUUUGGG
UUCUCUCAAGGUGGGCGGAAUGGCUGCACAGUCAAUGAUUUCUCUCUGGUUAU
AUGGUGCCCACUCUGAAUCCAACAGGAGCCGGAGAUGUAUAACAGACUUGGCC
CAUUUCUAUUCCAAGUCGUCCCCCAUAGAGAAGCUGUUGAAUCUCACGCUAGG
AAAUAGAGGGCUGAGAAUCCCCCCAGAGGGAGUGUUAAGUUGCCUUGAGAGGG
UUGAUUAUGAUAAUGCAUUUGGAAGGUAUCUUGCCAACACGUAUUCCUCUUAC
UUGUUCUUCCAUGUAAUCACCUUAUACAUGAACGCCCUAGACUGGGAUGAGGA
AAAGACCAUCCUAGCAUUAUGGAAAGAUUUAACCUCAGUGGACAUCGGGAAGG
ACUUGGUAAAGUUCAAAGACCAAAUAUGGGGACUGCUGAUCGUGACAAAGGAC
UUUGUUUACUCCCAAAGUUCCAAUUGUCUUUUUGACAGAAACUACACACUUAU
GCUAAAAGAUCUUUUCUUGUCUCGCUUCAACUCCUUAAUGGUCUUGCUCUCUC
CCCCAGAGCCCCGAUACUCAGAUGACUUGAUAUCUCAACUAUGCCAGCUGUAC
AUUGCUGGGGAUCAAGUCUUGUCUAUGUGUGGAAACUCCGGCUAUGAAGUCAU
CAAAAUAUUGGAGCCAUAUGUCGUGAAUAGUUUAGUCCAGAGAGCAGAAAAG
UUUAGGCCUCUCAUUCAUUCCUUGGGAGACUUUCCUGUAUUUAUAAAAGACAA
GGUAAGUCAACUUGAAGAGACGUUCGGUCCCUGUGCAAGAAGGUUCUUUAGGG
CUCUGGAUCAAUUCGACAACAUACAUGACUUGGUUUUUGUGUUUGGCUGUUAC
AGGCAUUGGGGGCACCCAUAUAUAGAUUAUCGAAAGGGUCUGUCAAAACUAUA
UGAUCAGGUUCACCUUAAAAAAGUGAUAGAUAAGUCCUACCAGGAGUGCUUAG
CAAGCGACCUAGCCAGGAGGAUCCUUAGAUGGGGUUUUGAUAAGUACUCCAAG
UGGUAUCUGGAUUCAAGAUUCCUAGCCCGAGACCACCCCUUGACUCCUUAUAU
CAAAACCCAAACAUGGCCACCCAAACAUAUUGUAGACUUGGUGGGGGAUACAU
GGCACAAGCUCCCGAUCACGCAGAUCUUUGAGAUUCCUGAAUCAAUGGAUCCG
UCAGAAAUAUUGGAUGACAAAUCACAUUCUUUCACCAGAACGAGACUAGCUUC
UUGGCUGUCAGAAAACCGAGGGGGGCCUGUUCCUAGCGAAAAAGUUAUUAUCA
CGGCCCUGUCUAAGCCGCCUGUCAAUCCCCGAGAGUUUCUGAGGUCUAUAGAC
CUCGGAGGAUUGCCAGAUGAAGACUUGAUAAUUGGCCUCAAGCCAAAGGAACG
GGAAUUGAAGAUUGAAGGUCGAUUCUUUGCUCUAAUGUCAUGGAAUCUAAGA
UUGUAUUUUGUCAUCACUGAAAAACUCUUGGCCAACUACAUCUUGCCACUUUU
UGACGCGCUGACUAUGACAGACAACCUGAACAAGGUGUUUAAAAAGCUGAUCG
ACAGGGUCACCGGGCAAGGGCUUUUGGACUAUUCAAGGGUCACAUAUGCAUUU
CACCUGGACUAUGAAAAGUGGAACAACCAUCAAAGAUUAGAGUCAACAGAGGA
UGUAUUUUCUGUCCUAGAUCAAGUGUUUGGAUUGAAGAGAGUGUUUUCUAGA
ACACACGAGUUUUUUCAAAAGGCCUGGAUCUAUUAUUCAGACAGAUCAGACCU
CAUCGGGUUACGGGAGGAUCAAAUAUACUGCUUAGAUGCGUCCAACGGCCCAA
CCUGUUGGAAUGGCCAGGAUGGCGGGCUAGAAGGCUUACGGCAGAAGGGCUGG
AGUCUAGUCAGCUUAUUGAUGAUAGAUAGAGAAUCUCAAAUCAGGAACACAA
GAACCAAAAUACUAGCUCAAGGAGACAACCAGGUUUUAUGUCCGACAUACAUG
UUGUCGCCAGGGCUAUCUCAAGAGGGGCUCCUCUAUGAAUUGGAGAGAAUAUC
AAGGAAUGCACUUUCGAUAUACAGAGCCGUCGAGGAAGGGGCAUCUAAGCUAG
GGCUGAUCAUCAAGAAAGAAGAGACCAUGUGUAGUUAUGACUUCCUCAUCUAU
GGAAAAACCCCUUUGUUUAGAGGUAACAUAUUGGUGCCUGAGUCCAAAAGAUG
GGCCAGAGUCUCUUGCGUCUCUAAUGACCAAAUAGUCAACCUCGCCAAUAUAA
UGUCGACAGUGUCCACCAAUGCGCUAACAGUGGCACAACACUCUCAAUCUUUG
AUCAAACCGAUGAGGGAUUUUCUGCUCAUGUCAGUACAGGCAGUCUUUCACUA
CCUGCUAUUUAGCCCAAUCUUAAAGGGAAGAGUUUACAAGAUUCUGAGCGCUG
AAGGGGAGAGCUUUCUCCUAGCCAUGUCAAGGAUAAUCUAUCUAGAUCCUUCU
UUGGGAGGGAUAUCUGGAAUGUCCCUCGGAAGAUUCCAUAUACGACAGUUCUC
AGACCCUGUCUCUGAAGGGUUAUCCUUCUGGAGAGAGAUCUGGUUAAGCUCCC
AAGAGUCCUGGAUUCACGCGUUGUGUCAAGAGGCUGGAAACCCAGAUCUUGGA
GAGAGAACACUCGAGAGCUUCACUCGCCUUCUAGAAGAUCCGACCACCUUAAA
UAUCAGAGGAGGGGCCAGUCCUACCAUUCUACUCAAGGAUGCAAUCAGAAAGG
CUUUAUAUGACGAGGUGGACAAGGUGGAAAAUUCAGAGUUUCGAGAGGCAAU
CCUGUUGUCCAAGACCCAUAGAGAUAAUUUUAUACUCUUCUUAAUAUCUGUUG
AGCCUCUGUUUCCUCGAUUUCUCAGUGAGCUAUUCAGUUCGUCUUUUUUGGGA
AUCCCCGAGUCAAUCAUUGGAUUGAUACAAAACUCCCGAACGAUAAGAAGGCA
GUUUAGAAAGAGUCUCUCAAAAACUUUAGAAGAAUCCUUCUACAACUCAGAGA
UCCACGGGAUUAGUCGGAUGACCCAGACACCUCAGAGGGUUGGGGGGGUGUGG
CCUUGCUCUUCAGAGAGGGCAGAUCAACUUAGGGAGAUCUCUUGGGGAAGAAA
AGUGGUAGGCACGACAGUUCCUCACCCUUCUGAGAUGUUGGGAUUACUUCCCA
AGUCCUCUAUUUCUUGCACUUGUGGAGCAACAGGAGGAGGCAAUCCUAGAGUU
UCUGUAUCAGUACUCCCGUCCUUUGAUCAGUCAUUUUUUUCACGAGGCCCCCU
AAAGGGAUACUUGGGCUCGUCCACCUCUAUGUCGACCCAGCUAUUCCAUGCAU
GGGAAAAAGUCACUAAUGUUCAUGUGGUGAAGAGAGCUCUAUCGUUAAAAGA
AUCUAUAAACUGGUUCAUUACUAGAGAUUCCAACUUGGCUCAAGCUCUAAUUA
GGAACAUUAUGUCUCUGACAGGCCCUGAUUUCCCUCUAGAGGAGGCCCCUGUC
UUCAAAAGGACGGGGUCAGCCUUGCAUAGGUUCAAGUCUGCCAGAUACAGCGA
AGGAGGGUAUUCUUCUGUCUGCCCGAACCUCCUCUCUCAUAUUUCUGUUAGUA
CAGACACCAUGUCUGAUUUGACCCAAGACGGGAAGAACUACGAUUUCAUGUUC
CAGCCAUUGAUGCUUUAUGCACAGACAUGGACAUCAGAGCUGGUACAGAGAGA
CACAAGGCUAAGAGACUCUACGUUUCAUUGGCACCUCCGAUGCAACAGGUGUG
UGAGACCCAUUGACGACGUGACCCUGGAGACCUCUCAGAUCUUCGAGUUUCCG
GAUGUGUCGAAAAGAAUAUCCAGAAUGGUUUCUGGGGCUGUGCCUCACUUCCA
GAGGCUUCCCGAUAUCCGUCUGAGACCAGGAGAUUUUGAAUCUCUAAGCGGUA
GAGAAAAGUCUCACCAUAUCGGAUCAGCUCAGGGGCUCUUAUACUCAAUCUUA
GUGGCAAUUCACGACUCAGGAUACAAUGAUGGAACCAUCUUCCCUGUCAACAU
AUACGGCAAGGUUUCCCCUAGAGACUAUUUGAGAGGGCUCGCAAGGGGAGUAU
UGAUAGGAUCCUCGAUUUGCUUCUUGACAAGAAUGACAAAUAUCAAUAUUAA
UAGACCUCUUGAAUUGGUCUCAGGGGUAAUCUCAUAUAUUCUCCUGAGGCUAG
AUAACCAUCCCUCCUUGUACAUAAUGCUCAGAGAACCGUCUCUUAGAGGAGAG
AUAUUUUCUAUCCCUCAGAAAAUCCCCGCCGCUUAUCCAACCACUAUGAAAGA
AGGCAACAGAUCAAUCUUGUGUUAUCUCCAACAUGUGCUACGCUAUGAGCGAG
AGAUAAUCACGGCGUCUCCAGAGAAUGACUGGCUAUGGAUCUUUUCAGACUUU
AGAAGUGCCAAAAUGACGUACCUAUCCCUCAUUACUUACCAGUCUCAUCUUCU
ACUCCAGAGGGUUGAGAGAAACCUAUCUAAGAGUAUGAGAGAUAACCUGCGAC
AAUUGAGUUCUUUGAUGAGGCAGGUGCUGGGCGGGCACGGAGAAGAUACCUU
AGAGUCAGACGACAACAUUCAACGACUGCUAAAAGACUCUUUACGAAGGACAA
GAUGGGUGGAUCAAGAGGUGCGCCAUGCAGCUAGAACCAUGACUGGAGAUUAC
AGCCCCAACAAGAAGGUGUCCCGUAAGGUAGGAUGUUCAGAAUGGGUCUGCUC
UGCUCAACAGGUUGCAGUCUCUACCUCAGCAAACCCGGCCCCUGUCUCGGAGC
UUGACAUAAGGGCCCUCUCUAAGAGGUUCCAGAACCCUUUGAUCUCGGGCUUG
AGAGUGGUUCAGUGGGCAACCGGUGCUCAUUAUAAGCUUAAGCCUAUUCUAGA
UGAUCUCAAUGUUUUCCCAUCUCUCUGCCUUGUAGUUGGGGACGGGUCAGGGG
GGAUAUCAAGGGCAGUCCUCAACAUGUUUCCAGAUGCCAAGCUUGUGUUCAAC
AGUCUUUUAGAGGUGAAUGACCUGAUGGCUUCCGGAACACAUCCACUGCCUCC
UUCAGCAAUCAUGAGGGGAGGAAAUGAUAUCGUCUCCAGAGUGAUAGAUCUU
GACUCAAUCUGGGAAAAACCGUCCGACUUGAGAAACUUGGCAACCUGGAAAUA
CUUCCAGUCAGUCCAAAAGCAGGUCAACAUGUCCUAUGACCUCAUUAUUUGCG
AUGCAGAAGUUACUGACAUUGCAUCUAUCAACCGGAUCACCCUGUUAAUGUCC
GAUUUUGCAUUGUCUAUAGAUGGACCACUCUAUUUGGUCUUCAAAACUUAUGG
GACUAUGCUAGUAAAUCCAAACUACAAGGCUAUUCAACACCUGUCAAGAGCGU
UCCCCUCGGUCACAGGGUUUAUCACCCAAGUAACUUCGUCUUUUUCAUCUGAG
CUCUACCUCCGAUUCUCCAAACGAGGGAAGUUUUUCAGAGAUGCUGAGUACUU
GACCUCUUCCACCCUUCGAGAAAUGAGCCUUGUGUUAUUCAAUUGUAGCAGCC
CCAAGAGUGAGAUGCAGAGAGCUCGUUCCUUGAACUAUCAGGAUCUUGUGAGA
GGAUUUCCUGAAGAAAUCAUAUCAAAUCCUUACAAUGAGAUGAUCAUAACUCU
GAUUGACAGUGAUGUAGAAUCUUUUCUAGUCCACAAGAUGGUUGAUGAUCUU
GAGUUACAGAGGGGAACUCUGUCUAAAGUGGCUAUCAUUAUAGCCAUCAUGAU
AGUUUUCUCCAACAGAGUCUUCAACGUUUCCAAACCCCUAACUGACCCCUCGU
UCUAUCCACCGUCUGAUCCCAAAAUCCUGAGGCACUUCAACAUAUGUUGCAGU
ACUAUGAUGUAUCUAUCUACUGCUUUAGGUGACGUCCCUAGCUUCGCAAGACU
UCACGACCUGUAUAACAGACCUAUAACUUAUUACUUCAGAAAGCAAGUCAUUC
GAGGGAACGUUUAUCUAUCUUGGAGUUGGUCCAACGACACCUCAGUGUUCAAA
AGGGUAGCCUGUAAUUCUAGCCUGAGUCUGUCAUCUCACUGGAUCAGGUUGAU
UUACAAGAUAGUGAAGACUACCAGACUCGUUGGCAGCAUCAAGGAUCUAUCCA
GAGAAGUGGAAAGACACCUUCAUAGGUACAACAGGUGGAUCACCCUAGAGGAU
AUCAGAUCUAGAUCAUCCCUACUAGACUACAGUUGCCUGUGA.

According to the embodiments of the present disclosure, the fourth RNA sequence has at least one of base sequences as set forth in SEQ ID NO: 35 to SEQ ID NO: 38.

(SEQ ID NO: 35)
AUGGAGAAGCCAGUAGUAAACGUAGACGUAGACCCCCAGAGUCCGUUUGU
CGUGCAACUGCAAAAAAGCUUCCCGCAAUUUGAGGUAGUAGCACAGCAGG
UCACUCCAAAUGACCAUGCUAAUGCCAGAGCAUUUUCGCAUCUGGCCAGU
AAACUAAUCGAGCUGGAGGUUCCUACCACAGCGACGAUCUUGGACAUAGG
CAGCGCACCGGCUCGUAGAAUGUUUUCCGAGCACCAGUAUCAUUGUGUCU
GCCCCAUGCGUAGUCCAGAAGACCCGGACCGCAUGAUGAAAUACGCCAGU
AAACUGGCGGAAAAAGCGUGCAAGAUUACAAACAAGAACUUGCAUGAGAA
GAUUAAGGAUCUCCGGACCGUACUUGAUACGCCGGAUGCUGAAACACCAU
CGCUCUGCUUUCACAACGAUGUUACCUGCAACAUGCGUGCCGAAUAUUCC
GUCAUGCAGGACGUGUAUAUCAACGCUCCCGGAACUAUCUAUCAUCAGGC
UAUGAAAGGCGUGCGGACCCUGUACUGGAUUGGCUUCGACACCACCCAGU
UCAUGUUCUCGGCUAUGGCAGGUUCGUACCCUGCGUACAACACCAACUGG
GCCGACGAGAAAGUCCUUGAAGCGCGUAACAUCGGACUUUGCAGCACAAA
GCUGAGUGAAGGUAGGACAGGAAAAUUGUCGAUAAUGAGGAAGAAGGAGU
UGAAGCCCGGGUCGCGGGUUUAUUUCUCCGUAGGAUCGACACUUUAUCCA
GAACACAGAGCCAGCUUGCAGAGCUGGCAUCUUCCAUCGGUGUUCCACUU
GAAUGGAAAGCAGUCGUACACUUGCCGCUGUGAUACAGUGGUGAGUUGCG
AAGGCUACGUAGUGAAGAAAAUCACCAUCAGUCCCGGGAUCACGGGAGAA
ACCGUGGGAUACGCGGUUACACACAAUAGCGAGGGCUUCUUGCUAUGCAA
AGUUACUGACACAGUAAAAGGAGAACGGGUAUCGUUCCCUGUGUGCACGU
ACAUCCCGGCCACCAUAUGCGAUCAGAUGACUGGUAUAAUGGCCACGGAU
AUAUCACCUGACGAUGCACAAAAACUUCUGGUUGGGCUCAACCAGCGAAU
UGUCAUUAACGGUAGGACUAACAGGAACACCAACACCAUGCAAAAUUACC
UUCUGCCGAUCAUAGCACAAGGGUUCAGCAAAUGGGCUAAGGAGCGCAAG
GAUGAUCUUGAUAACGAGAAAAUGCUGGGUACUAGAGAACGCAAGCUUAC
GUAUGGCUGCUUGUGGGCGUUUCGCACUAAGAAAGUACAUUCGUUUUAUC
GCCCACCUGGAACGCAGACCUGCGUAAAAGUCCCAGCCUCUUUUAGCGCU
UUUCCCAUGUCGUCCGUAUGGACGACCUCUUUGCCCAUGUCGCUGAGGCA
GAAAUUGAAACUGGCAUUGCAACCAAAGAAGGAGGAAAAACUGCUGCAGG
UCUCGGAGGAAUUAGUCAUGGAGGCCAAGGCUGCUUUUGAGGAUGCUCAG
GAGGAAGCCAGAGCGGAGAAGCUCCGAGAAGCACUUCCACCAUUAGUGGC
AGACAAAGGCAUCGAGGCAGCCGCAGAAGUUGUCUGCGAAGUGGAGGGGC
UCCAGGCGGACAUCGGAGCA.
(SEQ ID NO: 36)
GCAUUAGUUGAAACCCCGCGCGGUCACGUAAGGAUAAUACCUCAAGCAAA
UGACCGUAUGAUCGGACAGUAUAUCGUUGUCUCGCCAAACUCUGUGCUGA
AGAAUGCCAAACUCGCACCAGCGCACCCGCUAGCAGAUCAGGUUAAGAUC
AUAACACACUCCGGAAGAUCAGGAAGGUACGCGGUCGAACCAUACGACGC
UAAAGUACUGAUGCCAGCAGGAGGUGCCGUACCAUGGCCAGAAUUCCUAG
CACUGAGUGAGAGCGCCACGUUAGUGUACAACGAAAGAGAGUUUGUGAAC
CGCAAACUAUACCACAUUGCCAUGCAUGGCCCCGCCAAGAAUACAGAAGA
GGAGCAGUACAAGGUUACAAAGGCAGAGCUUGCAGAAACAGAGUACGUGU
UUGACGUGGACAAGAAGCGUUGCGUUAAGAAGGAAGAAGCCUCAGGUCUG
GUCCUCUCGGGAGAACUGACCAACCCUCCCUAUCAUGAGCUAGCUCUGGA
GGGACUGAAGACCCGACCUGCGGUCCCGUACAAGGUCGAAACAAUAGGAG
UGAUAGGCACACCGGGGUCGGGCAAGUCAGCUAUUAUCAAGUCAACUGUC
ACGGCACGAGAUCUUGUUACCAGCGGAAAGAAAGAAAAUUGUCGCGAAAU
UGAGGCCGACGUGCUAAGACUGAGGGGUAUGCAGAUUACGUCGAAGACAG
UAGAUUCGGUUAUGCUCAACGGAUGCCACAAAGCCGUAGAAGUGCUGUAC
GUUGACGAAGCGUUCGCGUGCCACGCAGGAGCACUACUUGCCUUGAUUGC
UAUCGUCAGGCCCCGCAAGAAGGUAGUACUAUGCGGAGACCCCAUGCAAU
GCGGAUUCUUCAACAUGAUGCAACUAAAGGUACAUUUCAAUCACCCUGAA
AAAGACAUAUGCACCAAGACAUUCUACAAGUAUAUCUCCCGGCGUUGCAC
ACAGCCAGUUACAGCUAUUGUAUCGACACUGCAUUACGAUGGAAAGAUGA
AAACCACGAACCCGUGCAAGAAGAACAUUGAAAUCGAUAUUACAGGGGCC
ACAAAGCCGAAGCCAGGGGAUAUCAUCCUGACAUGUUUCCGCGGGUGGGU
UAAGCAAUUGCAAAUCGACUAUCCCGGACAUGAAGUAAUGACAGCCGCGG
CCUCACAAGGGCUAACCAGAAAAGGAGUGUAUGCCGUCCGGCAAAAAGUC
AAUGAAAACCCACUGUACGCGAUCACAUCAGAGCAUGUGAACGUGUUGCU
CACCCGCACUGAGGACAGGCUAGUGUGGAAAACCUUGCAGGGCGACCCAU
GGAUUAAGCAGCCCACUAACAUACCUAAAGGAAACUUUCAGGCUACUAUA
GAGGACUGGGAAGCUGAACACAAGGGAAUAAUUGCUGCAAUAAACAGCCC
CACUCCCCGUGCCAAUCCGUUCAGCUGCAAGACCAACGUUUGCUGGGCGA
AAGCAUUGGAACCGAUACUAGCCACGGCCGGUAUCGUACUUACCGGUUGC
CAGUGGAGCGAACUGUUCCCACAGUUUGCGGAUGACAAACCACAUUCGGC
CAUUUACGCCUUAGACGUAAUUUGCAUUAAGUUUUUCGGCAUGGACUUGA
CAAGCGGACUGUUUUCUAAACAGAGCAUCCCACUAACGUACCAUCCCGCC
GAUUCAGCGAGGCCGGUAGCUCAUUGGGACAACAGCCCAGGAACCCGCAA
GUAUGGGUACGAUCACGCCAUUGCCGCCGAACUCUCCCGUAGAUUUCCGG
UGUUCCAGCUAGCUGGGAAGGGCACACAACUUGAUUUGCAGACGGGGAGA
ACCAGAGUUAUCUCUGCACAGCAUAACCUGGUCCCGGUGAACCGCAAUCU
UCCUCACGCCUUAGUCCCCGAGUACAAGGAGAAGCAACCCGGCCCGGUCA
AAAAAUUCUUGAACCAGUUCAAACACCACUCAGUACUUGUGGUAUCAGAG
GAAAAAAUUGAAGCUCCCCGUAAGAGAAUCGAAUGGAUCGCCCCGAUUGG
CAUAGCCGGUGCAGAUAAGAACUACAACCUGGCUUUCGGGUUUCCGCCGC
AGGCACGGUACGACCUGGUGUUCAUCAACAUUGGAACUAAAUACAGAAAC
CACCACUUUCAGCAGUGCGAAGACCAUGCGGCGACCUUAAAAACCCUUUC
GCGUUCGGCCCUGAAUUGCCUUAACCCAGGAGGCACCCUCGUGGUGAAGU
CCUAUGGCUACGCCGACCGCAACAGUGAGGACGUAGUCACCGCUCUUGCC
AGAAAGUUUGUCAGGGUGUCUGCAGCGAGACCAGAUUGUGUCUCAAGCAA
UACAGAAAUGUACCUGAUUUUCCGACAACUAGACAACAGCCGUACACGGC
AAUUCACCCCGCACCAUCUGAAUUGCGUGAUUUCGUCCGUGUAUGAGGGU
ACAAGAGAUGGAGUUGGAGCC.
(SEQ ID NO: 37)
GCGCCGUCAUACCGCACCAAAAGGGAGAAUAUUGCUGACUGUCAAGAGGA
AGCAGUUGUCAACGCAGCCAAUCCGCUGGGUAGACCAGGCGAAGGAGUCU
GCCGUGCCAUCUAUAAACGUUGGCCGACCAGUUUUACCGAUUCAGCCACG
GAGACAGGCACCGCAAGAAUGACUGUGUGCCUAGGAAAGAAAGUGAUCCA
CGCGGUCGGCCCUGAUUUCCGGAAGCACCCAGAAGCAGAAGCCUUGAAAU
UGCUACAAAACGCCUACCAUGCAGUGGCAGACUUAGUAAAUGAACAUAAC
AUCAAGUCUGUCGCCAUUCCACUGCUAUCUACAGGCAUUUACGCAGCCGG
AAAAGACCGCCUUGAAGUAUCACUUAACUGCUUGACAACCGCGCUAGACA
GAACUGACGCGGACGUAACCAUCUAUUGCCUGGAUAAGAAGUGGAAGGAA
AGAAUCGACGCGGCACUCCAACUUAAGGAGUCUGUAACAGAGCUGAAGGA
UGAAGAUAUGGAGAUCGACGAUGAGUUAGUAUGGAUCCAUCCAGACAGUU
GCUUGAAGGGAAGAAAGGGAUUCAGUACUACAAAAGGAAAAUUGUAUUCG
UACUUCGAAGGCACCAAAUUCCAUCAAGCAGCAAAAGACAUGGCGGAGAU
AAAGGUCCUGUUCCCUAAUGACCAGGAAAGUAAUGAACAACUGUGUGCCU
ACAUAUUGGGUGAGACCAUGGAAGCAAUCCGCGAAAAGUGCCCGGUCGAC
CAUAACCCGUCGUCUAGCCCGCCCAAAACGUUGCCGUGCCUUUGCAUGUA
UGCCAUGACGCCAGAAAGGGUCCACAGACUUAGAAGCAAUAACGUCAAAG
AAGUUACAGUAUGCUCCUCCACCCCCCUUCCUAAGCACAAAAUUAAGAAU
GUUCAGAAGGUUCAGUGCACGAAAGUAGUCCUGUUUAAUCCGCACACUCC
CGCAUUCGUUCCCGCCCGUAAGUACAUAGAAGUGCCAGAACAGCCUACCG
CUCCUCCUGCACAGGCCGAGGAGGCCCCCGAAGUUGUAGCGACACCGUCA
CCAUCUACAGCUGAUAACACCUCGCUUGAUGUCACAGACAUCUCACUGGA
UAUGGAUGACAGUAGCGAAGGCUCACUUUUUUCGAGCUUUAGCGGAUCGG
ACAACUCUAUUACUAGUAUGGACAGUUGGUCGUCAGGACCUAGUUCACUA
GAGAUAGUAGACCGAAGGCAGGUGGUGGUGGCUGACGUUCAUGCCGUCCA
AGAGCCUGCCCCUAUUCCACCGCCAAGGCUAAAGAAGAUGGCCCGCCUGG
CAGCGGCAAGAAAAGAGCCCACUCCACCGGCAAGCAAUAGCUCUGAGUCC
CUCCACCUCUCUUUUGGUGGGGUAUCCAUGUCCCUCGGAUCAAUUUUCGA
CGGAGAGACGGCCCGCCAGGCAGCGGUACAACCCCUGGCAACAGGCCCCA
CGGAUGUGCCUAUGUCUUUCGGAUCGUUUUCCGACGGAGAGAUUGAUGAG
CUGAGCCGCAGAGUAACUGAGUCCGAACCCGUCCUGUUUGGAUCAUUUGA
ACCGGGCGAAGUGAACUCAAUUAUAUCGUCCCGAUCAGCCGUAUCUUUUC
CACUACGCAAGCAGAGACGUAGACGCAGGAGCAGGAGGACUGAAUAC.
(SEQ ID NO: 38)
AUGAUGCCCACCGAAGCCAACAAAAGUAGGUACCAGUCUCGUAAAGUAGA
AAAUCAGAAAGCCAUAACCACUGAGCGACUACUGUCAGGACUACGACUGU
AUAACUCUGCCACAGAUCAGCCAGAAUGCUAUAAGAUCACCUAUCCGAAA
CCAUUGUACUCCAGUAGCGUACCGGCGAACUACUCCGAUCCACAGUUCGC
UGUAGCUGUCUGUAACAACUAUCUGCAUGAGAACUAUCCGACAGUAGCAU
CUUAUCAGAUUACUGACGAGUACGAUGCUUACUUGGAUAUGGUAGACGGG
ACAGUCGCCUGCCUGGAUACUGCAACCUUCUGCCCCGCUAAGCUUAGAAG
UUACCCGAAAAAACAUGAGUAUAGAGCCCCGAAUAUCCGCAGUGCGGUUC
CAUCAGCGAUGCAGAACACGCUACAAAAUGUGCUCAUUGCCGCAACUAAA
AGAAAUUGCAACGUCACGCAGAUGCGUGAACUGCCAACACUGGACUCAGC
GACAUUCAAUGUCGAAUGCUUUCGAAAAUAUGCAUGUAAUGACGAGUAUU
GGGAGGAGUUCGCUCGGAAGCCAAUUAGGAUUACCACUGAGUUUGUCACC
GCAUAUGUAGCUAGACUGAAAGGCCCUAAGGCCGCCGCACUAUUUGCAAA
GACGUAUAAUUUGGUCCCAUUGCAAGAAGUGCCUAUGGAUAGAUUCGUCA
UGGACAUGAAAAGAGACGUGAAAGUUACACCAGGCACGAAACACACAGAA
GAAAGACCGAAAGUACAAGUGAUACAAGCCGCAGAACCCCUGGCGACUGC
UUACUUAUGCGGGAUUCACCGGGAAUUAGUGCGUAGGCUUACGGCCGUCU
UGCUUCCAAACAUUCACACGCUUUUUGACAUGUCGGCGGAGGAUUUUGAU
GCAAUCAUAGCAGAACACUUCAAGCAAGGCGACCCGGUACUGGAGACGGA
UAUCGCAUCAUUCGACAAAAGCCAAGACGACGCUAUGGCGUUAACCGGUC
UGAUGAUCUUGGAGGACCUGGGUGUGGAUCAACCACUACUCGACUUGAUC
GAGUGCGCCUUUGGAGAAAUAUCAUCCACCCAUCUACCUACGGGUACUCG
UUUUAAAUUCGGGGCGAUGAUGAAAUCCGGAAUGUUCCUCACACUUUUUG
UCAACACAGUUUUGAAUGUCGUUAUCGCCAGCAGAGUACUAGAAGAGCGG
CUUAAAACGUCCAGAUGUGCAGCGUUCAUUGGCGACGACAACAUCAUACA
UGGAGUAGUAUCUGACAAAGAAAUGGCUGAGAGGUGCGCCACCUGGCUCA
ACAUGGAGGUUAAGAUCAUCGACGCAGUCAUCGGUGAGAGACCACCUUAC
UUCUGCGGCGGAUUUAUCUUGCAAGAUUCGGUUACUUCCACAGCGUGCCG
CGUGGCGGAUCCCCUGAAAAGGCUGUUUAAGUUGGGUAAACCGCUCCCAG
CCGACGACGAGCAAGACGAAGACAGAAGACGCGCUCUGCUAGAUGAAACA
AAGGCGUGGUUUAGAGUAGGUAUAACAGGCACUUUAGCAGUGGCCGUGAC
GACCCGGUAUGAGGUAGACAAUAUUACACCUGUCCUACUGGCAUUGAGAA
CUUUUGCCCAGAGCAAAAGAGCAUUCCAAGCCAUCAGAGGGGAAAUAAAG
CAUCUCUACGGUGGUCCUAAAUAG.

According to some specific embodiments of the present disclosure, the self-replicating RNA molecule includes one of the following six base sequence combinations:

sequence combination 1: the first RNA sequence has the base sequence as set forth in SEQ ID NO: 4, the second RNA sequence has the base sequence as set forth in SEQ ID NO: 5, and the third RNA sequence has the base sequence as set forth in SEQ ID NO: 6,

sequence combination 2: the first RNA sequence has the base sequence as set forth in SEQ ID NO: 10, the second RNA sequence has the base sequence as set forth in SEQ ID NO: 11, and the third RNA sequence has the base sequence as set forth in SEQ ID NO: 12,

sequence combination 3: the first RNA sequence has the base sequence as set forth in SEQ ID NO: 16, the second RNA sequence has the base sequence as set forth in SEQ ID NO: 17, and the third RNA sequence has the base sequence as set forth in SEQ ID NO: 18,

sequence combination 4: the first RNA sequence has the base sequence as set forth in SEQ ID NO: 22, the second RNA sequence has the base sequence as set forth in SEQ ID NO: 23, and the third RNA sequence has the base sequence as set forth in SEQ ID NO: 24,

sequence combination 5: the first RNA sequence has the base sequence as set forth in SEQ ID NO: 28, the second RNA sequence has the base sequence as set forth in SEQ ID NO: 29, and the third RNA sequence has the base sequence as set forth in SEQ ID NO: 30, and

sequence combination 6: the fourth RNA sequence has the base sequence as set forth in SEQ ID NO: 35 to SEQ ID NO: 38. This sequence combination is a self-replicating RNA sequence derived from positive-strand RNA virus, and its core complexes are NSP1, NSP2, NSP3, and NSP4.

According to an embodiment of the present disclosure, the self-replicating RNA molecule further includes a target molecule encoding at least one target molecule.

According to the embodiments of the present disclosure, the target molecule is suitable to serve as at least one of nucleic acid medicaments, protein medicaments, pathogen vaccines, tumor vaccines, and therapeutic agents for rare diseases. Thus, by utilizing the self-amplification characteristics of reRNA™ (the self-replicating RNA molecules and self-amplifying RNA molecules are interchangeable with reRNA™ in the present disclosure), the protein medicaments can be delivered by using very low dose of reRNA™. In addition, the self-amplifying reRNA™ can greatly improve the amount of protein synthesis and achieve the long-term protein expression. Based on these characteristics, the reRNA™ technology can be applied to various aspects of life science and medicine, such as nucleic acid drug delivery, protein drug delivery, pathogen vaccines, tumor vaccines, treatment of rare diseases and so on.

According to the embodiments of the present disclosure, the coding region of the target molecule can encode at least one of fluorescent protein, granulocyte-macrophage colony stimulating factor and ovalbumin OVA. Optionally, the fluorescent protein includes at least one of green fluorescent protein, red fluorescent protein, and yellow fluorescent protein. For example, by expressing ovalbumin in tumor cells, the immune system of animals can be activated to eliminate tumor cells, thereby achieving the purpose of treating cancer.

The self-replicating RNA molecule of the present disclosure may also include one or more modified nucleotides, thereby having advantages such as improved stability, resistance to degradation and elimination in vivo. The types of the modified nucleotide include, but are not limited to, 5-methylcytidine (m5C), 5-methyluridine (m5U), N6-methyladenosine (m6A), 2-thiouridine (s2U), 2-O-methyluridine (Um), 1-methyladenosine (m1A), 2-methyladenosine (m2A), 2-1-O-methyladenosine (Am), 2-methylthio-N6-methyladenosine (ms2m6A), N6-isopentenyladenosine (16A), 2-methylthio-N6-isopentenyladenosine (ms216A), N6-(cis-hydroxy isopentenyl)adenosine (io6A), 2-methylthio-N6-(cis-hydroxy isopentenyl)adenosine (ms2io6A), N6-glycyl carbamoyl adenosine (g6A), N6-threonyl carbamoyl adenosine (t6A), 2-methylthio-N6-threonyl carbamoyl adenosine (ms2t6A), N6-methyl-N6-threonyl carbamoyl adenosine (m6t6A), N6-hydroxy-norvalyl carbamoyl adenosine (hn6A), 2-methylthio-N6-hydroxy-norvalyl carbamoyl adenosine (ms2hn6A), 2-O-ribosyladenosine(phosphate) (Ar(p)), inosine (I), 1-methylinosine (m1I), 1,2-O-dimethylinosine (mIm), 3-methylcytidine (m3C), 2T-O-methylcytidine (Cm), 2-thiocyanine (s2C), N4-acetylcytidine (ac4C), 5-fonnylcytidine (f5C), 5,2-O-dimethylcytidine (m5Cm), N4 acetyl 2TO methyl cytidine (ac4Cm), lysidine (k2C), 1-methylguanosine (m1G), N2-methylguanosine (m2G), 7-methylguanosine (m7G), 2-O-methylguanosine (Gm), N2,N2-dimethylguanosine (m22G), N2,2-O-dimethylguanosine (m2Gm), N2,N2,2-O-trimethylguanosine (m22Gm), (2-O-ribosylguanosine (phosphate) (Gr(p)), wybutosine (yW), wybutosine peroxide (O2yW), hydroxyl wybutosine (OHyW), hydroxyl wybutosine with insufficient modification (OHyW*), wyosine (imG), methyl guanosine (mimG), queuosine (Q), epoxy queuosine (oQ), galactosyl-queuosine (galQ), mannose-queuosine (manQ), 7-cyano-7-deaza guanosine (preQo), 7-aminomethyl-7-deaza guanosine (preQi), gupurine glycoside (G*), dihydrouridine (D), 5,2-O-dimethyluridine (m5Um), 4-thiouridine (s4U), 5-methyl-2-thiouridine (m5s2U), 2-sulfur-2-O-methyluridine (s2Um), 3-(3-amino-3-carboxypropyl) uridine (acp3U), 5-hydroxyuridine (ho5U), 5-methoxyuridine (mo5U), uridine 5-oxoacetic acid (cmo5U), methyl uridine 5-oxoacetate (mcmo5U), 5-(carboxyhydroxymethyl) uridine (chm5U), 5-(methyl carboxyl hydroxymethyl) uridine (mchm5U), 5-methoxycarbonyl methyl uridine (mcm5U), S-methoxycarbonylmethyl-2-O-methyluridine (mcm5Um), 5-methoxycarbonylmethyl-2-thiouridine (mcm5s2U), 5-aminomethyl-2-thiouridine (nm5s2U), 5-methylaminomethyl uridine (mnm5U), 5-methylaminomethyl-2-thiouridine (mnm5s2U), 5-methylaminomethyl-2-seleno uridine (mnm5se2U), 5-carbamoylmethyl uridine (ncm5U), 5-carbamoylmethyl-2-O-methyluridine (ncm5Um), 5-carboxymethyl aminomethyl uridine (cmnm5U), 5-carboxymethyl aminomethyl-2-L-O methyluridine (cnmm5Um), 5-carboxymethylaminomethyl-2-thiouridine (cmnm5s2U), N6, N6-dimethyladenosine (m62A), 2-O-methylinosine (Tm), N4-methylcytidine (m4C), N4,2-O-dimethylcytidine (m4Cm), 5-hydroxymethylcytidine (hm5C), 3-methyluridine (m3U), 5-carboxymethyluridine (cm5U), N6, T-O-dimethyladenosine (m6Am), N6,N6,O-2-trimethyladenosine (rn62Am), N2,7-dimethylguanosine (m27G), N2,N2,7-trimethylguanosine (m227G), 3,2T-O-dimethyluridine (m3Um), 5-methyldihydrouridine (m5D), 5-formyl-2-O-methyl cytidine (f5Cm), 1,2-O-dimethylguanosine (m1Gm), 1,2-O-dimethyladenosine irinomethyluridine (mAm), S-taurino-methyl-2-thiouridine (tm5s2U), 4-demethylguanosine (imG-14), isoguanosine (imG2), N6-acetyladenosine (ac6A), hypoxanthine, inosine, 8-oxo-adenine, and the 7-substituted derivatives thereof, dihydrouracil, pseudouracil, 2-thiouracil, 4-thiouracil, 5-aminouracil, 5-(C1-C6)-alkyl uracil, 5-methyl uracil, 5-(C2-C6)-alkenyl uracil, 5-(C2-C6)-alkynyl uracil, 5-(hydroxymethyl) uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-denitrifying guanine, 8-azaguanine, 7-denitrogenation-7-substituted guanine, 7-denitrogenation-7-(C2-C6) alkynyl guanine, 7-denitrogenation-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-denitrifying purine, 7-azapurine-7-substituted purine, 7-azapurine-8-substituted purine, hydrogen (debased residue), m5C, m5U, m6A, s2U, W, or 2-O-methyl-U.

### Means of Preparing Self-Replicating RNA Molecules

In a second aspect of the present disclosure, the present disclosure provides a DNA molecule encoding the self-replicating RNA molecule described in any one of the preceding items. Those skilled in the art can understand that nucleic acids encoding self-replicating RNA molecules can be provided to cells in the form of DNA. In this way, the corresponding self-replicating RNA molecules can be synthesized in cells, and further, the self-replicating RNA molecules can be replicated and translated into corresponding proteins and corresponding target molecules in cells.

It should be noted that, for the DNA molecule mentioned in the specification and claims of the present disclosure, those skilled in the art should understand that the DNA molecule actually includes any one or two complementary double strands. For convenience, in this specification and the claims, although only one strand is given in most cases, another strand complementary to the mentioned one is actually disclosed. In addition, the specific sequences of DNA molecules in the present disclosure can be obtained according to the sequences of RNA molecules and the principle of base complementary pairing, and disclosure of one of them means that the other is also disclosed.

In a third aspect of the present disclosure, the present disclosure provides an expression vector carrying the aforementioned DNA molecule. The expression vector here is not particularly limited to a specific type, as long as it can replicate and express the corresponding RNA molecule in the host cells. As mentioned above, the nucleic acids encoding the self-replicating RNA molecules can be provided to cells in the form of DNA, allowing the corresponding self-replicating RNA molecules to be synthesized in cells, and further, the self- replicating RNA molecules can be replicated and translated into the corresponding proteins and target molecules in cells.

When the aforementioned DNA molecule is linked to the expression vector, it can be directly or indirectly linked to the control elements on the expression vector, as long as these control elements can control the translation and expression of the DNA molecule, that is, the DNA molecule is operably linked to the control elements. Of course, these control elements can be directly from the vector itself, or they can be exogenous, that is, not from the vector itself.

Herein, “operably linked” refers to a foreign gene is linked to a vector, enabling the control elements in the vector such as transcription control sequences and translation control sequences, etc. to exert their expected functions of regulating the transcription and translation of an exogenous gene. Common vectors can be, for example, plasmids, phages and the like.

In a fourth aspect of the present disclosure, the present disclosure provides a method for preparing the aforementioned self-replicating RNA molecule. The method includes: expressing, using the aforementioned DNA molecules or expression vectors, the aforementioned self-replicating RNA molecule in host cells; and collecting the self-replicating RNA molecule.

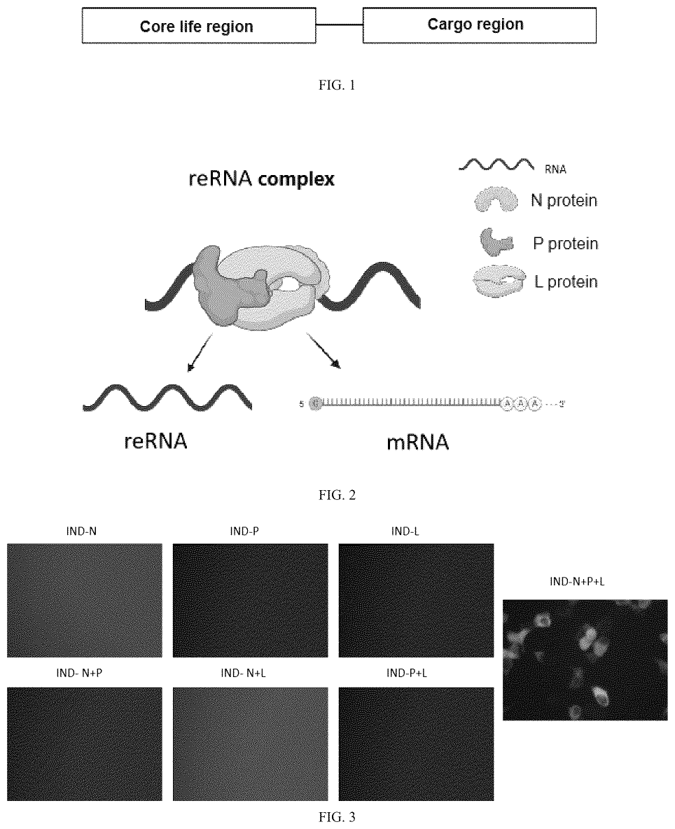

According to the embodiments of the present disclosure, by adopting the DNA molecules and expression vectors, the self-replicating RNA molecules expressed in the host cells such as animal cells usually are present in the form of complexes. For example, referring to FIG. 2, a complex is formed by the self-replicating RNA molecule together with the N protein, P protein, and L protein, thereby facilitating an initiation of self-coding efficiency of the self-replicating RNA molecules after being introduced into animal cells.

In a fifth aspect of the present disclosure, referring to FIG. 2, the present disclosure provides an RNA-protein complex, which includes the aforementioned self-replicating RNA molecules and proteins. The proteins include the N protein or a functional fragment thereof, the P protein or a functional fragment thereof, and the L protein or a functional fragment thereof. Therefore, the self-coding efficiency of the self-replicating RNA molecules can be conveniently initiated after they are introduced into animal cells.

In a sixth aspect of the present disclosure, the present disclosure provides a pharmaceutical composition, which includes the aforementioned self-replicating RNA molecule, DNA molecule, expression vector, or RNA-protein complex.

In a seventh aspect of the present disclosure, the present disclosure provides a method for expressing a target molecule in an individual. The method includes: administering to the individual the aforementioned self-replicating RNA molecule, DNA molecule, expression vector, RNA-protein complex, or pharmaceutical composition.

Additional aspects and advantages of the present disclosure will be set forth in part in the following description, and in part will be obvious from the following description, or may be learned by practice of the present disclosure. The specific techniques or conditions that are not specifically indicated in the present disclosure are shall be carried out according to the techniques or conditions described in the literature in the art or according to the product specification. The used reagents or instruments without indicating the manufacturers thereof are all conventional and commercially available products.

### Example 1. NPL Forms the Smallest Unit of reRNA™ Core Region to Achieve Intracellular Expression of reRNA™-Loaded Gene

In the present example, according to the information in the GeneBank database, the DNA sequence (base sequence as set forth in SEQ ID NO: 39) encoding N protein of vesicular stomatitis virus (VSV) Indiana strain (IND), the DNA sequence (base sequence as set forth in SEQ ID NO: 40) encoding P protein of vesicular stomatitis virus (VSV) Indiana strain (IND), the DNA sequence (base sequence as set forth in SEQ ID NO: 41) encoding L protein of vesicular stomatitis virus (VSV) Indiana strain (IND), and the DNA sequence encoding green fluorescent protein (GFP) were obtained. The amino acid sequences of the N protein, P protein, and L protein of the vesicular stomatitis virus (VSV) Indiana strain (IND) obtained after expression and translation were as set forth in SEQ ID NO: 1, SEQ ID NO: 2, and SEQ ID NO: 3, respectively. Specifically, reRNA™ DNA sequences carrying the following sequences were constructed respectively:

### IND-N-GFP;

IND-P-GFP;

### ND-L-GFP;

### IND-P+L-GFP;

### IND-N+L-GFP;

### IND-N+P-GFP; and

### IND-N+P+L-GFP.

It should be noted that, unless otherwise specified in the present disclosure, N represents the coding sequence of the N protein, P represents the coding sequence of the P protein, L represents the coding sequence of the L protein, IND represents that N, P or L followed by is derived from the Indian strain of vesicular stomatitis virus (VSV), NJ represents that N, P or L followed by is derived from the New Jersey strain of vesicular stomatitis virus (VSV), and GFP represents the coding sequence of the green fluorescent protein.

Furthermore, 293T cells were transfected in vitro with the DNA sequences constructed above, and cultured for 24 hours after the transfection. It was determined by observing the fluorescence signal of the green fluorescent protein whether the GFP coding sequence was correctly translated and expressed. The results were shown in FIG. 3. In FIG. 3, for convenience of description, the annotation of GFP is omitted. The results indicate that any individual protein or a combination of two proteins among the N protein, P protein, and L protein cannot initiate the expression of reRNA™ loaded gene (GFP), and the expression of the loaded gene GFP can only be initiated with the coexistence of the N protein, P protein and L protein (also referred to as “NPL” herein).

(SEQ ID NO: 39)
ATGTCTGTTACAGTCAAGAGAATCATTGACAACACAGTCGTAGTTCCAAA
ACTTCCTGCAAATGAGGATCCAGTGGAATACCCGGCAGATTACTTCAGAA
AATCAAAGGAGATTCCTCTTTACATCAATACTACAAAAAGTTTGTCAGAT
CTAAGAGGATATGTCTACCAAGGCCTCAAATCCGGAAATGTATCAATCAT
ACATGTCAACAGCTACTTGTATGGAGCATTAAAGGACATCCGGGGTAAGT
TGGATAAAGATTGGTCAAGTTTCGGAATAAACATCGGGAAAGCAGGGGAT
ACAATCGGAATATTTGACCTTGTATCCTTGAAAGCCCTGGACGGCGTACT
TCCAGATGGAGTATCGGATGCTTCCAGAACCAGCGCAGATGACAAATGGT
TGCCTTTGTATCTACTTGGCTTATACAGAGTGGGCAGAACACAAATGCCT
GAATACAGAAAAAAGCTCATGGATGGGCTGACAAATCAATGCAAAATGAT
CAATGAACAGTTTGAACCTCTTGTGCCAGAAGGTCGTGACATTTTTGATG
TGTGGGGAAATGACAGTAATTACACAAAAATTGTCGCTGCAGTGGACATG
TTCTTCCACATGTTCAAAAAACATGAATGTGCCTCGTTCAGATACGGAAC
TATTGTTTCCAGATTCAAAGATTGTGCTGCATTGGCAACATTTGGACACC
TCTGCAAAATAACCGGAATGTCTACAGAAGATGTAACGACCTGGATCTTG
AACCGAGAAGTTGCAGATGAAATGGTCCAAATGATGCTTCCAGGCCAAGA
AATTGACAAGGCCGATTCATACATGCCTTATTTGATCGACTTTGGATTGT
CTTCTAAGTCTCCATATTCTTCCGTCAAAAACCCTGCCTTCCACTTCTGG
GGGCAATTGACAGCTCTTCTGCTCAGATCCACCAGAGCAAGGAATGCCCG
ACAGCCTGATGACATTGAGTATACATCTCTTACTACAGCAGGTTTGTTGT
ACGCTTATGCAGTAGGATCCTCTGCCGACTTGGCACAACAGTTTTGTGTT
GGAGATAACAAATACACTCCAGATGATAGTACCGGAGGATTGACGACTAA
TGCACCGCCACAAGGCAGAGATGTGGTCGAATGGCTCGGATGGTTTGAAG
ATCAAAACAGAAAACCGACTCCTGATATGATGCAGTATGCGAAAAGAGCA
GTCATGTCACTGCAAGGCCTAAGAGAGAAGACAATTGGCAAGTATGCTAA
GTCAGAATTTGACAAATGA.
(SEQ ID NO: 40)
ATGGATAATCTCACAAAAGTTCGTGAGTATCTCAAGTCCTATTCTCGTCT
GGATCAGGCGGTAGGAGAGATAGATGAGATCGAAGCACAACGAGCTGAAA
AGTCCAATTATGAGTTGTTCCAAGAGGATGGAGTGGAAGAGCATACTAAG
CCCTCTTATTTTCAGGCAGCAGATGATTCTGACACAGAATCTGAACCAGA
AATTGAAGACAATCAAGGCTTGTATGCACCAGATCCAGAAGCTGAGCAAG
TTGAAGGCTTTATACAGGGGCCTTTAGATGACTATGCAGATGAGGAAGTG
GATGTTGTATTTACTTCGGACTGGAAACAGCCTGAGCTTGAATCTGACGA
GCATGGAAAGACCTTACGGTTGACATCGCCAGAGGGTTTAAGTGGAGAGC
AGAAATCCCAGTGGCTTTCGACGATTAAAGCAGTCGTGCAAAGTGCCAAA
TACTGGAATCTGGCAGAGTGCACATTTGAAGCATCGGGAGAAGGGGTCAT
TATGAAGGAGCGCCAGATAACTCCGGATGTATATAAGGTCACTCCAGTGA
TGAACACACATCCGTCCCAATCAGAAGCAGTATCAGATGTTTGGTCTCTC
TCAAAGACATCCATGACTTTCCAACCCAAGAAAGCAAGTCTTCAGCCTCT
CACCATATCCTTGGATGAATTGTTCTCATCTAGAGGAGAGTTCATCTCTG
TCGGAGGTGACGGACGAATGTCTCATAAAGAGGCCATCCTGCTCGGCCTG
AGATACAAAAAGTTGTACAATCAGGCGAGAGTCAAATATTCTCTGTAG.
(SEQ ID NO: 41)
ATGGAAGTCCACGATTTTGAGACCGACGAGTTCAATGATTTCAATGAAGA
TGACTATGCCACAAGAGAATTCCTGAATCCCGATGAGCGCATGACGTACT
TGAATCATGCTGATTACAACCTGAATTCTCCTCTAATTAGTGATGATATT
GACAATTTAATCAGGAAATTCAATTCTCTTCCAATTCCCTCGATGTGGGA
TAGTAAGAACTGGGATGGAGTTCTTGAGATGTTAACGTCATGTCAAGCCA
ATCCCATCCCAACATCTCAGATGCATAAATGGATGGGAAGTTGGTTAATG
TCTGATAATCATGATGCCAGTCAAGGGTATAGTTTTTTACATGAAGTGGA
CAAAGAGGCAGAAATAACATTTGACGTGGTGGAGACCTTCATCCGCGGCT
GGGGCAACAAACCAATTGAATACATCAAAAAGGAAAGATGGACTGACTCA
TTCAAAATTCTCGCTTATTTGTGTCAAAAGTTTTTGGACTTACACAAGTT
GACATTAATCTTAAATGCTGTCTCTGAGGTGGAATTGCTCAACTTGGCGA
GGACTTTCAAAGGCAAAGTCAGAAGAAGTTCTCATGGAACGAACATATGC
AGGATTAGGGTTCCCAGCTTGGGTCCTACTTTTATTTCAGAAGGATGGGC
TTACTTCAAGAAACTTGATATTCTAATGGACCGAAACTTTCTGTTAATGG
TCAAAGATGTGATTATAGGGAGGATGCAAACGGTGCTATCCATGGTATGT
AGAATAGACAACCTGTTCTCAGAGCAAGACATCTTCTCCCTTCTAAATAT
CTACAGAATTGGAGATAAAATTGTGGAGAGGCAGGGAAATTTTTCTTATG
ACTTGATTAAAATGGTGGAACCGATATGCAACTTGAAGCTGATGAAATTA
GCAAGAGAATCAAGGCCTTTAGTCCCACAATTCCCTCATTTTGAAAATCA
TATCAAGACTTCTGTTGATGAAGGGGCAAAAATTGACCGAGGTATAAGAT
TCCTCCATGATCAGATAATGAGTGTGAAAACAGTGGATCTCACACTGGTG
ATTTATGGATCGTTCAGACATTGGGGTCATCCTTTTATAGATTATTACAC
TGGACTAGAAAAATTACATTCCCAAGTAACCATGAAGAAAGATATTGATG
TGTCATATGCAAAAGCACTTGCAAGTGATTTAGCTCGGATTGTTCTATTT
CAACAGTTCAATGATCATAAAAAGTGGTTCGTGAATGGAGACTTGCTCCC
TCATGATCATCCCTTTAAAAGTCATGTTAAAGAAAATACATGGCCCACAG
CTGCTCAAGTTCAAGATTTTGGAGATAAATGGCATGAACTTCCGCTGATT
AAATGTTTTGAAATACCCGACTTACTAGACCCATCGATAATATACTCTGA
CAAAAGTCATTCAATGAATAGGTCAGAGGTGTTGAAACATGTCCGAATGA
ATCCGAACACTCCTATCCCTAGTAAAAAGGTGTTGCAGACTATGTTGGAC
ACAAAGGCTACCAATTGGAAAGAATTTCTTAAAGAGATTGATGAGAAGGG
CTTAGATGATGATGATCTAATTATTGGTCTTAAAGGAAAGGAGAGGGAAC
TGAAGTTGGCAGGTAGATTTTTCTCCCTAATGTCTTGGAAATTGCGAGAA
TACTTTGTAATTACCGAATATTTGATAAAGACTCATTTCGTCCCTATGTT
TAAAGGCCTGACAATGGCGGACGATCTAACTGCAGTCATTAAAAAGATGT
TAGATTCCTCATCCGGCCAAGGATTGAAGTCATATGAGGCAATTTGCATA
GCCAATCACATTGATTACGAAAAATGGAATAACCACCAAAGGAAGTTATC
AAACGGCCCAGTGTTCCGAGTTATGGGCCAGTTCTTAGGTTATCCATCCT
TAATCGAGAGAACTCATGAATTTTTTGAGAAAAGTCTTATATACTACAAT
GGAAGACCAGACTTGATGCGTGTTCACAACAACACACTGATCAATTCAAC
CTCCCAACGAGTTTGTTGGCAAGGACAAGAGGGTGGACTGGAAGGTCTAC
GGCAAAAAGGATGGAGTATCCTCAATCTACTGGTTATTCAAAGAGAGGCT
AAAATCAGAAACACTGCTGTCAAAGTCTTGGCACAAGGTGATAATCAAGT
TATTTGCACACAGTATAAAACGAAGAAATCGAGAAACGTTGTAGAATTAC
AGGGTGCTCTCAATCAAATGGTTTCTAATAATGAGAAAATTATGACTGCA
ATCAAAATAGGGACAGGGAAGTTAGGACTTTTGATAAATGACGATGAGAC
TATGCAATCTGCAGATTACTTGAATTATGGAAAAATACCGATTTTCCGTG
GAGTGATTAGAGGGTTAGAGACCAAGAGATGGTCACGAGTGACTTGTGTC
ACCAATGACCAAATACCCACTTGTGCTAATATAATGAGCTCAGTTTCCAC
AAATGCTCTCACCGTAGCTCATTTTGCTGAGAACCCAATCAATGCCATGA
TACAGTACAATTATTTTGGGACATTTGCTAGACTCTTGTTGATGATGCAT
GATCCTGCTCTTCGTCAATCATTGTATGAAGTTCAAGATAAGATACCGGG
CTTGCACAGTTCTACTTTCAAATACGCCATGTTGTATTTGGACCCTTCCA
TTGGAGGAGTGTCGGGCATGTCTTTGTCCAGGTTTTTGATTAGAGCCTTC
CCAGATCCCGTAACAGAAAGTCTCTCATTCTGGAGATTCATCCATGTACA
TGCTCGAAGTGAGCATCTGAAGGAGATGAGTGCAGTATTTGGAAACCCCG
AGATAGCCAAGTTTCGAATAACTCACATAGACAAGCTAGTAGAAGATCCA
ACCTCTCTGAACATCGCTATGGGAATGAGTCCAGCGAACTTGTTAAAGAC
TGAGGTTAAAAAATGCTTAATCGAATCAAGACAAACCATCAGGAACCAGG
TGATTAAGGATGCAACCATATATTTGTATCATGAAGAGGATCGGCTCAGA
AGTTTCTTATGGTCAATAAATCCTCTGTTCCCTAGATTTTTAAGTGAATT
CAAATCAGGCACTTTTTTGGGAGTCGCAGACGGGCTCATCAGTCTATTTC
AAAATTCTCGTACTATTCGGAACTCCTTTAAGAAAAAGTATCATAGGGAA
TTGGATGATTTGATTGTGAGGAGTGAGGTATCCTCTTTGACACATTTAGG
GAAACTTCATTTGAGAAGGGGATCATGTAAAATGTGGACATGTTCAGCTA
CTCATGCTGACACATTAAGATACAAATCCTGGGGCCGTACAGTTATTGGG
ACAACTGTACCCCATCCATTAGAAATGTTGGGTCCACAACATCGAAAAGA
GACTCCTTGTGCACCATGTAACACATCAGGGTTCAATTATGTTTCTGTGC
ATTGTCCAGACGGGATCCATGACGTCTTTAGTTCACGGGGACCATTGCCT
GCTTATCTAGGGTCTAAAACATCTGAATCTACATCTATTTTGCAGCCTTG
GGAAAGGGAAAGCAAAGTCCCACTGATTAAAAGAGCTACACGTCTTAGAG
ATGCTATCTCTTGGTTTGTTGAACCCGACTCTAAACTAGCAATGACTATA
CTTTCTAACATCCACTCTTTAACAGGCGAAGAATGGACCAAAAGGCAGCA
TGGGTTCAAAAGAACAGGGTCTGCCCTTCATAGGTTTTCGACATCTCGGA
TGAGCCATGGTGGGTTCGCATCTCAGAGCACTGCAGCATTGACCAGGTTG
ATGGCAACTACAGACACCATGAGGGATCTGGGAGATCAGAATTTCGACTT
TTTATTCCAAGCAACGTTGCTCTATGCTCAAATTACCACCACTGTTGCAA
GAGACGGATGGATCACCAGTTGTACAGATCATTATCATATTGCCTGTAAG
TCCTGTTTGAGACCCATAGAAGAGATCACCCTGGACTCAAGTATGGACTA
CACGCCCCCAGATGTATCCCATGTGCTGAAGACATGGAGGAATGGGGAAG
GTTCGTGGGGACAAGAGATAAAACAGATCTATCCTTTAGAAGGGAATTGG
AAGAATTTAGCACCTGCTGAGCAATCCTATCAAGTCGGCAGATGTATAGG
TTTTCTATATGGAGACTTGGCGTATAGAAAATCTACTCATGCCGAGGACA
GTTCTCTATTTCCTCTATCTATACAAGGTCGTATTAGAGGTCGAGGTTTC
TTAAAAGGGTTGCTAGACGGATTAATGAGAGCAAGTTGCTGCCAAGTAAT
ACACCGGAGAAGTCTGGCTCATTTGAAGAGGCCGGCCAACGCAGTGTACG
GAGGTTTGATTTACTTGATTGATAAATTGAGTGTATCACCTCCATTCCTT
TCTCTTACTAGATCAGGACCTATTAGAGACGAATTAGAAACGATTCCCCA
CAAGATCCCAACCTCCTATCCGACAAGCAACCGTGATATGGGGGTGATTG
TCAGAAATTACTTCAAATACCAATGCCGTCTAATTGAAAAGGGAAAATAC
AGATCACATTATTCACAATTATGGTTATTCTCAGATGTCTTATCCATAGA
CTTCATTGGACCATTCTCTATTTCCACCACCCTCTTGCAAATCCTATACA
AGCCATTTTTATCTGGGAAAGATAAGAATGAGTTGAGAGAGCTGGCAAAT
CTTTCTTCATTGCTAAGATCAGGAGAGGGGTGGGAAGACATACATGTGAA
ATTCTTCACCAAGGACATATTATTGTGTCCAGAGGAAATCAGACATGCTT
GCAAGTTCGGGATTGCTAAGGATAATAATAAAGACATGAGCTATCCCCCT
TGGGGAAGGGAATCCAGAGGGACAATTACAACAATCCCTGTTTATTATAC
GACCACCCCTTACCCAAAGATGCTAGAGATGCCTCCAAGAATCCAAAATC
CCCTGCTGTCCGGAATCAGGTTGGGCCAATTACCAACTGGCGCTCATTAT
AAAATTCGGAGTATATTACATGGAATGGGAATCCATTACAGGGACTTCTT
GAGTTGTGGAGACGGCTCCGGAGGGATGACTGCTGCATTACTACGAGAAA
ATGTGCATAGCAGAGGAATATTCAATAGTCTGTTAGAATTATCAGGGTCA
GTCATGCGAGGCGCCTCTCCTGAGCCCCCCAGTGCCCTAGAAACTTTAGG
AGGAGATAAATCGAGATGTGTAAATGGTGAAACATGTTGGGAATATCCAT
CTGACTTATGTGACCCAAGGACTTGGGACTATTTCCTCCGACTCAAAGCA
GGCTTGGGGCTTCAAATTGATTTAATTGTAATGGATATGGAAGTTCGGGA
TTCTTCTACTAGCCTGAAAATTGAGACGAATGTTAGAAATTATGTGCACC
GGATTTTGGATGAGCAAGGAGTTTTAATCTACAAGACTTATGGAACATAT
ATTTGTGAGAGCGAAAAGAATGCAGTAACAATCCTTGGTCCCATGTTCAA
GACGGTCGACTTAGTTCAAACAGAATTTAGTAGTTCTCAAACGTCTGAAG
TATATATGGTATGTAAAGGTTTGAAGAAATTAATCGATGAACCCAATCCC
GATTGGTCTTCCATCAATGAATCCTGGAAAAACCTGTACGCATTCCAGTC
ATCAGAACAGGAATTTGCCAGAGCAAAGAAGGTTAGTACATACTTTACCT
TGACAGGTATTCCCTCCCAATTCATTCCTGATCCTTTTGTAAACATTGAG
ACTATGCTACAAATATTCGGAGTACCCACGGGTGTGTCTCATGCGGCTGC
CTTAAAATCATCTGATAGACCTGCAGATTTATTGACCATTAGCCTTTTTT
ATATGGCGATTATATCGTATTATAACATCAATCATATCAGAGTAGGACCG
ATACCTCCGAACCCCCCATCAGATGGAATTGCACAAAATGTGGGGATCGC
TATAACTGGTATAAGCTTTTGGCTGAGTTTGATGGAGAAAGACATTCCAC
TATATCAACAGTGTTTAGCAGTTATCCAGCAATCATTCCCGATTAGGTGG
GAGGCTGTTTCAGTAAAAGGAGGATACAAGCAGAAGTGGAGTACTAGAGG
TGATGGGCTCCCAAAAGATACCCGAATTTCAGACTCCTTGGCCCCAATCG
GGAACTGGATCAGATCTCTGGAATTGGTCCGAAACCAAGTTCGTCTAAAT
CCATTCAATGAGATCTTGTTCAATCAGCTATGTCGTACAGTGGATAATCA
TTTGAAATGGTCAAATTTGCGAAGAAACACAGGAATGATTGAATGGATCA
ATAGACGAATTTCAAAAGAAGACCGGTCTATACTGATGTTGAAGAGTGAC
CTACACGAGGAAAACTCTTGGAGAGATTAA.

### Example 2. NPL Complex Can Achieve Self-Amplification of reRNA™ in Cells

In vitro delivery based on reRNA™ was performed on 293T cells in the similar manner as described in Example 1. The present example merely differed from Example 1 in that, the reRNA™ RNA sequence carrying at least one of the RNA sequence (base sequence as set forth in SEQ ID NO: 4) encoding the N protein of the Indian strain (IND) of the vesicular stomatitis virus (VSV), the RNA sequence (base sequence as set forth in SEQ ID NO: 5) encoding the P protein of the Indian strain (IND) of the vesicular stomatitis virus (VSV), and the RNA sequence (base sequence as set forth in SEQ ID NO: 6) encoding the L protein of the Indian strain (IND) of the vesicular stomatitis virus (VSV) was constructed. The specific reRNA™ RNA sequences were as follows:

### IND-N-GFP;

### IND-P-GFP;

### IND-L-GFP;

### IND-P+L-GFP;

### IND-N+L-GFP;

### IND-N+P-GFP; and

### IND-N+P+L-GFP.

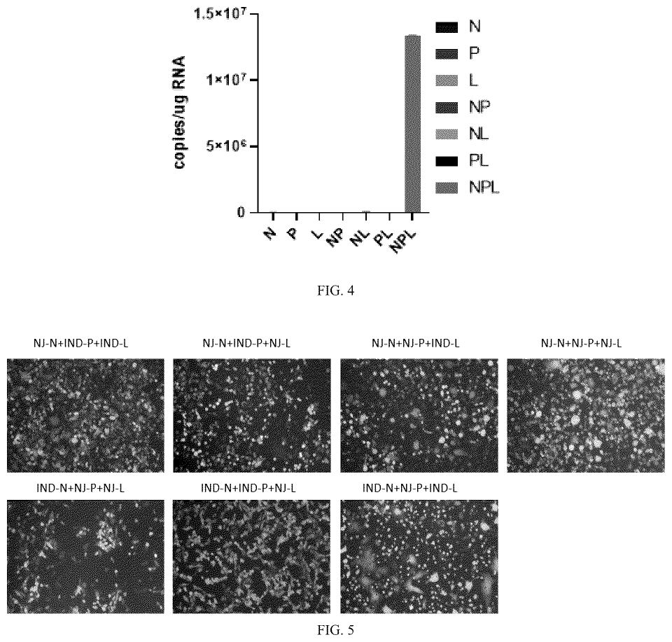

The 293T cells were cultured for 24 hours to observe the fluorescence signal of green fluorescent protein. The results were shown in FIG. 4. As shown in FIG. 4, any individual protein and a combination of two proteins of the N protein, P protein and L protein cannot initiate the self-amplification and expression of the reRNA™-loaded gene (GFP), and the self-amplification and mRNA synthesis of the reRNA™-loaded gene (GFP) can only be initiated in presence of NPL.

### Example 3. NPL Proteins Among Different Strains of Vesicular Stomatitis Virus Can be Freely Combined to Achieve the Expression of reRNA™ Loaded Gene in Cells

According to the information in the GeneBank database, the RNA sequence (base sequence as set forth in SEQ ID NO: 16) encoding N protein of New Jersey strain (NJ) of vesicular stomatitis virus (VSV), the RNA sequence (base sequence as set forth in SEQ ID NO: 17) encoding P protein of New Jersey strain (NJ) of vesicular stomatitis virus (VSV), and the RNA sequence (base sequence as set forth in SEQ ID NO: 18) encoding L protein of New Jersey strain (NJ) of vesicular stomatitis virus (VSV) were further obtained, wherein, the amino acid sequences of N protein, P protein and L protein of the New Jersey strain (NJ) of vesicular stomatitis virus (VSV) obtained by expression of the above coding sequence were as set forth in SEQ ID NO: 13, SEQ ID NO: 14, and SEQ ID NO: 15, respectively. In vitro delivery based on reRNA™ was performed on 293T cells in the similar manner as described in Example 1 In this example, reRNA™ RNA molecules loaded with GFP were prepared, and the coding genes of N, P and L proteins in the core regions were respectively derived from different combinations of the above-mentioned Indian strain (IND) of vesicular stomatitis virus in Example 2 and the above-mentioned New Jersey strain (NJ) of vesicular stomatitis virus. Specifically, the following reRNA™ RNA sequences carrying the following sequences were constructed (the following RNA sequences all contain GFP coding sequence, which is not present for convenience of description):

### NJ-N+IND-P+IND-L;

### NJ-N+IND-P+NJ-L;

### NJ-N+NJ-P+IND-L;

### NJ-N+NJ-P+NJ-L;

### IND-N+NJ-P+NJ-L;

### IND-N+IND-P+NJ-L; and

### IND-N+NJ-P+IND-L.

The same dose of the above-mentioned reRNA™ was introduced into 293T cells, and the cells were cultured for 24 hours after transfection. The expression of loaded gene GFP was confirmed by observing the fluorescence signal, and the self-replication of GFP was confirmed by qPCR.

The experimental results were shown in FIG. 5. The results indicate that different NPL combinations from two vesicular stomatitis viruses can initiate the expression of loaded gene (GFP).

### Example 4. The Combination of NPL Proteins of Three Strains of Vesicular Stomatitis Virus Can Also Achieve the Expression of reRNA™ in Cells

In this example, the Applicant found that any combination of N, P and L proteins of the above three strains of vesicular stomatitis virus can effectively initiate the expression of loaded gene (GFP). Specifically, according to the information in GeneBank database, the coding gene (base sequence as set forth in SEQ ID NO: 23) of P protein of the Cocal strain (COC) of vesicular stomatitis virus (VSV) was further acquired, and the obtained amino acid sequence of P protein of the Cocal strain (COC) of vesicular stomatitis virus (VSV) was as set forth in SEQ ID NO: 20. In vitro delivery based on reRNA™ was performed on 293T cells in substantially the same manner as described in Example 2. In the present example, reRNA™ RNA molecules containing GFP and N protein derived from the New Jersey strain (NJ) of vesicular stomatitis virus described in Example 3, P protein derived from the Cocal strain (COC), and L protein derived from the Indiana strain (IND) described in Examples 1 and 2 were prepared as examples to carry out the experiment.

The above-mentioned reRNA™ was introduced into 293T cells, and the cells were cultured for 24 hours after transfection, and the expression of GFP was confirmed by observing the fluorescence signal.

The experimental results were shown in FIG. 6. The results indicate that the combinations of NPL derived from three strains of vesicular stomatitis virus can effectively initiate the expression of loaded gene (GFP).

### Example 5. NPL Protein of Vesicular Stomatitis Virus Can be Combined with NPL of Other Rhabdovirus (Rabies Virus) to Achieve Intracellular Expression of reRNA™

In vitro delivery based on reRNA™ was performed on 293T cells in substantially the same manner as described in Example 2. In this example, the reRNA™ RNA molecules containing the loaded GFP were prepared, and the NPL genes in the core region were derived from any one of different combinations of vesicular stomatitis virus and rabies virus. The amino acid sequences of N protein, P protein, and L protein of Rabies virus were as set forth in SEQ ID NO: 25, SEQ ID NO: 26, and SEQ ID NO: 27, respectively. The RNA sequences encoding the N protein, P protein and L protein of Rabies virus were as set forth in SEQ ID NO: 28, SEQ ID NO: 29, and SEQ ID NO: 30, respectively.

Different reRNA™ with the same dose were introduced into 293T cells, and the cells were cultured for 24 hours after transfection. The expression of loaded gene GFP was confirmed by observing the fluorescence signal. The results indicate that different combinations of NPL derived from different Rhabdoviruses can initiate the expression of loaded gene (GFP).

### Example 6. Intracellular Expression of reRNA™ Can be Achieved by Combining Proteins with Similar Functions as NPL Proteins

In vitro delivery based on reRNA™ was performed on 293T cells in substantially the same manner as described in Example 2. In this example, the reRNA™ molecules containing loaded GFP were prepared, and the NPL in the core region carried different conservative mutation of RNA sequences encoding N, P, and L proteins, as set forth in SEQ ID NO: 10, SEQ ID NO: 11, and SEQ ID NO: 12 respectively. The amino acid sequences of the obtained N, P, and L proteins were as set forth in SEQ ID NO: 7, SEQ ID NO: 8, and SEQ ID NO: 12, respectively. The 293T cells were transfected with the same dose of different reRNA™ and the cells were cultured for 24 hours after transfection. The expression of loaded gene GFP was confirmed by observing the fluorescence signal. The results indicate that all the different NPL combinations carrying conservative mutations can initiate the expression of GFP.

### Example 7. reRNA™ Can Achieve the Expression of mGM-CSF Secretory Protein

In vitro delivery based on reRNA™ was performed on mouse MC38 cells in substantially the same manner as described in Example 2. The present example differed from Example 2 merely in that: reRNA™ containing loaded mGM-CSF (mouse granulocyte-macrophage colony stimulating factor) was prepared, and the core region was NPL (derived from Indian strain (IND) of vesicular stomatitis virus described in Examples 1 and 2). The reRNA™ loaded with mGM-CSF was introduced into mouse MC38 cells, and the cell culture supernatant was collected at different times to detect mGM-CSF secretion. The results indicate that it can effectively initiate the expression of mGM-CSF and make it secrete to the outside of the cells.

### Example 8. reRNA™ Can Achieve the Presentation of OVA Antigen Peptide in Antigen-Presenting Cells (APCs)

The reRNA™ (reRNA™ OVA group) and reRNA™ (Blank group) containing loaded RNA sequences encoding OVA antigen were prepared. The core region of reRNA™ was NPL (derived from Indian strain (IND) of vesicular stomatitis virus described in Example 1 and Example 2), and the amino acid sequence of OVA antigen obtained by expression was as set forth in SEQ ID NO: 42. Specifically, reRNA™ was introduced into C57 mouse spleen cells, and the mouse spleen cells were recovered after 24 hours of culture. The antigen-presenting cells were tested for OVA antigen presentation by flow cytometry.

(SEQ ID NO: 42)
MGSIGAASMEFCFDVFKELKVHHANENIFYCPIAIMSALAMVYLGAKDST
RTQINKVVRFDKLPGFGDSIEAQCGTSVNVHSSLRDILNQITKPNDVYSF
SLASRLYAEERYPILPEYLQCVKELYRGGLEPINFQTAADQARELINSWV
ESQINGIIRNVLQPSSVDSQTAMVLVNAIVFKGLWEKAFRDEDTQAMPFR
VTERESKPVQMMYQTGLFRVASMASEKMKILELPFASGTMSMLVLLPDEV
SGLEQLESIINFEKLTEWTSSNVMEERKIKVYLPRMKMEEKYNLTSVLMA
MGITDVFSSSANLSGISSAESLKISQAVHAAHAEINEAGREVVGSAEAGV
DAASVSEEFRADHPFLFCIKHIATNAVLFFGRCVSP

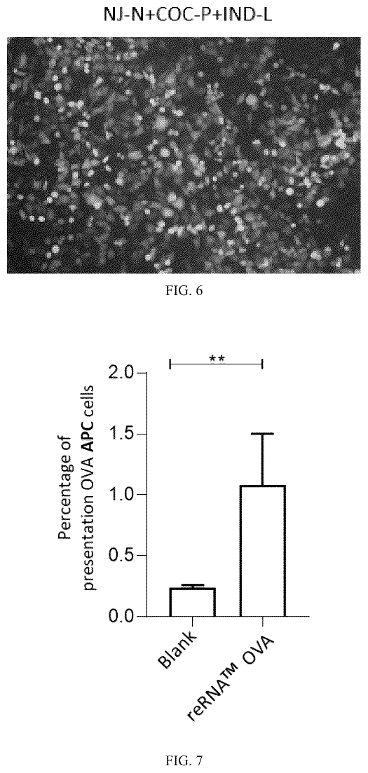

The experimental results were shown in FIG. 7. The results indicate that antigen-presenting cells can present OVA antigen. The results also indicate that the OVA antigen carried by reRNA™ can not only be expressed in APC cells, but also be processed and presented to the cell surface by APC cells.

In the specification, descriptions referring to the terms “an embodiment”, “some embodiments”, “examples”, “specific examples” or “some examples” mean that specific features, structures, materials or characteristics described in connection with this embodiment or example are included in at least one embodiment or example of the present disclosure. In this specification, the schematic expressions of the above terms are not necessarily directed to the same embodiment or example. Moreover, the specific features, structures, materials or characteristics described may be combined in a suitable manner in any one or more embodiments or examples. In addition, those skilled in the art can combine and integrate different embodiments or examples and features of different embodiments or examples described in this specification without contradicting each other.

Although the embodiments of the present disclosure have been illustrated and described above, it can be understood that the above embodiments are exemplary and should not be understood as limitations of the present disclosure. Those skilled in the art can make changes, modifications, substitutions and variations to the above embodiments within the scope of the present disclosure.

What is claimed is:

1. A self-replicating RNA molecule, comprising:
a first RNA sequence, encoding an N protein or a functional fragment of the N protein,
a second RNA sequence, encoding a P protein or a functional fragment of the P protein, and
a third RNA sequence, encoding an L protein or a functional fragment of the L protein,
wherein the self-replicating RNA molecule is suitable for producing the N protein, the P protein, the L protein, or the functional fragments thereof in animal cells.

2. The self-replicating RNA molecule according to claim 1, wherein at least one of the N protein, the P protein, and the L protein is independently derived from Rhabdoviridae virus.

3. The self-replicating RNA molecule according to claim 2, wherein the Rhabdoviridae virus comprises at least one virus selected from Vesiculorius, Lyssavirus, and Ephemerovirus.

4. The self-replicating RNA molecule according to claim 2, wherein the Rhabdoviridae virus comprises at least one virus selected from Vesicular stomatitis New Jersey virus, Maraba virus, Vesicular stomatitis Alagoas virus Indiana 3, Cocal virus Indiana 2, Isfahan virus, Chandipura virus, Spring viraemia of carp virus, American bat vesiculovirus, Carajas vesiculovirus, Jurona vesiculovirus, Malpais Spring vesiculovirus, Morreton vesiculovirus, Perinet vesiculovirus, Piry vesiculovirus, Radi vesiculovirus, Yug Bogdanovac vesiculovirus, West Caucasian bat virus. Aravan lyssavirus, Khujand lyssavirus, Rabies virus, Australian bat lyssavirus, European bat lyssavirus 1, European bat lyssavirus 2, Irkut virus, Shimoni bat virus, Lagos bat virus, Mokola virus, Lleida bat lyssavirus, Ikoma lyssavirus, Gannoruwa bat lyssavirus, Bokeloh bat lyssavirus, Duvenhage lyssavirus, Bovine fever ephemerovirus, Adelaide River ephemerovirus, Berrimah ephemerovirus, and Kimberley ephemerovirus.

5. The self-replicating RNA molecule according to claim 1, wherein:
the N protein has an amino acid sequence as set forth in SEQ ID NO: 1, SEQ ID NO: 7, SEQ ID NO: 13, SEQ ID NO: 19, or SEQ ID NO: 25, or an amino acid sequence having at least 75%, at least 80%, at least 85%, at least 90%, at least 95%, or at least 99% identity with the amino acid sequence as set forth in SEQ ID NO: 1, SEQ ID NO: 7, SEQ ID NO: 13, SEQ ID NO: 19, or SEQ ID NO: 25;
the P protein has an amino acid sequence as set forth in SEQ ID NO: 2, SEQ ID NO: 8, SEQ ID NO: 14, SEQ ID NO: 20, or SEQ ID NO: 26, or an amino acid sequence having at least 75%, at least 80%, at least 85%, at least 90%, at least 95%, or at least 99% identity with the amino acid sequence as set forth in SEQ ID NO: 2, SEQ ID NO: 8, SEQ ID NO: 14, SEQ ID NO: 20, or SEQ ID NO: 26; and
the L protein has an amino acid sequence as set forth in SEQ ID NO: 3, SEQ ID NO: 9, SEQ ID NO: 15, SEQ ID NO: 21, or SEQ ID NO: 27, or an amino acid sequence having at least 75%, at least 80%, at least 85%, at least 90%, at least 95%, or at least 99% identity with the amino acid sequence as set forth in SEQ ID NO: 3, SEQ ID NO: 9, SEQ ID NO: 15, SEQ ID NO: 21, or SEQ ID NO: 27.

6. The self-replicating RNA molecule according to claim 5, wherein:
the N protein has the amino acid sequence as set forth in SEQ ID NO: 1, the P protein has the amino acid sequence as set forth in SEQ ID NO: 2, and the L protein has the amino acid sequence as set forth in SEQ ID NO: 3; or
the N protein has the amino acid sequence as set forth in SEQ ID NO: 7, the P protein has the amino acid sequence as set forth in SEQ ID NO: 8, and the L protein has the amino acid sequence as set forth in SEQ ID NO: 9; or
the N protein has the amino acid sequence as set forth in SEQ ID NO: 13, the P protein has the amino acid sequence as set forth in SEQ ID NO: 14, and the L protein has the amino acid sequence as set forth in SEQ ID NO: 15; or
the N protein has the amino acid sequence as set forth in SEQ ID NO: 19, the P protein has the amino acid sequence as set forth in SEQ ID NO: 20, and the L protein has the amino acid sequence as set forth in SEQ ID NO: 21; or
the N protein has the amino acid sequence as set forth in SEQ ID NO: 25, the P protein has the amino acid sequence as set forth in SEQ ID NO: 26, and the L protein has the amino acid sequence as set forth in SEQ ID NO: 27.

7. The self-replicating RNA molecule according to claim 1, wherein:
the first RNA sequence has a base sequence as set forth in SEQ ID NO: 4, SEQ ID NO: 10, SEQ ID NO: 16, SEQ ID NO: 22, or SEQ ID NO: 28, or has a base sequence having at least 75%, at least 80%, at least 85%, at least 90%, at least 95%, or at least 99% identity with the base sequence as set forth in SEQ ID NO: 4, SEQ ID NO: 10, SEQ ID NO: 16, SEQ ID NO: 22, or SEQ ID NO: 28;
the second RNA sequence has a base sequence as set forth in SEQ ID NO: 5, SEQ ID NO: 11, SEQ ID NO: 17, SEQ ID NO: 23, or SEQ ID NO: 29, or has a base sequence having at least 75%, at least 80%, at least 85%, at least 90%, at least 95%, or at least 99% identity with the base sequence as set forth in SEQ ID NO: 5, SEQ ID NO: 11, SEQ ID NO: 17, SEQ ID NO: 23, or SEQ ID NO: 29; and
the third RNA sequence has a base sequence as set forth in SEQ ID NO: 6, SEQ ID NO: 12, SEQ ID NO: 18, SEQ ID NO: 24, or SEQ ID NO: 30, or has a base sequence having at least 75%, at least 80%, at least 85%, at least 90%, at least 95%, or at least 99% identity with the base sequence as set forth in SEQ ID NO: 6, SEQ ID NO: 12, SEQ ID NO: 18, SEQ ID NO: 24, or SEQ ID NO: 30.

8. The self-replicating RNA molecule according to claim 7, wherein:
the first RNA sequence has the base sequence as set forth in SEQ ID NO: 4, the second RNA sequence has the base sequence as set forth in SEQ ID NO: 5, and the third RNA sequence has the base sequence as set forth in SEQ ID NO: 6; or
the first RNA sequence has the base sequence as set forth in SEQ ID NO: 10, the second RNA sequence has the base sequence as set forth in SEQ ID NO: 11, and the third RNA sequence has the base sequence as set forth in SEQ ID NO: 12; or
the first RNA sequence has the base sequence as set forth in SEQ ID NO: 16, the second RNA sequence has the base sequence as set forth in SEQ ID NO: 17, and the third RNA sequence has the base sequence as set forth in SEQ ID NO: 18; or
the first RNA sequence has the base sequence as set forth in SEQ ID NO: 22, the second RNA sequence has the base sequence as set forth in SEQ ID NO: 23, and the third RNA sequence has the base sequence as set forth in SEQ ID NO: 24; or
the first RNA sequence has the base sequence as set forth in SEQ ID NO: 28, the second RNA sequence has the base sequence as set forth in SEQ ID NO: 29, and the third RNA sequence has the base sequence as set forth in SEQ ID NO: 30.

9. The self-replicating RNA molecule according to claim 1, further comprising:
a target molecule coding region, encoding at least one target molecule.

10. The self-replicating RNA molecule according to claim 9, wherein the target molecule is suitable to serve as at least one of nucleic acid medicaments, protein medicaments, pathogen vaccines, tumor vaccines, and therapeutic agents for rare diseases.

11. The self-replicating RNA molecule according to claim 9, wherein:
the target molecule coding region encodes at least one of fluorescent protein, granulocyte-macrophage colony stimulating factor, and OVA; and
optionally, the fluorescent protein comprises at least one of green fluorescent protein, red fluorescent protein, and yellow fluorescent protein.

12. A DNA molecule, encoding the self-replicating RNA molecule according to claim 1.

13. An expression vector, carrying the DNA molecule according to claim 12.

14. A method for preparing the self-replicating RNA molecule according to claim 1, the method comprising:
expressing, using a DNA molecule encoding the self-replicating RNA molecule or an expression vector carrying a DNA molecule encoding the self-replicating RNA molecule, the self-replicating RNA molecule in host cells; and
collecting the self-replicating RNA molecule.

15. An RNA-protein complex, comprising:
the self-replicating RNA molecule according to claim 1; and
a protein, comprising the N protein or functional fragment thereof, the P protein or functional fragment thereof, and the L protein or functional fragment thereof.

16. A pharmaceutical composition, comprising
the self-replicating RNA molecule according to claim 1; or
an DNA molecule encoding the self-replicating RNA molecule; or
an expression vector carrying an DNA molecule encoding the self-replicating RNA molecule; or
an RNA-protein complex, comprising the self-replicating RNA molecule and a protein, wherein the protein comprises the N protein or functional fragment thereof, the P protein or functional fragment thereof, and the L protein or functional fragment thereof.

17. A method for expressing a target molecule in an individual, the method comprising:
administering to the individual the self-replicating RNA molecule according to claim 1.
